(19) United States
(12) Patent Application Publication
(10) Pub. No.: US 2025/0320253 A1
(43) Pub. Date: Oct. 16, 2025

(54) TRANSMEMBRANE PEPTIDIC ANTAGONISTS OF PLEXIN-A1 AND THEIR THERAPEUTIC USES

(71) Applicants: UNIVERSITE DE STRASBOURG, Strasbourg (FR); INSTITUT NATIONAL DE LA SANTE ET DE LA RECHERCHE MEDICALE (INSERM), Paris (FR)

(72) Inventors: Dominique BAGNARD, Ammerschwihr (FR); Fabien BINAME, Geispolsheim (FR)

(21) Appl. No.: 18/690,695

(22) PCT Filed: Sep. 7, 2022
(86) PCT No.: PCT/EP2022/074907

§ 371 Date: Mar. 8, 2024

(30) Foreign Application Priority Data
Sep. 8, 2021 (EP) 21306229.2

(51) Int. Cl. C07K 14/00; A61K 31/137; A61K 38/00; A61P 25/28

(52) U.S. Cl. CPC C07K 14/001; A61K 31/137; A61P 25/28; A61K 38/00

(57) ABSTRACT

The present application relates to peptides derived from the transmembrane domain of Plexin-A1 that inhibits Neu-ropilin-1/Plexin-A1 heterodimerization. These peptides neutralize the inhibitory effect of Sema3A on cell migration and angiogenesis, and may be useful for the treatment of diseases associated with Sema3A and/or Neuropilin-1/Plexin-A1 activity, such as demyelinating diseases and diseases associated with abnormal angiogenesis such as cancer.

### CROSS REFERENCE TO RELATED APPLICATIONS

The present application claims the benefits of EP patent application Ser. No. 21/306,229 filed on Sep. 8, 2021, which is incorporated herein by reference.

### FIELD OF THE INVENTION

The present disclosure relates to the treatment of diseases and conditions relating to the activity of the Neuropilin/Plexin-A1 receptor, such as neurodegenerative diseases.

### BACKGROUND OF THE INVENTION

Multiple sclerosis (MS) is the most common chronic neurological disorder in young adults and affects nearly one million people in the United States. MS is an autoimmune disease in which the patient's own immune cells attack and destroy the myelin sheath that protects neurons in the brain. The remyelination of these neurons by oligodendrocytes is a spontaneous phenomenon that ceases to be effective with the advancement of the disease, subsequently causing irreparable nerve damage and progressive disability. Compounds currently approved for the treatment of MS are designed to limit destructive immune attack.

Multiple sclerosis experts agree that drugs that stimulate the regeneration of myelin sheaths produced by oligodendrocytes represent an innovative approach that benefits MS patients by protecting neurons to prevent further damage and eventually restore neuronal function.

Among the many factors regulating the initial phases of myelination, members of the semaphorin family have been shown to regulate the migration of oligodendrocytes precursor cells and inhibit their maturation. Among them, although the role of sema3A as an inhibitory regulator of myelination is recognized, the importance of the receptors involved is not yet clear.

WO2007/000672 describes transmembrane peptide with antagonistic activity of the Semaphorins/neuropilins complex. Plexin-A1 is one of the origins of the peptides, among others, with no specific therapeutic interest associated with it.

Biname et al (EMBO Mol Med (2019) 11: e10378) showed that Plexin-A1, the signaling receptor of the oligodendrocyte inhibitor Semaphorin 3A, is overexpressed in MS patients. The authors describe a peptidic antagonist antagonizing Plexin-A1 (called MTP-PlexA1) that is able to counteract the Sema3A inhibitory effect on oligodendrocyte migration and differentiation in vitro. MTP-PlexA1 is a synthetic peptide mimicking the transmembrane domain of Plexin-A1 (TLPAIVGIGGGGGLLLLVIVAVLIAYKRK, SEQ ID NO: 1). The administration of the peptide also showed protective effects, leading to a reduced severity of demyelination in the context of experimental autoimmune encephalitis (EAE).

However, there remains a need of identification of new antagonistic peptides.

### SUMMARY OF THE INVENTION

The present disclosure provides new peptides blocking the Plexin-A1 receptor involved in the inhibitory signaling pathway Sema3A-neuropilin 1-Plexin-A1. The peptides inhibit the interaction between Neuropilin-1 and Plexin-A1 and the inhibitory effect of Sema3A on cell migration. These peptides are capable of increasing remyelination by inhibiting one of the pathways involved in blocking the different stages of remyelination and have a protective effect on experimental animal models of multiple sclerosis (EAE-PLP and EAE-MOG) models, thereby demonstrating their therapeutic potential for the treatment of demyelinating diseases such as multiple sclerosis. In addition, the inventors provide evidence of the capacity of the peptides to inhibit angiogenesis, thereby expanding the potential use of blocking peptides in other diseases involving abnormal vascularization such as tumor growth and metastasis, hemangiomas, psoriasis, Kaposi's sarcoma, ocular neovascularization, Rheumatoid arthritis, endometriosis, or atherosclerosis.

The inventors identified the rules for designing an antagonistic peptide. In the peptides, the motif GxxxG is involved in the activity. It was found that the first glycine can be replaced by a serine. In addition, another important aspect for the design of the peptides is the anchorage of the motif G/SxxxG at the membrane and the distance between the membrane surface and the motif G/SxxxG. Indeed, the position of the motif G/SxxxG is key. More specifically, if the peptide has a N-terminal membrane anchoring, five amino acids should be inserted between the N-terminal membrane anchoring motif and the motif G/SxxxG. If the peptide has a C-terminal membrane anchoring, then thirteen amino acids should be inserted between the motif G/SxxxG and the C-terminal membrane anchoring motif with positively charged amino acids and eleven amino acids should preferably be inserted between the motif G/SxxxG and the C-terminal membrane anchoring motif with negatively charged amino acids. Once these rules have been determined, the inventors surprisingly observed that the peptides can be shortened up to the motif G/SxxxG at the opposite side of the membrane anchoring motif. This aspect allows the design of shorter peptides.

The present disclosure relates to a peptide, preferably a peptide of 50, 45 or 40 amino acids or less, comprising, consisting essentially of or consisting of:
a first domain of the sequence G/S-X-X-X-G,
a second domain of the sequence: -(X)₁₁-(CterpolyD/E) directly linked at C-terminal end of the motif (i.e., G/S-X-X-X-G-(X)₁₁-(CterpolyD/E); (NterGp)-(X)₅- directly linked at N-terminal end of the motif (i.e., (NterGp)-(X)₅-G/S-X-X-X-G); or -(X)₁₃-(CterGp) directly linked at C-terminal end of the motif (i.e., G/S-X-X-X-G-(X)₁₃-(CterGp); and
with X being any amino acid (D- or L-) but no more than 2 among the X being a charged amino acid;
   with CterpolyD/E being a group of 4-10 amino acids (D- or L-) including at least 2 negatively charged amino acids; NterGp being a group of 3-5 amino acids (D- or L-) including at least 2 charged amino acids, and CterGp being a group of 3-5 amino acids (D- or L-) including at least 3 positively charged amino acids;
   or a retro or retroinverso sequence thereof,
   or a pharmaceutically acceptable salt thereof,
   wherein the peptide does not comprise or consist of the sequence TLPAIVGIGGGGGLLLLVIVAVLIAYKRK (SEQ ID NO: 1).

In an embodiment, the peptide has a length of 30 amino acids or less. In an embodiment, the peptide has a length of 28 amino acids or less. In an embodiment, the peptide has a length of 26 amino acids or less. In an embodiment, the peptide has a length of 25 amino acids or less. In an embodiment, the peptide has a length of 20 amino acids or less.

The present disclosure relates to a peptide comprising, consisting essentially of or consisting of:
a motif G/S-X-X-X-G,
-(X)₁₁-(CterpolyD/E) directly linked at C-terminal end of the motif (i.e., G/S-X-X-X-G-(X)₁₁-(CterpolyD/E); (NterGp)-(X)₅-directly linked at N-terminal end of the motif (i.e., (NterGp)-(X)₅-G/S-X-X-X-G); or -(X)₁₃-(CterGp) directly linked at C-terminal end of the motif (i.e., G/S-X-X-X-G-(X)₁₃-(CterGp); and
with X being any amino acid (D- or L-) but no more than 2 among the X being a charged amino acid;
with NterGp being a group of 3-5 amino acids (D- or L-) including at least 2 charged amino acids, CterGp being a group of 3-5 amino acids (D- or L-) including at least 3 positively charged amino acids, and CterpolyD/E being a group of 4-10 amino acids (D- or L-) including at least 2 negatively charged amino acids,
   or a retro or retroinverso analog thereof,
   or a pharmaceutically acceptable salt thereof,
   wherein the peptide inhibits the inhibitory effect of Sema3A on migration and/or inhibits the interaction between Neuropilin-1 and Plexin-A1 as measured by the method detailed in the specification; and
   wherein the peptide has not the sequence of MTP-PlexA1 (TLPAIVGIGGGGGLLLLVIVAVLIAYKRK, SEQ ID NO: 1).

More specifically, the inventors identified a new class of antagonistic peptides with advantageous properties in the context of transmembrane peptides. These peptides present an improved solubility and stability relative MTP-PlexA1. They present a plasmatic half-life longer than 24 h and a biodistribution suitable for reaching target organs such as brain and spinal cord.

In this advantageous aspect, the peptide comprises, consists essentially of or consists of a sequence selected from:

(SEQ ID NO: 2)
G/S-I/G/L-G/V-G-G-G/V-G/V-L/E-L-L-L/E-V-I-
V-A/E-V-(CterpolyD/E)
(SEQ ID NO: 3)
V/T-G/S-I/G/L-G/V-G-G-G/V-G/V-L/E-L-L-L/E-
V-I-V-A/E-V-(CterpolyD/E)
(SEQ ID NO: 4)
I/L-V/T-G/S-I/G/L-G/V-G-G-G/V-G/V-L/E-L-L-
L/E-V-I-V-A/E-V-(CterpolyD/E)
(SEQ ID NO: 5)
A/V-I/L-V/T-G/S-I/G/L-G/V-G-G-G/V-G/V-L/E-
L-L-L/E-V-I-V-A/E-V-(CterpolyD/E)
(SEQ ID NO: 6)
P/I-A/V-I/L-V/T-G/S-I/G/L-G/V-G-G-G/V-G/V-
L/E-L-L-L/E-V-I-V-A/E-V-(CterpolyD/E)
(SEQ ID NO: 7)
I/L-P/I-A/V-I/L-V/T-G/S-I/G/L-G/V-G-G-G/V-
G/V-L/E-L-L-L/E-V-I-V-A/E-V-(CterpolyD/E)
and
(SEQ ID NO: 8)
T-I/L-P/I-A/V-I/L-V/T-G/S-I/G/L-G/V-G-G-
G/V-G/V-L/E-L-L-L/E-V-I-V-A/E-V-(CterpolyD/E);

wherein CterpolyD/E is a group of 4-10 amino acids including at least 2 negatively charged amino acids, and
wherein the sequence may comprise 1, 2 or 3 substitutions of one amino acid at any position except the bold residues and the addition of 1 to 6 amino acids at the N-terminal or C-terminal end.

Optionally, CterpolyD/E has a sequence of -X″1-X″2-(Z)ₙ with X″1 being a small amino acid (e.g., G or A), X″2 being an aromatic amino acid (e.g., Y or W), Z being a negatively charged residue (e.g., D or E), and n being an integer between 2-10, preferably 4-6; or a sequence of -X″1-X″1bis-X″2-(E)ₙ with X″1 and X″1bis being long aliphatic amino acids such as I or L, X″2 being an aromatic amino acid (e.g., Y or W), and n being an integer between 2-10, preferably 4-6.

Optionally, the peptide comprises, consists essentially of or consists of the amino acid sequence:

(SEQ ID NO: 9)
G/S-I/G/L-G/V-G-G-G/V-G/V-L/E-L-L-
L/E-V-I-V-E-V-A/LI-Y-(D/E)ₙ

wherein the sequence may further comprise 1, 2 or 3 substitutions of one amino acid at any position except the bold residues and the addition of 1 to 6 amino acids at the N-terminal and/or C-terminal end, and wherein n is an integer between 2-10, preferably 4-6.

Optionally, the peptide comprises, consists essentially of or consists of a sequence selected from:

(SEQ ID NO: 10)
AIT-G/S-LVGGVGLLLEVIVEVAYEEEEE
(SEQ ID NO: 11)
T-G/S-LVGGVGLLLEVIVEVAYEEEEE;
(SEQ ID NO: 12)
G/S-LVGGVGLLLEVIVEVAYEEEEE;
(SEQ ID NO: 13)
TLPAIV-G/S-IGGGGGELLLVIVEVLIYEEEEE;
(SEQ ID NO: 14)
TLPAIT-G/S-LVGGVGLLLEVIVEVAYEEEEE;
and
(SEQ ID NO: 15)
TLPAIV-G/S-IGGGVVLLLEVIVEVAYEEEEE;

wherein the sequence may further comprise 1, 2 or 3 substitutions of one amino acid at any position except the bold residues and the addition of 1 to 6 amino acids at the N-terminal and/or C-terminal end.

More specifically, the peptide comprises, consists essentially of or consists of a sequence selected from:

(SEQ ID NO: 16)
AITGLVGGVGLLLEVIVEVAYEEEEE;
(SEQ ID NO: 17)
TGLVGGVGLLLEVIVEVAYEEEEE;
(SEQ ID NO: 18)
GLVGGVGLLLEVIVEVAYEEEEE;
(SEQ ID NO: 19)
TLPAIVGIGGGGGELLLVIVEVLIYEEEEE;
(SEQ ID NO: 20)
TLPAITGLVGGVGLLLEVIVEVAYEEEEE;
(SEQ ID NO: 21)
TLPAIVSIGGGVVLLLEVIVEVAYEEEEE;
(SEQ ID NO: 98)
TLPAITGLVGGVGLLLEVIVEVAYEE;
(SEQ ID NO: 100)
TLPAITGLVGGVVLLLEVIVEVAYEEEEE;
(SEQ ID NO: 101)
TLPAITGLVGGVGLVLEVIVEVAYEEEEE;
(SEQ ID NO: 106)
_{d}E_{d}E_{d}E_{d}E_{d}E_{d}Y_{d}A_{d}V_{d}E_{d}V_{d}l_{d}V_{d}E_{d}L_{d}L_{d}LG_{d}VGG_{d}V_{d}LG_{d}T_{d}I_{d}A_{d}P_{d}L_{d}T;
(SEQ ID NO: 107)
TLPAITGLVGGVGLLLEVIVEVAYDD;
(SEQ ID NO: 108)
TLPAITGLVGGVGLLLEVIVEVAYDEDED;
(SEQ ID NO: 111)
_{d}TLPAITGLVGGVGLLLEVIVEVAYEEEEE;
(SEQ ID NO: 112)
TLPAITGLVGGVGLLLEVIVEVAYEEEEE;
or
(SEQ ID NO: 113)
TLPAITGLVGGVGLLVEVIVEVAYEEEEE;

wherein the sequence may further comprise 1, 2 or 3 substitutions of one amino acid at any position except the bold residues and the addition of 1 to 6 amino acids at the N-terminal or C-terminal end.

In an embodiment, the peptide comprises, consists essentially of or consists of a sequence selected from:

(SEQ ID NO: 16)
AITGLVGGVGLLLEVIVEVAYEEEEE;
(SEQ ID NO: 17)
TGLVGGVGLLLEVIVEVAYEEEEE;
(SEQ ID NO: 18)
GLVGGVGLLLEVIVEVAYEEEEE;
(SEQ ID NO: 19)
TLPAIVGIGGGGGELLLVIVEVLIYEEEEE;
(SEQ ID NO: 20)
TLPAITGLVGGVGLLLEVIVEVAYEEEEE;
(SEQ ID NO: 21)
TLPAIVSIGGGVVLLLEVIVEVAYEEEEE;
(SEQ ID NO: 97)
TLPAITGLVGGVGLLLEVIVEVAYEEE;
(SEQ ID NO: 98)
TLPAITGLVGGVGLLLEVIVEVAYEE;
(SEQ ID NO: 100)
TLPAITGLVGGVVLLLEVIVEVAYEEEEE;
(SEQ ID NO: 101)
TLPAITGLVGGVGLVLEVIVEVAYEEEEE;
(SEQ ID NO: 106)
_{d}E_{d}E_{d}E_{d}E_{d}E_{d}Y_{d}A_{d}V_{d}E_{d}V_{d}l_{d}V_{d}E_{d}L_{d}L_{d}LG_{d}VGG_{d}V_{d}LG_{d}T_{d}I_{d}A_{d}P_{d}L_{d}T;
(SEQ ID NO: 107)
TLPAITGLVGGVGLLLEVIVEVAYDD;
(SEQ ID NO: 108)
TLPAITGLVGGVGLLLEVIVEVAYDEDED;
(SEQ ID NO: 111)
_{d}TLPAITGLVGGVGLLLEVIVEVAYEEEEE;
(SEQ ID NO: 112)
TLPAITGLVGGVGLLLEVIVEVAYEEEEE;
or
(SEQ ID NO: 113)
TLPAITGLVGGVGLLVEVIVEVAYEEEEE;

wherein the sequence may further comprise 1, 2 or 3 substitutions of one amino acid at any position except the bold residues and the addition of 1 to 6 amino acids at the N-terminal or C-terminal end.

In an embodiment, the peptide comprises, consists essentially of or consists of a sequence selected from:

(SEQ ID NO: 16)
AITGLVGGVGLLLEVIVEVAYEEEEE;
(SEQ ID NO: 17)
TGLVGGVGLLLEVIVEVAYEEEEE;
(SEQ ID NO: 18)
GLVGGVGLLLEVIVEVAYEEEEE;
(SEQ ID NO: 19)
TLPAIVGIGGGGGELLLVIVEVLIYEEEEE;
(SEQ ID NO: 20)
TLPAITGLVGGVGLLLEVIVEVAYEEEEE;
(SEQ ID NO: 21)
TLPAIVSIGGGVVLLLEVIVEVAYEEEEE;
(SEQ ID NO: 100)
TLPAITGLVGGVVLLLEVIVEVAYEEEEE;
(SEQ ID NO: 108)
TLPAITGLVGGVGLLLEVIVEVAYDEDED;
or
(SEQ ID NO: 113)
TLPAITGLVGGVGLLVEVIVEVAYEEEEE;

wherein the sequence may further comprise 1, 2 or 3 substitutions of one amino acid at any position except the bold residues and the addition of 1 to 6 amino acids at the N-terminal or C-terminal end.

In another aspect in which the peptides present a N-terminal membrane anchoring motif (i.e., NterGp), the peptide comprises, consists essentially of or consists of a sequence selected from:

(SEQ ID NO: 22)
(NterGp)-L/I-P/I-A/V-I/L-V/T-G/S-I/G/L-G/V-G-G;
(SEQ ID NO: 23)
(NterGp)-L/I-P/I-A/V-I/L-V/T-G/S-I/G/L-G/V-G-G-
G/V;
(SEQ ID NO: 24)
(NterGp)-L/I-P/I-A/V-I/L-V/T-G/S-I/G/L-G/V-G-G-
G/V-G/V;
(SEQ ID NO: 25)
(NterGp)-L/I-P/I-A/V-I/L-V/T-G/S-I/G/L-G/V-G-G
G/V-G/V-L/E;
(SEQ ID NO: 26)
(NterGp)-L/I-P/I-A/V-I/L-V/T-G/S-I/G/L-G/V-G-G-
G/V-G/V-L/E-L;
(SEQ ID NO: 27)
(NterGp)-L/I-P/I-A/V-I/L-V/T-G/S-I/G/L-G/V-G-G-
G/V-G/V-L/E-L-L;
(SEQ ID NO: 28)
(NterGp)-L/I-P/I-A/V-I/L-V/T-G/S-I/G/L-G/V-G-G-
G/V-G/V-L/E-L-L-L/E;
(SEQ ID NO: 29)
(NterGp)-L/I-P/I-A/V-I/L-V/T-G/S-I/G/L-G/V-G-G-
G/V-G/V-L/E-L-L-L/E-V;
(SEQ ID NO: 30)
(NterGp)-L/I-P/I-A/V-I/L-V/T-G/S-I/G/L-G/V-G-G-
G/V-G/V-L/E-L-L-L/E-V-I;
(SEQ ID NO: 31)
(NterGp)-L/I-P/I-A/V-I/L-V/T-G/S-I/G/L-G/V-G-G-
G/V-G/V-L/E-L-L-L/E-V-I-V;
(SEQ ID NO: 32)
(NterGp)-L/I-P/I-A/V-I/L-V/T-G/S-I/G/L-G/V-G-G-
G/V-G/V-L/E-L-L-L/E-V-I-V-A/E;
(SEQ ID NO: 33)
(NterGp)-L/I-P/I-A/V-I/L-V/T-G/S-I/G/L-G/V-G-G-
G/V-G/V-L/E-L-L-L/E-V-I-V-A/E-V;
(SEQ ID NO: 34)
(NterGp)-L/I-P/I-A/V-I/L-V/T-G/S-I/G/L-G/V-G-G-
G/V-G/V-L/E-L-L-L/E-V-I-V-A/E-V-L;
and
(SEQ ID NO: 35)
(NterGp)-L/I-P/I-A/V-I/L-V/T-G/S-I/G/L-G/V-G-G-
G/V-G/V-L/E-L-L-L/E-V-I-V-A/E-V-L-I;

wherein NterGp is a group of 3-5 amino acids including at least 2 charged amino acids, and wherein the sequence may comprise 1, 2 or 3 substitutions of one amino acid at any position except the bold residues and the addition of 1 to 6 amino acids at the N-terminal or C-terminal end.

Optionally, NterGp has a sequence of 4 amino acids X1-X2-X3-X4 with X1 and X3 being two charged amino acids, optionally one positively charged and the other negatively charged, X2 being a small amino acid (e.g., G or A) and X4 being an aromatic amino acid (e.g., Y or W), more preferably NterGp being KGDW (SEQ ID NO:116).

In another aspect in which the peptides comprise a C-terminal membrane anchoring motif with positively charged amino acids (i.e., CterGp), the peptide comprises, consists essentially of or consists of a sequence selected from:

(SEQ ID NO: 36)
A/V-I/L-V/T-G/S-I/G/L-G/V-G-G-G/V-G/V-L/E-
L-L-L/E-V-I-V-A/E-V-L-I-(CterGp);
(SEQ ID NO: 37)
I/L-V/T-G/S-I/G/L-G/V-G-G-G/V-G/V-L/E-L-L-
L/E-V-I-V-A/E-V-L-I-(CterGp);
(SEQ ID NO: 38)
V/T-G/S-I/G/L-G/V-G-G-G/V-G/V-L/E-L-L-L/E-
V-I-V-A/E-V-L-I-(CterGp);
and
(SEQ ID NO: 39)
G/S-I/G/L-G/V-G-G-G/V-G/V-L/E-L-L-L/E-V-I-
V-A/E-V-L-I-(CterGp);

wherein CterGp is a group of 3-5 amino acids including at least 3 positively charged amino acids,
wherein the sequence may comprise 1, 2 or 3 substitutions of one amino acid at any position except the bold residue.

Optionally, CterGp has a sequence of 5 amino acids X′1-X′2-X′3-X′4-X′5 with X′1 being a small amino acid (e.g., G or A), X′2 being an aromatic amino acid (e.g., Y or W), X′3, X′4 and X′5 being a basic amino acid, more preferably CterGp being a sequence selected from AYKRK (SEQ ID NO: 76), AYKKR (SEQ ID NO: 77), AYKRR (SEQ ID NO: 78), AYRRK (SEQ ID NO: 79) and AYRKK (SEQ ID NO: 80).

Optionally, the peptide comprises, consists essentially of, or consists of an amino acid sequence selected from:

(SEQ ID NO: 40)
KGDWLPAIT-G/S-LVGGVGLL
(SEQ ID NO: 41)
KGDWLPAIV-G/S-IGGGVVLL
(SEQ ID NO: 42)
KGDWIPALV-G/S-GGGGGGLL
(SEQ ID NO: 43)
KGDWLPALV-G/S-IGGGVGLL
(SEQ ID NO: 44)
KGDWIPALV-G/S-LGGGGGLL
(SEQ ID NO: 45)
KGDWLIAIV-G/S-IGGG
(SEQ ID NO: 46)
KGDWLPVIV-G/S-IGGG
(SEQ ID NO: 47)
KGDWLPAIV-G/S-IGGGGGLL
(SEQ ID NO: 48)
KGDWLPAIV-G/S-IGGGGGL
(SEQ ID NO: 49)
KGDWLPAIV-G/S-IGGGGG
(SEQ ID NO: 50)
KGDWLPAIV-G/S-IGGGG
(SEQ ID NO: 51)
KGDWLPAIV-G/S-IGGG
(SEQ ID NO: 10)
AIT-G/S-LVGGVGLLLEVIVEVAYEEEEE
(SEQ ID NO: 11)
T-G/S-LVGGVGLLLEVIVEVAYEEEEE
(SEQ ID NO: 12)
G/S-LVGGVGLLLEVIVEVAYEEEEE
(SEQ ID NO: 13)
TLPAIV-G/S-IGGGGGELLLVIVEVLIYEEEEE
(SEQ ID NO: 14)
TLPAIT-G/S-LVGGVGLLLEVIVEVAYEEEEE
(SEQ ID NO: 15)
TLPAIV-G/S-IGGGVVLLLEVIVEVAYEEEEE

wherein the sequence may comprise 1, 2 or 3 substitutions of one amino acid at any position except the bold residue and the addition of 1 to 6 amino acids at the N-terminal or C-terminal end.

In a very particular aspect, the peptide comprises, consists essentially of or consists of an amino acid sequence selected from:

(SEQ ID NO: 52)
KGDWLPAITGLVGGVGLL
(SEQ ID NO: 53)
KGDWLPAIVSIGGGVVLL
(SEQ ID NO: 54)
KGDWIPALVGGGGGGGLL
(SEQ ID NO: 55)
KGDWLPALVSIGGGVGLL
(SEQ ID NO: 56)
KGDWIPALVGLGGGGGLL
(SEQ ID NO: 57)
KGDWLIAIVGIGGG
(SEQ ID NO: 58)
KGDWLPVIVGIGGG
(SEQ ID NO: 59)
KGDWLPAIVGIGGGGGLL
(SEQ ID NO: 60)
KGDWLPAIVGIGGGGGL
(SEQ ID NO: 61)
KGDWLPAIVGIGGGGG
(SEQ ID NO: 62)
KGDWLPAIVGIGGGG
(SEQ ID NO: 63)
KGDWLPAIVGIGGG
(SEQ ID NO: 16)
AITGLVGGVGLLLEVIVEVAYEEEEE;
(SEQ ID NO: 17)
TGLVGGVGLLLEVIVEVAYEEEEE;
(SEQ ID NO: 18)
GLVGGVGLLLEVIVEVAYEEEEE;
(SEQ ID NO: 19)
TLPAIVGIGGGGGELLLVIVEVLIYEEEEE;
(SEQ ID NO: 20)
TLPAITGLVGGVGLLLEVIVEVAYEEEEE;
(SEQ ID NO: 21)
TLPAIVSIGGGVVLLLEVIVEVAYEEEEE;
(SEQ ID NO: 97)
TLPAITGLVGGVGLLLEVIVEVAYEEE;
(SEQ ID NO: 98)
TLPAITGLVGGVGLLLEVIVEVAYEE;
(SEQ ID NO: 100)
TLPAITGLVGGVVLLLEVIVEVAYEEEEE;
(SEQ ID NO: 101)
TLPAITGLVGGVGLVLEVIVEVAYEEEEE;
(SEQ ID NO: 106)
_{d}E_{d}E_{d}E_{d}E_{d}E_{d}Y_{d}A_{d}V_{d}E_{d}V_{d}l_{d}V_{d}E_{d}L_{d}L_{d}LG_{d}VGG_{d}V_{d}LG_{d}T_{d}I_{d}A_{d}P_{d}L_{d}T;
(SEQ ID NO: 107)
TLPAITGLVGGVGLLLEVIVEVAYDD;
(SEQ ID NO: 108)
TLPAITGLVGGVGLLLEVIVEVAYDEDED;
(SEQ ID NO: 111)
_{d}TLPAITGLVGGVGLLLEVIVEVAYEEEEE;
(SEQ ID NO: 112)
TLPAITGLVGGVGLLLEVIVEVAYEEEEE;
or
(SEQ ID NO: 113)
TLPAITGLVGGVGLLVEVIVEVAYEEEEE;

wherein the sequence may comprise 1, 2 or 3 substitutions of one amino acid at any position except the bold residues and the addition of 1 to 6 amino acids at the N-terminal or C-terminal end.

In an embodiment, the peptide comprises, consists essentially of or consists of the amino acid sequence TLPAITGLVGGVGLLLEVIVEVAYEEEEE (SEQ ID NO: 20), TLPAITGLVGGVVLLLEVIVEVAYEEEEE (SEQ ID NO: 100), TLPAITGLVGGVGLLLEVIVEVAYDEDED (SEQ ID NO: 108) or TLPAITGLVGGVGLLVEVIVEVAYEEEEE (SEQ ID NO: 113).

The present disclosure also relates to a method for inhibiting the interaction between Neuropilin-1 and Plexin-A1 on a cell comprising contacting the cell with an effective amount of any peptide as defined herein.

The present disclosure also relates to the use of any peptide as defined herein for inhibiting the interaction between Neuropilin-1 and Plexin-A1 on a cell.

The present disclosure also relates to the use of any peptide as defined herein for the manufacture of a medicament for inhibiting the interaction between Neuropilin-1 and Plexin-A1 on a cell.

The present disclosure also relates to the peptide as defined herein for use for inhibiting the interaction between Neuropilin-1 and Plexin-A1 on a cell.

The present disclosure also relates to a method for inhibiting the anti-migratory and anti-differentiation effect of Sema3A in neuroglial cells (e.g., oligodendrocytes) comprising contacting the neuroglial cells with an effective amount of any peptide as defined herein.

The present disclosure also relates to the use of any peptide as defined herein for inhibiting anti-migratory and anti-differentiation effect of Sema3A in neuroglial cells (e.g., oligodendrocytes).

The present disclosure also relates to the use of any peptide as defined herein for the manufacture of a medicament for inhibiting the anti-migratory and anti-differentiation effect of Sema3A in neuroglial cells (e.g., oligodendrocytes).

The present disclosure also relates to the peptide as defined herein for use for inhibiting the anti-migratory and anti-differentiation effect of Sema3A in neuroglial cells (e.g., oligodendrocytes).

The present disclosure also relates to the peptide as defined herein for use for stimulating remyelination in a subject, such as a subject suffering from a demyelinating disease.

The present disclosure also relates to a pharmaceutical composition comprising any peptide as defined herein for use as a drug.

The present disclosure further relates to a pharmaceutical composition comprising any peptide as defined herein for the treatment of demyelinating diseases, especially demyelinating autoimmune or inflammatory diseases, including multiple sclerosis, transverse myelitis, neuromyelitis optica (Devic's disease), acute hemorrhagic leukoencephalitis, acute disseminated encephalomyelitis (ADEM), diffuse cerebral sclerosis of Schilder, adrenoleukodystrophy, Alexander disease, Canavan disease, Krabbe disease, Balo's disease, Charcot-Marie-Tooth disease (CMT), HIV encephalitis, HTLV-I Associated Myelopathy (HAM), Binswanger's disease (subcortical leukoencephalopathy and subcortical arteriosclerotic encephalopathy (SAE)) globoid cell leukodystrophy, metachromatic leukodystrophy, Pelizaeus-Merzbacher disease, progressive multifocal leukoencephalopathy, Marchiafava-Bignami disease, central pontine myelinolysis, polyradiculonueropathy including Guillain-Barre syndrome (GBS) and chronic inflammatory demyelinating polyradiculoneuropathy, demyelinating diseases caused by antineoplastic agents, carbon monoxide, vitamin B12 deficiency, mercury intoxication, alcohol/tobacco amblyopia, hypoxia or irradiation; or for the treatment of a disease or disorder associated with abnormal angiogenesis that would require a treatment reducing vascularization (e.g., hemangiomas, psoriasis, Kaposi's sarcoma, ocular neovascularization, Rheumatoid arthritis, endometriosis, or atherosclerosis) and/or for the treatment of cancer. It relates to the use of a pharmaceutical composition comprising any peptide as defined herein for the manufacture of a medicament for the treatment of demyelinating diseases, especially demyelinating autoimmune diseases, including the diseases as defined herein, or for the treatment of a disease or disorder associated with abnormal angiogenesis, and/or for the treatment of cancer. Finally, it relates to a method for the treatment of a demyelinating diseases, especially demyelinating autoimmune diseases, including the diseases as defined herein, of a disease or disorder associated with abnormal angiogenesis, or of cancer in a subject in need thereof, comprising administering a therapeutically effective amount of to a pharmaceutical composition comprising any peptide as defined herein to the subject.

In various aspects and embodiments, the present disclosure provides the following items 1 to 68:
1. A peptide comprising
   a first domain of the sequence X37-X5-X6-X7-G, and,
   (i) a second domain of the formula I: -(X26-X27-X28-X29-X30-X31-X32-X33-X34-X35-X36)-(CterpolyD/E) directly linked at carboxy-terminal end of the first domain;
(ii) a second domain of the formula II: -(X13-X14-X15-X16-X17-X18-X19-X20-X21-X22-X23-X24-X25)-(CterGp) directly linked at carboxy-terminal end of the first domain; or
   (iii) a second domain of the formula III: (NterGp)-(X8-X9-X10-X11-X12)-directly linked at amino-terminal end of the first domain;
   wherein
   X37 is Gly, L-Ser or D-Ser;
   X5, X6 and X7 are independently any amino acid;
   X8, X9, X10, X11 and X12 are independently any amino acid,
   X13, X14, X15, X16, X17, X18, X19, X20, X21, X22, X23, X24 and X25 are independently any amino acid, wherein no more than 2 amino acids among X13, X14, X15, X16, X17, X18, X19, X20, X21, X22, X23, X24 and X25 are a charged amino acid;
   X26, X27, X28, X29, X30, X31, X32, X33, X34, X35 and X36 are independently any amino acid, wherein no more than 2 amino acids among X26, X27, X28, X29, X30, X31, X32, X33, X34, X35 are a charged amino acid;
   CterpolyD/E is a group of 4-10 amino acids including at least 2 negatively charged amino acids
   NterGp is a group of 3-5 amino acids including at least 2 charged amino acids, and
   CterGp is a group of 3-5 amino acids including at least 3 positively charged amino acids,
   or a retro or retroinverso form thereof,
   or a pharmaceutically acceptable salt thereof,
      wherein the peptide does not comprise or consist of the sequence TLPAIVGIGGGGGLLLLVIVAVLIAYKRK (SEQ ID NO: 1).
2. The peptide of item 1, wherein X5, X6 and X7 are aliphatic uncharged amino acids.
3. The peptide of item 2, wherein X5, X6 and X7 are each independently Gly, D or L-Ala, D or L-Val, D or L-Leu and D or L-Ile.
4. The peptide of item 3, wherein X5 is D-Leu, L-Leu, D-Ile or L-Ile; X6 is Gly, D-Val or L-Val; and/or X7 is Gly.
5. The peptide of item 4, wherein X5 is L-Leu and/or X6 is L-Val.
6. The peptide of any one of items 1 to 5, wherein X6 is Gly, D-Val or L-Val.
7. The peptide of any one of items 1 to 6, wherein X37 is Gly.
8. The peptide of any one of items 1 to 7, wherein X26 is an aliphatic uncharged amino acid; X27 is an aliphatic uncharged amino acid; X28 is an aliphatic uncharged amino acid; X29 is an aliphatic uncharged amino acid; X30 is an aliphatic uncharged amino acid; X31 is a negatively charged amino acid; X32 is an aliphatic uncharged amino acid; X33 is an aliphatic uncharged amino acid; X34 is an aliphatic uncharged amino acid; X35 is a negatively charged amino acid; and/or X36 is an aliphatic uncharged amino acid.
9. The peptide of item 8, wherein X26 is Gly, D-Val or L-Val; X27 is Gly, D-Val or L-Val; X28 is D or L-Ala, D- or L-Val, D- or L-Leu, or D- or L-Ile; X29 is D- or L-Ala, D- or L-Val, D- or L-Leu, or D- or L-Ile; X30 is D- or L-Ala, D- or L-Val, D- or L-Leu, or D- or L-Ile; X31 is D or L-Glu; X32 is D- or L-Ala, D- or L-Val, D- or L-Leu, or D- or L-Ile; X33 is D- or L-Ala, D- or L-Val, D- or L-Leu, or D- or L-Ile; X34 is D- or L-Ala, D- or L-Val, D- or L-Leu, or D- or L-Ile; X35 is D or L-Glu; and/or X36 is D- or L-Ala, D- or L-Val, D- or L-Leu, or D- or L-Ile.
10. The peptide of item 9, wherein X26 is L-Val; X27 is Gly; X28 is L-Leu; X29 is L-Leu; X30 is L-Leu; X31 is L-Glu; X32 is L-Val; X33 is L-Ile; X34 is L-Val; X35 is L-Glu; and/or X36 is L-Val.
11. The peptide of any one of items 1 to 10, wherein X26-X27-X28-X29-X30-X31-X32-X33-X34-X35-X36 is VGLLLEVIVEV (SEQ ID NO: 91), GGELLLVIVE (SEQ ID NO: 92), VVLLLEVIVEV (SEQ ID NO: 93), VGLLVEVIVEV (SEQ ID NO:117), or VGLVLEVIVEV (SEQ ID NO:118).
12. The peptide of any one of items 1 to 11, wherein CterpolyD/E comprises a sequence of -X″1-X″2-Z or -X″3-X″3bis-X″2-Z,
   wherein
   X″1 is a small amino acid,
   X″2 is an aromatic amino acid,
   X″3 and X″3bis are independently long aliphatic amino acids
   Z is from 2 to 10 D/L-Asp and/or D/L-Glu residues.
13. The peptide of item 12, wherein X″1 is D- or L-Ala.
14. The peptide of item 12 or 13, wherein X″3 and X″3bis are independently D- or L-Leu, or D- or L-Ile.
15. The peptide of item 14, wherein X″3 is D- or L-Leu, and X″3bis is D- or L-Ile.
16. The peptide of any one of items 12 to 15, wherein Z is from 3 to 6 D/L-Asp and/or D/L-Glu residues.
17. The peptide of any one of items 12 to 16, wherein Z is from 3 to 5 L-Glu residues.
18. The peptide of any one of items 1 to 17, wherein the peptide comprises a second domain of formula I or II, and further comprises a third domain of 1 to 10 amino acids linked at the amino-terminal end of the first domain.
19. The peptide of item 18, wherein the third domain comprises from 1 to 6 amino acids.
20. The peptide of item 19, wherein the third domain is of the formula IV: X38-X39-X40-X41-X42-X43, wherein
   X38 is D-Thr or L-Thr, or is absent;
   X39 is D- or L-Leu, D- or L-Ile, or is absent;
   X40 is Pro, D- or L-Ile, or is absent;
   X41 is D- or L-Ala, D- or L-Val, or is absent;
   X42 is D- or L-Leu, D- or L-Ile, or is absent; and
   X43 is D- or L-Val, D-Thr or L-Thr.
21. The peptide of any one of items 1 to 20, which comprises 35 amino acid or less.
22. The peptide of any one of items 1 to 21, which comprises 30 amino acid or less.
23. The peptide of any one of items 1 to 22, which comprises one of the following sequences:

(SEQ ID NO: 16)
AITGLVGGVGLLLEVIVEVAYEEEEE;
(SEQ ID NO: 17)
TGLVGGVGLLLEVIVEVAYEEEEE;
(SEQ ID NO: 18)
GLVGGVGLLLEVIVEVAYEEEEE;
(SEQ ID NO: 19)
TLPAIVGIGGGGGELLLVIVEVLIYEEEEE;
(SEQ ID NO: 20)
TLPAITGLVGGVGLLLEVIVEVAYEEEEE;
(SEQ ID NO: 21)
TLPAIVSIGGGVVLLLEVIVEVAYEEEEE;
(SEQ ID NO: 97)
TLPAITGLVGGVGLLLEVIVEVAYEEE;
(SEQ ID NO: 98)
TLPAITGLVGGVGLLLEVIVEVAYEE;
(SEQ ID NO: 100)
TLPAITGLVGGVVLLLEVIVEVAYEEEEE;
(SEQ ID NO: 101)
TLPAITGLVGGVGLVLEVIVEVAYEEEEE;
(SEQ ID NO: 106)
_{d}E_{d}E_{d}E_{d}E_{d}E_{d}Y_{d}A_{d}V_{d}E_{d}V_{d}l_{d}V_{d}E_{d}L_{d}L_{d}LG_{d}VGG_{d}V_{d}LG_{d}T_{d}I_{d}A_{d}P_{d}L_{d}T;
(SEQ ID NO: 107)
TLPAITGLVGGVGLLLEVIVEVAYDD;
(SEQ ID NO: 108)
TLPAITGLVGGVGLLLEVIVEVAYDEDED;
(SEQ ID NO: 111)
_{d}TLPAITGLVGGVGLLLEVIVEVAYEEEEE;
(SEQ ID NO: 112)
TLPAITGLVGGVGLLLEVIVEVAYEEEEE;
or
(SEQ ID NO: 113)
TLPAITGLVGGVGLLVEVIVEVAYEEEEE;

24. The peptide of item 23, which comprises one of the following sequences:

(SEQ ID NO: 20)
TLPAITGLVGGVGLLLEVIVEVAYEEEEE;
(SEQ ID NO: 100)
TLPAITGLVGGVVLLLEVIVEVAYEEEEE;
(SEQ ID NO: 108)
TLPAITGLVGGVGLLLEVIVEVAYDEDED;
or
(SEQ ID NO: 113)
TLPAITGLVGGVGLLVEVIVEVAYEEEEE.

25. The peptide of any one of items 1 to 7, wherein X8 is an aliphatic residue; X9 is Pro, or D- or L-Ile; X10 is an aliphatic residue; X11 is an aliphatic residue; and/or X12 is an aliphatic residue.
26. The peptide of item 25, wherein X8 is D- or L-Leu, or D- or L-Ile; X9 is Pro; X10 is D- or L-Ala, or D- or L-Val; X11 is D- or L-Leu, or D- or L-Ile; and/or X12 is D- or L-Thr, or D- or L-Val.
27. The peptide of item 26, wherein X8-X9-X10-X11-X12 is LPAIT (SEQ ID NO:82), LPAIV (SEQ ID NO:83), IPALV (SEQ ID NO:84), LPALV (SEQ ID NO:85), LIAIV (SEQ ID NO:86) or LPVIV (SEQ ID NO:87).
28. The peptide of any one of items 1 to 7 and 25 to 27, wherein NterGp comprises one positively charged amino acid and one negatively charged amino acid.
29. The peptide of item 28, wherein NterGp has a sequence of 4 amino acids of the formula V: X1-X2-X3-X4, wherein X1 is a positively charged residue and X3 is a negatively charged residue, X2 is a small amino acid, and X4 being an aromatic amino acid.
30. The peptide of item 29, wherein X1 is D- or L-Lys, X2 is Gly, X3 is D- or L-Asp; and/or X4 is D- or L-Trp.
31. The peptide of item 30, wherein NterGp is KGDW (SEQ ID NO:116).
32. The peptide of any one of items 1 to 7 and 25 to 31, wherein the second domain is of the formula III, and wherein the peptide further comprises a third domain of 1 to 10 amino acids linked at the carboxy-terminal end of the first domain.
33. The peptide of item 32, wherein the third domain is of the formula VI: X26-X27-X28-X29-X30-X31-X32-X33-X34-X35, wherein X26, X27, X28, X29, X30, X31, X32, X33, X34 and X35 are as defined in any one of items 1 and 8 to 11.
34. The peptide of any one of items 1 to 7 and 25 to 33, which comprises one of the following sequences:

(SEQ ID NO: 52)
KGDWLPAITGLVGGVGLL;
(SEQ ID NO: 53)
KGDWLPAIVSIGGGVVLL;
(SEQ ID NO: 54)
KGDWIPALVGGGGGGGLL;
(SEQ ID NO: 55)
KGDWLPALVSIGGGVGLL;
(SEQ ID NO: 56)
KGDWIPALVGLGGGGGLL;
(SEQ ID NO: 57)
KGDWLIAIVGIGGG;
(SEQ ID NO: 58)
KGDWLPVIVGIGGG;
(SEQ ID NO: 59)
KGDWLPAIVGIGGGGGLL;
(SEQ ID NO: 60)
KGDWLPAIVGIGGGGGL;
(SEQ ID NO: 61)
KGDWLPAIVGIGGGGG;
(SEQ ID NO: 62)
KGDWLPAIVGIGGGG;

or
35. The peptide of any one of items 1 to 7, wherein X13 is an aliphatic and/or small residue; X14 is an aliphatic and/or small residue; X15 is an aliphatic residue or a negatively charged residue; X16 is an aliphatic residue; X17 is an aliphatic residue; X18 is an aliphatic residue or a negatively charged residue; X19 is an aliphatic residue; X20 is an aliphatic residue; X21 is an aliphatic residue; X22 is a small residue or a negatively charged residue; X23 is an aliphatic residue; X24 is an aliphatic residue; and/or X25 is an aliphatic residue.
36. The peptide of item 35, wherein X13 is Gly or D- or L-Val; X14 is Gly or D- or L-Val; X15 is D- or L-Ile, or D- or L-Leu; X16 is D- or L-Ile, or D- or L-Leu; X17 is D- or L-Ile, or D- or L-Leu; X18 is D- or L-Ile, or D- or L-Leu; X19 is D- or L-Val; X20 is D- or L-Ile, or D- or L-Leu; X21 is D- or L-Val; X22 is D- or L-Ala; X23 is D- or L-Val; X24 is D- or L-Ile, or D- or L-Leu; and/or X25 is D- or L-Ile, or D- or L-Leu.
37. The peptide of item 35, wherein X13-X14-X15-X16-X17-X18-X19-X20-X21-X22-X23-X24-X25 is GGLLLLVIVAVLI (SEQ ID NO:88).
38. The peptide of any one of items 1 to 7 and 35 to 37, wherein CterGp comprises a mixture of Lys and Arg residues.
39. The peptide of item 38, wherein CterGp comprises one of the following sequences:

(SEQ ID NO: 76)
AYKRK,
(SEQ ID NO: 77)
AYKKR,
(SEQ ID NO: 78)
AYKRR,
(SEQ ID NO: 79)
AYRRK
or
(SEQ ID NO: 80)
AYRKK

40. A pharmaceutical composition comprising the peptide, retro or retroinverso form thereof, or pharmaceutically acceptable salt thereof according to any one of items 1 to 39, and a pharmaceutically acceptable carrier.
41. The peptide, retro or retroinverso form thereof, or pharmaceutically acceptable salt thereof according to any one of items 1 to 39, or the pharmaceutical composition according to item 40, for use as a medicament.
42. The peptide, retro or retroinverso form thereof, or pharmaceutically acceptable salt thereof according to any one of items 1 to 39, or the pharmaceutical composition according to item 40, for use in the treatment of a demyelinating disease in a subject.
43. The peptide, retro or retroinverso form thereof, pharmaceutically acceptable salt thereof, or composition for use according to item 42, wherein the demyelinating disease is a demyelinating autoimmune disease
44. The peptide, retro or retroinverso form thereof, pharmaceutically acceptable salt thereof, or composition for use according to item 42 or 43, wherein the demyelinating disease is multiple sclerosis, transverse myelitis, neuromyelitis optica (Devic's disease), acute hemorrhagic leukoencephalitis, acute disseminated encephalomyelitis (ADEM), diffuse cerebral sclerosis of Schilder, adrenoleukodystrophy, Alexander disease, Canavan disease, Krabbe disease, Balo's disease, Charcot-Marie-Tooth disease (CMT), HIV encephalitis, HTLV-I Associated Myelopathy (HAM), Binswanger's disease (subcortical leukoencephalopathy and subcortical arteriosclerotic encephalopathy (SAE)), globoid cell leukodystrophy, metachromatic leukodystrophy, Pelizaeus-Merzbacher disease, progressive multifocal leukoencephalopathy, Marchiafava-Bignami disease, central pontine myelinolysis, polyradiculonueropathy, or demyelinating diseases caused by antineoplastic agents, carbon monoxide, vitamin B12 deficiency, mercury intoxication, alcohol or tobacco amblyopia, hypoxia or irradiation.
45. The peptide, retro or retroinverso form thereof, pharmaceutically acceptable salt thereof, or composition for use according to item 44, wherein the polyradiculoneuropathy is Guillain-Barre syndrome (GBS) chronic or inflammatory demyelinating polyradiculoneuropathy.
46. The peptide, retro or retroinverso form thereof, pharmaceutically acceptable salt thereof, or composition for use according to item 44, wherein the demyelinating disease is multiple sclerosis.
47. The peptide, retro or retroinverso form thereof, or pharmaceutically acceptable salt thereof according to any one of items 1 to 39, or the pharmaceutical composition according to item 40, for use in the treatment of a disease or disorder associated with abnormal angiogenesis.
48. The peptide, retro or retroinverso form thereof, pharmaceutically acceptable salt thereof, or composition for use according to item 47, wherein the disease or disorder associated with abnormal angiogenesis is cancer, hemangiomas, psoriasis, Kaposi's sarcoma, ocular neovascularization, rheumatoid arthritis, endometriosis, or atherosclerosis.
49. The peptide, retro or retroinverso form thereof, pharmaceutically acceptable salt thereof, or composition for use according to any one of items 42 to 48, wherein the peptide, retro or retroinverso form thereof, pharmaceutically acceptable salt thereof, or composition is for use in combination with one or more additional therapeutic agents.
50. The peptide, retro or retroinverso form thereof, pharmaceutically acceptable salt thereof, or composition for use according to item 49, wherein the one or more additional therapeutic agents comprise fingolimod.
51. Use of the peptide, retro or retroinverso form thereof, or pharmaceutically acceptable salt thereof according to any one of items 1 to 39, or the pharmaceutical composition according to item 40, for the manufacture of a medicament for the treatment of a demyelinating disease in a subject.
52. The use according to item 51, wherein the demyelinating disease is a demyelinating autoimmune disease
53. The use according to item 51 or 52, wherein the demyelinating disease is multiple sclerosis, transverse myelitis, neuromyelitis optica (Devic's disease), acute hemorrhagic leukoencephalitis, acute disseminated encephalomyelitis (ADEM), diffuse cerebral sclerosis of Schilder, adrenoleukodystrophy, Alexander disease, Canavan disease, Krabbe disease, Balo's disease, Charcot-Marie-Tooth disease (CMT), HIV encephalitis, HTLV-I Associated Myelopathy (HAM), Binswanger's disease (subcortical leukoencephalopathy and subcortical arteriosclerotic encephalopathy (SAE)), globoid cell leukodystrophy, metachromatic leukodystrophy, Pelizaeus-Merzbacher disease, progressive multifocal leukoencephalopathy, Marchiafava-Bignami disease, central pontine myelinolysis, polyradiculonueropathy, or demyelinating diseases caused by antineoplastic agents, carbon monoxide, vitamin B12 deficiency, mercury intoxication, alcohol or tobacco amblyopia, hypoxia or irradiation.
54. The use according to item 53, wherein the polyradiculoneuropathy is Guillain-Barre syndrome (GBS) or chronic inflammatory demyelinating polyradiculoneuropathy.
55. The use according to item 53, wherein the demyelinating disease is multiple sclerosis.
56. Use of the peptide, retro or retroinverso form thereof, or pharmaceutically acceptable salt thereof according to any one of items 1 to 39, or the pharmaceutical composition according to item 40, for the manufacture of a medicament for the treatment of a disease or disorder associated with abnormal angiogenesis.
57. The use according to item 56, wherein the disease or disorder associated with abnormal angiogenesis is cancer, hemangiomas, psoriasis, Kaposi's sarcoma, ocular neovascularization, rheumatoid arthritis, endometriosis, or atherosclerosis.
58. The use according to any one of items 51 to 57, wherein the medicament is for use in combination with one or more additional therapeutic agents.
59. The use according to item 58, wherein the one or more additional therapeutic agents comprise fingolimod.
60. A method for treating a demyelinating disease in a subject in need thereof, the method comprising administering to the subject an effective amount of the peptide, retro or retroinverso form thereof, or pharmaceutically acceptable salt thereof according to any one of items 1 to 39, or the pharmaceutical composition according to item 40.
61. The method according to item 60, wherein the demyelinating disease is a demyelinating autoimmune disease
62. The method according to item 60 or 61, wherein the demyelinating disease is multiple sclerosis, transverse myelitis, neuromyelitis optica (Devic's disease), acute hemorrhagic leukoencephalitis, acute disseminated encephalomyelitis (ADEM), diffuse cerebral sclerosis of Schilder, adrenoleukodystrophy, Alexander disease, Canavan disease, Krabbe disease, Balo's disease, Charcot-Marie-Tooth disease (CMT), HIV encephalitis, HTLV-I Associated Myelopathy (HAM), Binswanger's disease (subcortical leukoencephalopathy and subcortical arteriosclerotic encephalopathy (SAE)), globoid cell leukodystrophy, metachromatic leukodystrophy, Pelizaeus-Merzbacher disease, progressive multifocal leukoencephalopathy, Marchiafava-Bignami disease, central pontine myelinolysis, polyradiculonueropathy, or demyelinating diseases caused by antineoplastic agents, carbon monoxide, vitamin B12 deficiency, mercury intoxication, alcohol or tobacco amblyopia, hypoxia or irradiation.
63. The method according to item 62, wherein the polyradiculoneuropathy is Guillain-Barre syndrome (GBS) or chronic inflammatory demyelinating polyradiculoneuropathy.
64. The method according to item 62, wherein the demyelinating disease is multiple sclerosis.
65. A method for treating a disease or disorder associated with abnormal angiogenesis in a subject in need thereof, the method comprising administering to the subject an effective amount of the peptide, retro or retroinverso form thereof, or pharmaceutically acceptable salt thereof according to any one of items 1 to 39, or the pharmaceutical composition according to item 40.
66. The method according to item 65, wherein the disease or disorder associated with abnormal angiogenesis is cancer, hemangiomas, psoriasis, Kaposi's sarcoma, ocular neovascularization, rheumatoid arthritis, endometriosis, or atherosclerosis.
67. The method according to any one of items 60 to 66, wherein the peptide, retro or retroinverso form thereof, pharmaceutically acceptable salt thereof, or composition is for use in combination with one or more additional therapeutic agents.
68. The method according to item 67, wherein the one or more additional therapeutic agents comprise fingolimod.

### BRIEF DESCRIPTION OF THE DRAWINGS

FIG. 1. Membrane targeting peptides block NRP1-PlexinA1 receptor dimerization. Interfering activity of membrane targeting peptides drives to decrease of interaction between Nrp1 and PlexinA1 measured by proximity ligation assay.

FIGS. 1A-B: Quantification of NRP1-PlexinA1 interaction per Oli-neu cells treated with different doses of GUNGNIR or MIMMING as measured by proximity ligation assay (data are presented as mean±SEM, Kruskal-Wallis test, ***P<0.0001 **P<0.01).

FIGS. 2A-G: Membrane targeting peptides counteract Sema3A negative effect on migration of the oligodendrocyte cell line Oli-neu. FIG. 2A: Peptides with different synthetic sequences bearing G-X-X-X-G motif rescue migration (n=3 except SKOLL and HATI n=1). FIG. 2B: Peptides with G/S-X-X-X-G motif, but not those with G-X-X-X-S or S-X-X-X-A motifs, rescue migration of Oli-neu cells treated with Sema3A (n=3 excepted SKIRNIR n=1). FIG. 2C: Peptides with deletions next to G-X-X-X-G motif on N-ter or C-ter side rescue migration of Oli-neu cells treated with Sema3A, but deletion of one G of the motif abrogates peptide activity (n=1). FIG. 2D: N-ter shortening between (NterGp) and G-X-X-X-G motif impairs peptide efficiency (n=3 excepted BROKK and EITRI n=1). FIG. 2E: Among other peptides sequences with (CterpolyD/E), RATI and GUNGNIR and MIMMING rescue migration (n=3). FIG. 2F: GUNGNIR IC₅₀ calculation at 1.2 nM (n=3). FIG. 2G: Comparison of MTP-PlexA1 (SEQ D NO: 1) and a retro analog thereof (ODIN). Data are presented as mean±SEM when n=3 independent experiments, ANOVA and Bonferroni's multiple comparison test relative to vehicle condition **P<0.01, ***P<0.0001.

FIG. 3: Analysis of membrane targeting peptides biodistribution. Proportion of bioluminescence of GUNGNIR-Cy5 in different organs 4 h after intraperitoneal injection of indicated doses (μg/kg).

FIGS. 4A-B: GUNGNIR reduces EAE severity. FIG. 4A: GUNGNIR at 10 mg/kg reduces EAE clinical score following PLP immunization. FIG. 4B: GUNGNIR at 10 mg/kg reduces EAE clinical score following MOG immunization. Data are presented as mean±SEM, n=7 (FIG. 4A) and n=10 (FIG. 4B) Non-linear regressions (bell shape) are plotted and used for statistical significance P<0.0001 (FIG. 4A) P=0.0002 (FIG. 4B).

FIG. 5A: Membrane targeting peptides inhibit angiogenesis. Tubulogenesis was measured by counting tube-like structure intersections of HUVECs. A decrease in the number of tube-like structure intersections is indicative of an inhibition of angiogenesis by the MTPs.

FIG. 5B show the results of the MTT (3-[4,5-dimethylthiazol-2-yl]-2,5 diphenyl tetrazolium bromide) toxicity assay on HUVEC cells.

FIGS. 6A-B: Results of the Gait analysis after 6 days remyelination showing the two parameters that were found to be different between vehicle and 100 μg/kg GUNGNIR groups. FIG. 6A: Propel 6 days right hind. FIG. 6B: Min dA/dT 6 days left hind.

FIGS. 7A-F: Results of the Gait analysis after 11 days remyelination showing the five parameters that were found to be different between vehicle and 10 or 100 μg/kg GUNGNIR groups. FIG. 7A: Min dA/dT 11 days right fore. FIG. 7B: Stance factor 11 days left fore. FIG. 7C: Propel 11 days right hind. FIG. 7D: Stance 11 days right hind. FIG. 7E: Paw area variability at Peak stance 11 days left hind.

FIGS. 8A-B: Results of histology experiments showing the staining for the major myelin protein PLP (FIG. 8A) and Luxol-fast-blue (LFB) staining of myelin phospholipids (FIG. 8B) in brain tissues from mice subjected to cuprizone-induced demyelination.

FIGS. 9A-G: Results of body weight in mice subjected to cuprizone-induced demyelination. FIG. 9A: Body weight before the initiation of the GUNGNIR treatment (i.e., at day 21). FIG. 9B: Body weight at the start of the experiment. FIG. 9C: Body weight at the end of the experiment. FIG. 9D: Weight loss at day 21 in the 6-day remyelination group. FIG. 9E: Weight loss at day 21 in the 11-day remyelination group. FIG. 9F: Weight loss at week 5 in the 6-day remyelination group. FIG. 9G: Weight loss at week 5 in the 11-day remyelination group.

FIG. 10: Weight monitoring over the course of the experiment in the various groups of EAE-PLP mice.

FIGS. 11A-F: Clinical scores of disease severity in the various groups of EAE-PLP mice. FIG. 11A: clinical scores over the course of the experiment. FIGS. 11B-F: clinical scores at day 22, 26, 28, 38 and 39, respectively.

FIG. 12: Weight monitoring over the course of the experiment in the various groups of EAE-MOG mice.

FIGS. 13A-O: Clinical scores of disease severity in the various groups of EAE-MOG mice. FIG. 13A: clinical scores over the course of the experiment. FIGS. 13B-O: clinical scores at day 17, 18, 19, 20, 21, 22, 23, 24, 25, 26, 27, 28, 39 and 30, respectively.

### DETAILED DESCRIPTION OF THE INVENTION

The use of any and all examples, or exemplary language (“e.g.”, “such as”, etc.) provided herein, is intended merely to better illustrate the invention and does not pose a limitation on the scope of the invention unless otherwise claimed.

Herein, the term “about” has its ordinary meaning. The term “about” is used to indicate that a value includes an inherent variation of error for the device or the method being employed to determine the value, or encompass values close to the recited values, for example within 10% or 5% of the recited values (or range of values).

Unless otherwise defined, all technical and scientific terms used herein have the same meaning as commonly understood by one of ordinary skill in the art to which this invention belongs.

Any and all combinations and subcombinations of the embodiments and features disclosed herein are encompassed by the present invention.

The present disclosure provides new antagonistic peptides capable of inhibiting the interaction between Neuropilin-1/Plexin-A1 and/or the inhibitory effect of Sema3A on migration and relates to a pharmaceutical composition comprising such antagonistic peptides and to the use thereof as a drug.

### Definition

Plexin-A1 is a protein encoded by the PLXNA1 gene. It is described in several databases, namely UniProt ID No Q9UIW2; HGNG ID No 9099. Reference sequences are disclosed in Genbank under NM_032242.3 for mRNA and NP_115618.3 for protein.

Neuropilin-1 is a protein encoded by the NRP1 gene. It is described in several databases, namely UniProt ID No 014786; HGNG ID No 8004. Reference sequences are disclosed in Genbank under NM_001330068.1 for mRNA and NP_001316997.1 for protein.

“consists of,” “consists essentially of” or “substantially comprises”: The description herein of any aspect or embodiment of the disclosure using terms such as reference to an element or elements is intended to provide support for a similar aspect or embodiment of the invention that “consists of,” “consists essentially of” or “substantially comprises” that particular element or elements, unless otherwise stated or clearly contradicted by context. For instance, a peptide or protein described herein as comprising a particular sequence should be understood as also describing a peptide or protein consisting of that sequence, unless otherwise stated or clearly contradicted by context. By “consists essentially of” is intended that the peptide or protein consists of that sequence, but it may also include 1, 2, 3, 4, 5, 6, 7, 8, 9 or 10 substitutions, additions, deletions or a mixture thereof, particularly 1, 2, 3, 4, or 5 substitutions, additions, deletions or a mixture thereof, more particularly 1, 2 or 3 substitutions, additions, deletions or a mixture thereof. In particular, by “essentially consist in”, it may be intended that the peptide may include 1, 2, 3, 4, 5, 6, 7, 8, 9 or 10 additional amino acids at the N and/or C-terminal end, particularly 1, 2, 3, 4, or 5 additional amino acids, more particularly 1, 2 or 3 additional amino acids, and/or 1, 2 or 3 substitutions, deletions, additions, or a mixture thereof. In a particular aspect, the sequence has no more than 2 or 3 substitutions. Preferably, the number of substitutions, additions, deletions or a mixture thereof depends on the length of the sequence. For instance, the percentage of substitutions, deletions, additions, or a mixture thereof may be no more than 30%, preferably no more than 25%, more preferably no more than 8 or 10%.

As used herein, the term “substitution” refers to the exchange of a single amino acid by another in a peptide sequence. As used herein, the term “deletion” refers to the removal of a single amino acid in a peptide sequence. As used herein, the term “insertion” or “addition” are equivalent and refer to the addition of a single amino acid in a peptide sequence.

By “substitutions, additions, deletions” is intended a substitution, addition, deletion of one amino acid. Then, when it is referred to “1, 2, 3, 4, 5, 6, 7, 8, 9 or 10 substitutions, additions, deletions or a mixture thereof”, “1, 2, 3, 4, or 5 substitutions, additions, deletions or a mixture thereof” or “1, 2 or 3 substitutions, deletions, additions, or a mixture thereof”, it means respectively “1, 2, 3, 4, 5, 6, 7, 8, 9 or 10 modification(s) of an amino acid selected from substitutions, additions, deletions and a mixture thereof”, “1, 2, 3, 4, or 5 modification(s) of an amino acid selected from substitutions, additions, deletions or a mixture thereof” or “1, 2 or 3 modification(s) of an amino acid selected from substitutions, deletions, additions, or a mixture thereof”. “1, 2, 3, 4, or 5 substitutions, additions, deletions or a mixture thereof” also means “from 1 to 5 substitutions, additions, deletions or a mixture thereof”. “1, 2, or 3 substitutions, additions, deletions or a mixture thereof” also means “from 1 to 3 substitutions, additions, deletions or a mixture thereof”.

In the peptide sequences disclosed herein, the amino acids are represented by their one letter code or three letter code according to the following nomenclature: A: Ala, alanine; C: Cys, cysteine; D: Asp, aspartic acid; E: Glu, glutamic acid; F: Phe, phenylalanine; G: Gly, glycine; H: His, histidine; I: Ile, isoleucine; K: Lys, lysine; L: Leu, leucine; M: Met, methionine; N: Asn, asparagine; P: Pro, proline; Q: Gln, glutamine; R: Arg, arginine; S: Ser, serine; T: Thr, threonine; V: Val, valine; W: Trp, tryptophane and Y: Tyr, tyrosine.

A conservative substitution is the replacement of a given amino acid residue by another residue having a side chain (“R-group”) with similar chemical properties (e.g., charge, bulk and/or hydrophobicity). In general, a conservative amino acid substitution will not substantially change the functional properties of a protein. Conservative substitutions and the corresponding rules are well-described in the state of the art. For instance, conservative substitutions can be defined by substitutions within the groups of amino acids reflected in the following tables:

TABLE A
Amino Acid Residue

| Amino Acid groups | Amino Acid Residues |
|---|---|
| Acidic Residues | ASP and GLU |
| Basic Residues | LYS, ARG, and HIS |
| Hydrophilic Uncharged Residues | SER, THR, ASN, and GLN |
| Aliphatic Uncharged Residues | GLY, ALA, VAL, LEU, and ILE |
| Non-polar Uncharged Residues | CYS, MET, and PRO |
| Aromatic Residues | PHE, TYR, and TRP |

TABLE B
Alternative Conservative Amino Acid Residue Substitution Groups

| | | | |
|---|---|---|---|
| 1 | Alanine (A) | Serine (S) | Threonine (T) |
| 2 | Aspartic acid (D) | Glutamic acid (E) | |
| 3 | Asparagine (N) | Glutamine (Q) | |
| 4 | Arginine (R) | Lysine (K) | |
| 5 | Isoleucine (I) | Leucine (L) | Methionine (M) |
| 6 | Phenylalanine (F) | Tyrosine (Y) | Tryptophan (W) |

TABLE C
Further Alternative Physical and Functional
Classifications of Amino Acid Residues

| | |
|---|---|
| Alcohol group-containing residues | S and T |
| Aliphatic residues | I, L, V, and M |
| Cycloalkenyl-associated residues | F, H, W, and Y |
| Hydrophobic residues | A, C, F, G, H, I, L, M, R, T, V, W, |
| | and Y |
| Negatively charged residues | D and E |
| Polar residues | C, D, E, H, K, N, Q, R, S, and T |
| Small residues | A, C, D, G, N, P, S, T, and V |
| Very small residues | A, G, and S |
| Residues involved in turn | A, C, D, E, G, H, K, N, Q, R, S, P, |
| formation | and T |
| Flexible residues | E, Q, T, K, S, G, P, D, E, and R |

The peptides described herein may comprise L- and D-isomers of the naturally occurring amino acids as well as other amino acids (e.g., naturally-occurring amino acids, non-naturally-occurring amino acids, amino acids which are not encoded by nucleic acid sequences, etc.) used in peptide chemistry to prepare synthetic analogs of peptides. Examples of naturally-occurring amino acids are glycine, alanine, valine, leucine, isoleucine, serine, threonine, etc. Other amino acids include for example non-genetically encoded forms of amino acids, as well as a conservative substitution of an L-amino acid. Naturally-occurring non-genetically encoded amino acids include, for example, beta-alanine, 3-amino-propionic acid, 2,3-diamino propionic acid, alpha-aminoisobutyric acid (Aib), 4-amino-butyric acid, N-methylglycine (sarcosine), hydroxyproline, ornithine (e.g., L-ornithine), citrulline, t-butylalanine, t-butylglycine, N-methylisoleucine, phenylglycine, cyclohexylalanine, norleucine (Nle), norvaline, 2-napthylalanine, pyridylalanine, 3-benzothienyl alanine, 4-chlorophenylalanine, 2-fluorophenylalanine, 3-fluorophenylalanine, 4-fluorophenylalanine, penicillamine, 1,2,3,4-tetrahydro-isoquinoline-3-carboxylix acid, beta-2-thienylalanine, methionine sulfoxide, L-homoarginine (Hoarg), N-acetyl lysine, 2-amino butyric acid, 2-amino butyric acid, 2,4,-diaminobutyric acid (D- or L-), p-aminophenylalanine, N-methylvaline, homocysteine, homoserine (HoSer), cysteic acid, epsilon-amino hexanoic acid, delta-amino valeric acid, or 2,3-diaminobutyric acid (D- or L-), etc. These amino acids are well known in the art of biochemistry/peptide chemistry.

The peptides described herein may comprise all L-amino acids, all D-amino acids or a mixture of L- and D-amino acids. In an embodiment, the peptides comprise only L-amino acids.

In addition to the substitutions outlined above, synthetic amino acids providing similar side chain functionality can also be introduced into the peptide. For example, aromatic amino acids may be replaced with D- or L-naphthylalanine, D- or L-phenylglycine, D- or L-2-thienylalanine, D- or L-1-, 2-, 3-, or 4-pyrenylalanine, D- or L-3-thienylalanine, D- or L-(2-pyridinyl)-alanine, D- or L-(3-pyridinyl)-alanine, D- or L-(2-pyrazinyl)-alanine, D- or L-p-cyano-phenylalanine, D- or L-(4-isopropyl)-phenylglycine, D- or L-(trifluoromethyl)-phenylglycine, D- or L-(trifluoromethyl)-phenylalanine, D- or L-p-fluorophenylalanine, D- or L-p-biphenylalanine, D- or L-p-methoxybiphenylalanine, D- or L-2-indole (alkyl) alanines, and D- or L-alkylalanines wherein the alkyl group is selected from the group consisting of substituted or unsubstituted methyl, ethyl, propyl, hexyl, butyl, pentyl, isopropyl, iso-butyl, and iso-pentyl.

Analogs of lysine comprising a primary amine in their side chain include ornithine, homolysine, 2,3-diaminoproprionic acid (Dap), and 2,4-diaminobutyric acid (Dab).

Analogs of histidine include, for example, those described in Ikeda et al., Protein Eng. (2003) 16 (9): 699-706 (e.g., β-(1,2,3-triazol-4-yl)-DL-alanine), those described in Stefanucci et al., Int. J. Mol. Sci. 2011, 12(5), 2853-2890 (aza-histidine, homo-histidine, β2-homo-histidine, β3-homo-histidine, Nor-histidine), N-imidazolyl alanine, methyl histidine, dimethyl histidine, C-triazolyl alanine, histidine methyl ester, histidinol, and histidinamide.

Analogs of tryptophan include, for example, naphthylalanines, indenylalanines, 2Me-Trp (or Mrp), 5-Methyl-DL-tryptophan, azatryptophan (7-azatryptophan), hydroxytryptophan (5-hydroxytryptophan), fluorotryptophan, aminotryptophan, tryptamine and desaminotryptophan, α-methyl-tryptophan; β-(3-benzothienyl)-D-alanine; β-(3-benzothienyl)-L-alanine; 1-methyl-tryptophan; 4-methyl-tryptophan; 5-benzyloxy-tryptophan; 5-bromo-tryptophan; 5-chloro-tryptophan; 5-fluoro-tryptophan; 5-hydroxy-tryptophan; 5-hydroxy-L-tryptophan; 5-methoxy-tryptophan; 5-methoxy-L-tryptophan; 5-methyl-tryptophan; 6-bromo-tryptophan; 6-chloro-d-tryptophan; 6-chloro-tryptophan; 6-fluoro-tryptophan; 6-methyl-tryptophan; 7-benzyloxy-tryptophan; 7-bromo-tryptophan; 7-methyl tryptophan; D-1,2,3,4-tetrahydro-norharman-3-carboxylic acid; 6-methoxy-1,2,3,4-tetrahydronorharman-1-carboxylic acid; L-1,2,3,4-tetrahydro-norharman-3-carboxylic acid; 5-methoxy-2-methyl-tryptophan; 2,3,4,9-tetrahydro-1H-β-carboline-3-carboxylic acid (Tca); and 6-chloro-L-tryptophan.

Analogs of alanine, glycine, valine, and leucine include β-alanine, aminoisobutyric acid (α or β), methylalanine, t-butylalanine, aminohexanoic acid, alpha,beta-diaminopropionic acid, propargylglycine, beta-cyclohexyl-L-alanine, beta-hydroxyleucine, aminocaproic acid, and allylglycine.

When a sequence comprises X/Z, it means that the sequence comprises amino acid X or amino acid Z.

As used herein, the terms “sequence identity” or “identity” refers to an exact amino acid to amino acid correspondence of two peptides. Percent of identity can be determined by a direct comparison of the sequence information between two molecules by aligning the sequences, counting the exact number of matches between the two aligned sequences, dividing by the length of the shorter sequence, and multiplying the result by 100.

The sequence identity can be determined by alignment of two peptide sequences using global or local alignment algorithms, depending on the length of the two sequences. Sequences of similar lengths are preferably aligned using global alignment algorithms (e.g., Needleman Wunsch) which aligns the sequences optimally over the entire length, while sequences of substantially different lengths are preferably aligned using a local alignment algorithm (e.g., Smith Waterman). Sequences may then be referred to as “substantially identical” or “essentially similar” when they (when optimally aligned by for example the programs GAP or BESTFIT using default parameters) share at least a certain minimal percentage of sequence identity. GAP uses the Needleman and Wunsch global alignment algorithm to align two sequences over their entire length (full length), maximizing the number of matches and minimizing the number of gaps. A global alignment is suitably used to determine sequence identity when the two sequences have similar lengths.

The term “retro form” or “retro analog” refers to a peptide comprising an amino acid sequence in reverse direction with respect to the reference peptide. The term “retroinverso form” or “retroinverso analog” refers to a peptide comprising an amino acid sequence in reverse direction with respect to the reference peptide and also have chirality of the amino acids inverted from L to D. The retro and retroinverso forms according to the present disclosure maintain the biological activity of the reference peptide, e.g., have the ability to inhibit the interaction between Neuropilin-1 and Plexin-A1.

The term “pharmaceutically acceptable salt” refers to salts of the peptides described herein that are pharmacologically acceptable and substantially non-toxic to the subject to which they are administered. More specifically, these salts retain the biological effectiveness and properties of the peptides and are formed from suitable non-toxic organic or inorganic acids or bases.

For example, these salts include acid addition salts of the peptides described herein which are sufficiently basic to form such salts. Such acid addition salts include acetates, adipates, alginates, lower alkanesulfonates such as a methanesulfonates, trifluoromethanesulfonates or ethanesulfonates, arylsulfonates such as a benzenesulfonates, 2-naphthalenesulfonates, or toluenesulfonates (also known as tosylates), ascorbates, aspartates, benzoates, benzenesulfonates, bisulfates, borates, butyrates, citrates, camphorates, camphorsulfonates, cinnamates, cyclopentanepropionates, digluconates, dodecylsulfates, ethanesulfonates, fumarates, glucoheptanoates, glycerophosphates, hemisulfates, heptanoates, hexanoates, hydrochlorides, hydrobromides, hydroiodides, hydrogen sulphates, 2-hydroxyethanesulfonates, itaconates, lactates, maleates, mandelates, methanesulfonates, nicotinates, nitrates, oxalates, pamoates, pectinates, perchlorates, persulfates, 3-phenylpropionates, phosphates, picrates, pivalates, propionates, salicylates, succinates, sulfates, sulfonates, tartrates, thiocyanates, undecanoates and the like.

Additionally, acids which are generally considered suitable for the formation of pharmaceutically useful salts from basic pharmaceutical compounds are discussed, for example, by P. Stahl et al., Camille G. (eds.) Handbook of Pharmaceutical Salts. Properties, Selection and Use. (2002) Zurich: Wiley-VCH; S. Berge et al., Journal of Pharmaceutical Sciences (1977) 66 (1) 1-19; P. Gould, International J. of Pharmaceutics (1986) 33 201-217; Anderson et al., The Practice of Medicinal Chemistry (1996), Academic Press, New York; and in The Orange Book (Food & Drug Administration, Washington, D.C.).

Also, where the peptides described herein are sufficiently acidic, the salts of the disclosure include base salts formed with an inorganic or organic base. Such salts include alkali metal salts such as sodium, lithium, and potassium salts; alkaline earth metal salts such as calcium and magnesium salts; metal salts such as aluminum salts, iron salts, zinc salts, copper salts, nickel salts and cobalt salts; inorganic amine salts such as ammonium or substituted ammonium salts, such as trimethylammonium salts; and salts with organic bases (for example, organic amines) such as chloroprocaine salts, dibenzylamine salts, dicyclohexylamine salts, diethanolamine salts, ethylamine salts (including diethylamine salts and triethylamine salts), ethylenediamine salts, glucosamine salts, guanidine salts, methylamine salts (including dimethylamine salts and trimethylamine salts), morpholine salts, N,N′-dibenzylethylenediamine salts, N-benzyl-phenethylamine salts, N-methylglucamine salts, phenylglycine alkyl ester salts, piperazine salts, piperidine salts, procaine salts, t-butyl amine salts, tetramethylammonium salts, t-octylamine salts, tris-(2-hydroxyethyl)amine salts, and tris(hydroxymethyl)aminomethane salts.

Such salts can be formed quite readily by those skilled in the art using standard techniques. Indeed, the chemical modification of a pharmaceutical peptide into a salt is a technique well known to pharmaceutical chemists, (See, e.g., H. Ansel et al., Pharmaceutical Dosage Forms and Drug Delivery Systems (6^{th} Ed. 1995) at pp. 196 and 1456-1457). Salts of the peptides described herein may be formed, for example, by reacting the peptide with an amount of acid or base, such as an equivalent amount, in a medium such as one in which the salt precipitates or in an aqueous medium followed by lyophilization.

By “increased”, “increase” or “enhance” is intended to refer to a measurement increased by at least 10, 20, 30, 40, 50, 60, 70, 80 or 90% when compared to the measurement measured in absence of the tested peptide in the same conditions. By “decreased” or “decrease” is intended to refer to a measurement decreased by at least 10, 20, 30, 40, 50, 60, 70, 80 or 90% when compared to the measurement measured in absence of the tested peptide in the same conditions.

As used herein, the term “treatment”, “treat” or “treating” refers to any act intended to ameliorate the health status of patients, such as cure, alleviate or delay of the disease or disorder. It includes preventive as well as therapeutic treatment.

As used herein, a “pharmaceutical composition” refers to a preparation of one or more of the active agents, such as comprising a peptide according to the disclosure, with optional other chemical components such as physiologically suitable carriers and excipients. The purpose of a pharmaceutical composition is to facilitate administration of the active agent to an organism. Compositions of the present disclosure can be in a form suitable for any conventional route of administration or use. In one embodiment, a “composition” typically intends a combination of the active agent, e.g., compound or composition, and a naturally-occurring or non-naturally-occurring carrier, inert (for example, a detectable agent or label) or active, such as an adjuvant, diluent, binder, stabilizer, buffers, salts, lipophilic solvents, preservative, adjuvant or the like and include pharmaceutically acceptable carriers. An “acceptable vehicle” or “acceptable carrier” as referred to herein, is any known compound or combination of compounds that are known to those skilled in the art to be useful in formulating pharmaceutical compositions.

“An effective amount” or a “therapeutic effective amount” as used herein refers to the amount of active agent required to confer therapeutic effect on the subject, either alone or in combination with one or more other active agents, e.g., the amount of active agent that is needed to treat the targeted disease or disorder, or to produce the desired effect. The “effective amount” will vary depending on the agent(s), the disease and its severity, the characteristics of the subject to be treated including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment.

As used herein, the term “medicament” refers to any substance or composition with curative or preventive properties against disorders or diseases.

The term “treatment” refers to any act intended to ameliorate the health status of patients such as therapy, prevention, prophylaxis and retardation of the disease or of the symptoms of the disease. It designates both a curative treatment and/or a prophylactic treatment of a disease. A curative treatment is defined as a treatment resulting in cure or a treatment alleviating, improving and/or eliminating, reducing and/or stabilizing a disease or the symptoms of a disease or the suffering that it causes directly or indirectly. A prophylactic treatment comprises both a treatment resulting in the prevention of a disease and a treatment reducing and/or delaying the progression and/or the incidence of a disease or the risk of its occurrence. In certain embodiments, such a term refers to the improvement or eradication of a disease, a disorder, an infection or symptoms associated with it. Treatments according to the present invention do not necessarily imply 100% or complete treatment. Rather, there are varying degrees of treatment of which one of ordinary skill in the art recognizes as having a potential benefit or therapeutic effect. Preferably, the term “treatment” refers to the application or administration of a composition including one or more active agents to a subject who has a disorder/disease.

As used herein, the terms “disorder” or “disease” refer to the incorrectly functioning organ, part, structure, or system of the body resulting from the effect of genetic or developmental errors, infection, poisons, nutritional deficiency or imbalance, toxicity, or unfavorable environmental factors. Preferably, these terms refer to a health disorder or disease, e.g., an illness that disrupts normal physical or mental functions.

As used herein, the terms “subject”, “individual” or “patient” are interchangeable and refer to an animal, preferably to a mammal, even more preferably to a human, including adult, child, newborn.

As used herein, the term “isolated” indicates that the recited material (e.g., compound, peptide, antibody, polypeptide, nucleic acid, etc.) is substantially separated from, or enriched relative to, other materials with which it occurs in nature. Particularly, an “isolated” peptide is one which has been identified and separated and/or recovered from a component of its natural environment.

### Antagonistic Peptides

The inventors defined the rule to design an antagonistic peptide of PlexinA1 as defined above.

In the peptides, the G/S-X-X-X-G motif or domain is involved in the activity. The possibility to replace the first Gly residue by a Ser residue is surprising and could not be predicted because the G-X-X-X-G motif has been generally considered essential. The X residues can be any amino acid. In a particular aspect, among the 3 X residues, no more than 2 residues can be a charged amino acid. Preferably, none of the 3 X residues are charged. In a particular aspect, they are selected among the aliphatic uncharged amino acids (Gly, Ala, Val, Leu and Ile) and more specifically can be selected in the group consisting of Gly, Val, Ile and Leu.

In addition, another important aspect for the design of the peptides is the anchorage of the G/S-X-X-X-G motif at the membrane and the distance between the membrane surface and the G/S-X-X-X-G motif. Indeed, the position of the motif is key, and improper positioning of the motif results in a reduction or loss of the antagonistic activity of the peptide.

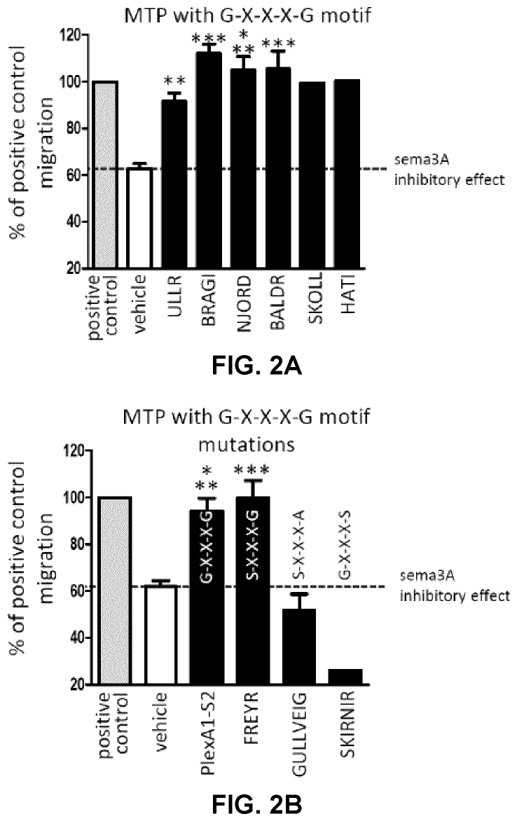

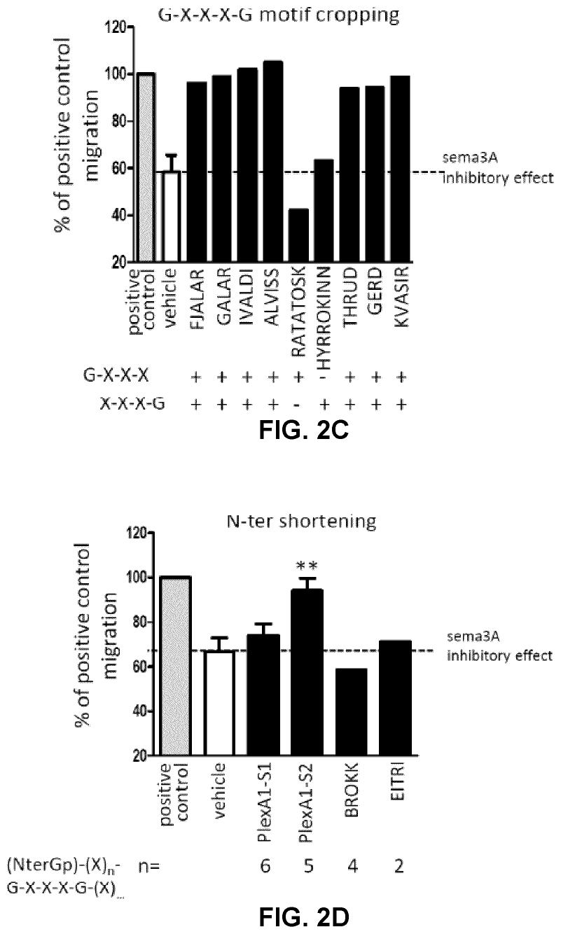

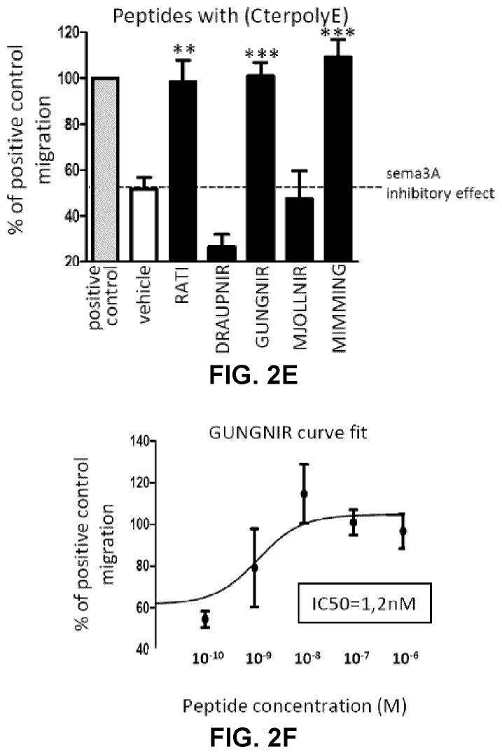

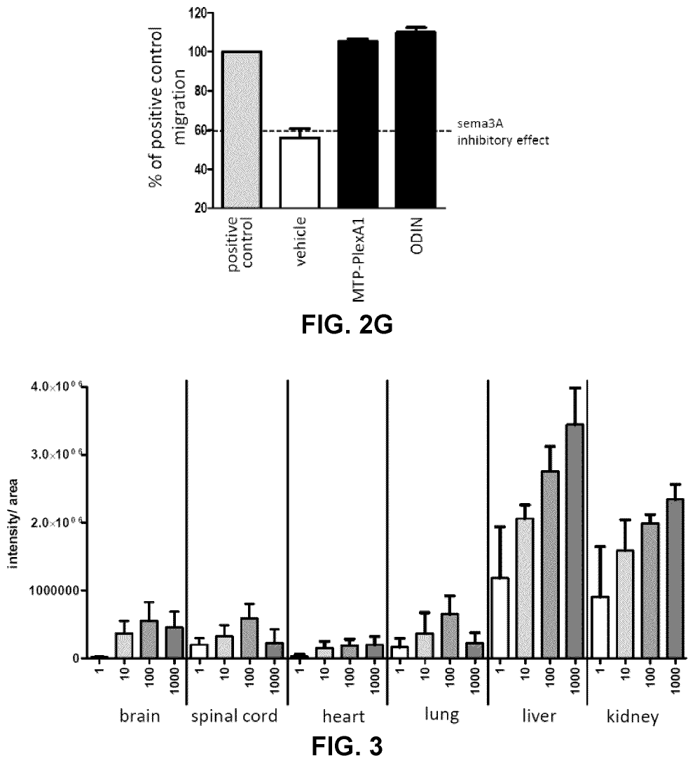

More specifically, if the peptide has a N-terminal membrane anchoring such as NterGp, five amino acids should preferably be inserted between the N-terminal membrane anchoring motif and the G/S-X-X-X-G motif. For instance, plexA1-S1 which has 6 amino acids, and BROKK or EITRI, which have respectively 4 amino acids and 2 amino acids, were not able to counteract Sema3A negative effect on migration of oligodendrocytes (FIG. 2D). On the opposite, peptides BALDR, FREYR, BRAGI, NJORD, ULLR, SKOLL, HATI, plexA1-S2, FJALAR, GALAR, IVALDI and ALVISS, all having 5 amino acids between the N-terminal membrane anchoring motif and the G/S-X-X-X-G motif, counteracted Sema3A negative effect on migration of oligodendrocytes (FIGS. 2A-2D).

If the peptide has a C-terminal membrane anchoring, then thirteen amino acids should preferably be inserted between the G/S-X-X-X-G motif and the C-terminal membrane anchoring motif with positively charged amino acids such as CterGp, and eleven amino acids should preferably be inserted between the G/S-X-X-X-G motif and the C-terminal membrane anchoring motif with negatively charged amino acids, such as CterpolyD/E. More particularly, in the context of a peptide having the C-terminal membrane anchoring motif with negatively charged amino acids, such as CterpolyD/E, the peptides KVASIR, GERD, THRUD, RATI, GUNGNIR and MIMMING having eleven amino acids between the G/S-X-X-X-G motif and the C-terminal membrane anchoring motif were all able to counteract Sema3A negative effect on migration of oligodendrocytes, in contrast to peptide DRAUPNIR which has twelve amino acids between the G/S-X-X-X-G motif and the C-terminal membrane anchoring motif.

Once these rules have been determined, the inventors surprisingly observed that the peptides can be shortened up to the motif G/S-X-X-X-G at the opposite side of the membrane anchoring motif. This aspect allows the design of shorter peptides.

For instance, compared to the peptide MTP-PlexA1 which is 29 amino acids in length, the peptides BALDR, FREYR, BRAGI, NJORD, ULLR and plexA1-S2 are only 18 amino acids in length, the peptide FJALAR only 17 amino acids in length, the peptide GALAR only 16 amino acids in length, the peptide IVALDI only 15 amino acids in length, and the peptides SKOLL, HATI and ALVISS only 14 amino acids in length. Similarly, including the 5 amino acids of the C-terminal polyE, peptides KVASIR, GERD, THRUD are respectively 26, 24 and 23 amino acids in length.

Accordingly, the present disclosure relates to a pharmaceutical composition comprising a peptide comprising, essentially consisting of or consisting of
a motif of the sequence G/S-X-X-X-G,
-(X)₁₁-(CterpolyD/E) directly linked at C-terminal end of the motif (i.e., G/S-X-X-X-G-(X)₁₁-(CterpolyD/E); (NterGp)-(X)₅- directly linked at N-terminal end of the motif (i.e., (NterGp)-(X)₅- G/S-X-X-X-G); or -(X)₁₃-(CterGp) directly linked at C-terminal end of the motif (i.e., G/S-X-X-X-G-(X)₁₃-(CterGp); and
with X being any amino acid but no more than 2 among the X being a charged amino acid;
with NterGp being a group of 3-5 amino acids including at least 2 charged amino acids, CterGp being a group of 3-5 amino acids including at least 3 positively charged amino acids, and CterpolyD/E being a group of 4-10 amino acids including at least 2 negatively charged amino acids,
   wherein the peptide inhibits the inhibitory effect of sema3A on migration and/or inhibits the interaction between Neuropilin-1/Plexin-A1 as measured by the method detailed in the specification; and
   wherein the peptide has not the sequence of MTP-PlexA1 (TLPAIVGIGGGGGLLLLVIVAVLIAYKRK, SEQ ID NO: 1).

The present disclosure also relates to a peptide comprising, essentially consisting of or consisting of
a first domain of the sequence G/S-X5-X6-X7-G, and
(i) a second domain of the formula I: -(X26-X27-X28-X29-X30-X31-X32-X33-X34-X35-X36)-(CterpolyD/E), directly linked at C-terminal end of the first domain (i.e., G/S-X-X-X-G-(X26-X27-X28-X29-X30-X31-X32-X33-X34-X35-X36)-(CterpolyD/E);
(ii) a second domain of the formula II: -(X13-X14-X15-X16-X17-X18-X19-X20-X21-X22-X23-X24-X25)-(CterGp), directly linked at C-terminal end of the first domain (i.e., G/S-X-X-X-G-(X13-X14-X15-X16-X17-X18-X19-X20-X21-X22-X23-X24-X25)-(CterGp); or
(iii) a second domain of the formula III: (NterGp)-(X8-X9-X10-X11-X12)-directly linked at N-terminal end of the first domain (i.e., (NterGp)-(X8-X9-X10-X11-X12)-G/S-X-X-X-G); with
   X5, X6 and X7 being any amino acids, preferably uncharged amino acids, and more preferably aliphatic uncharged amino acids such as Gly, Ala, Val, Leu and Ile;
   X8, X9, X10, X11 and X12 being any amino acid, preferably uncharged amino acids, and more preferably aliphatic or non-polar uncharged amino acids;
   X13, X14, X15, X16, X17, X18, X19, X20, X21, X22, X23, X24 and X25 being any amino acid, with no more than 2 amino acids among X13, X14, X15, X16, X17, X18, X19, X20, X21, X22, X23, X24 and X25 being a charged amino acid;
   X26, X27, X28, X29, X30, X31, X32, X33, X34, X35 and X36 being any amino acid, with no more than 2 amino acids among X26, X27, X28, X29, X30, X31, X32, X33, X34, X35 and X36 being a charged amino acid;
   NterGp is a membrane anchoring motif comprising charged amino acids, preferably a membrane anchoring motif comprising 3-5 amino acids including at least 2 charged amino acids,
   CterGp is a membrane anchoring motif comprising positively charged amino acids, preferably a membrane anchoring motif comprising 3-5 amino acids including at least 3 positively charged amino acids, and
   CterpolyD/E is a membrane anchoring motif comprising negatively charged amino acids, preferably a membrane anchoring motif comprising 4-10 amino acids including at least 2 negatively charged amino acids.

The peptide is associated to a functional feature of inhibiting the inhibitory effect of Sema3A on migration and/or of inhibiting the interaction between Neuropilin-1/Plexin-A1. Preferably, the peptide meets both features.

The interaction between Neuropilin-1/Plexin-A1 and the inhibition of this interaction by the peptide can be measured by any available method. More specifically, it can be measured by the Proximity ligation assay as specifically detailed in the Examples section. The interaction between

Neuropilin-1/Plexin-A1 is inhibited at least by 10, 20, 30, 40 or 50% in comparison to the interaction in absence of the peptide.

The effect of the peptide on the inhibitory effect of sema3A on migration can be measured by any available method. More specifically, it can be measured by Cell migration assay as detailed in the Example section. A peptide is considered as inhibiting the inhibitory effect of sema3A on migration if the peptide is able to restore at least 80% of the positive control migration.

The (NterGp), (CterGp) and (CterpolyD/E) are also called in the present disclosure a membrane anchoring motif. They are a group of amino acids allowing the anchorage of the peptide at the surface of the membrane. The membrane anchoring motif generally comprises one or several charged amino acids, for instance at least 2 charged amino acid, for example 2-7 charged amino acids.

More specifically, NterGp can be a group of 3-5 amino acids including at least 2 charged amino acids. For instance, NterGp can comprise one positively charged amino acid such as Glu or Asp and one negatively charged amino acid such as Lys or Arg; or two positively charged amino acid such as Glu or Asp; or two negatively charged amino acid such as Lys or Arg. In a very specific aspect, NterGp comprises or consists of KGD motif. Optionally, NterGp can further comprise an aromatic amino acid. In a specific aspect, NterGp has a sequence of 4 amino acids X1-X2-X3-X4 with X1 and X3 being two charged amino acids, preferably one positively charged and the other negatively charged, X2 being a small amino acid (e.g., G or A) and X4 being an aromatic amino acid (e.g., Y or W), more preferably NterGp being KGDW (SEQ ID NO: 116). The NterGp is preferably selected to be located at the extracellular side of the membrane.

In an embodiment, X5 is an aliphatic residue, such as L or I, preferably L. In an embodiment, X6 is P or I, preferably P. In an embodiment, X7 is an aliphatic residue, such as A or V, preferably A. In an embodiment, X8 is an aliphatic residue, such as L or I, preferably I. In an embodiment, X9 is an aliphatic and/or small residue, such as T or V, preferably V. In a further embodiment, X8-X9-X10-X11-X12 is LPAIT (SEQ ID NO:82), LPAIV (SEQ ID NO:83), IPALV (SEQ ID NO:84), LPALV (SEQ ID NO:85), LIAIV (SEQ ID NO:86) or LPVIV (SEQ ID NO:87).

Optionally, the peptide comprises, consists essentially of or consists of a sequence selected from:

(SEQ ID NO: 22)
(NterGp)-L/I-P/I-A/V-I/L-V/T-G/S-I/G/L-G/V-G-G;
(SEQ ID NO: 23)
(NterGp)-L/I-P/I-A/V-I/L-V/T-G/S-I/G/L-G/V-G-G-G/V;
(SEQ ID NO: 24)
(NterGp)-L/I-P/I-A/V-I/L-V/T-G/S-I/G/L-G/V-G-G-G/V-G/V;
(SEQ ID NO: 25)
(NterGp)-L/I-P/I-A/V-I/L-V/T-G/S-I/G/L-G/V-G-G-G/V-G/V-L/E;
(SEQ ID NO: 26)
(NterGp)-L/I-P/I-A/V-I/L-V/T-G/S-I/G/L-G/V-G-G-G/V-G/V-L/E-L;
(SEQ ID NO: 27)
(NterGp)-L/I-P/I-A/V-I/L-V/T-G/S-I/G/L-G/V-G-G-G/V-G/V-L/E-L-L;
(SEQ ID NO: 28)
(NterGp)-L/I-P/I-A/V-I/L-V/T-G/S-I/G/L-G/V-G-G-G/V-G/V-L/E-L-L-L/E;
(SEQ ID NO: 29)
(NterGp)-L/I-P/I-A/V-I/L-V/T-G/S-I/G/L-G/V-G-G-G/V-G/V-L/E-L-L-L/E-V;
(SEQ ID NO: 30)
(NterGp)-L/I-P/I-A/V-I/L-V/T-G/S-I/G/L-G/V-G-G-G/V-G/V-L/E-L-L-L/E-V-I;
(SEQ ID NO: 31)
(NterGp)-L/I-P/I-A/V-I/L-V/T-G/S-I/G/L-G/V-G-G-G/V-G/V-L/E-L-L-L/E-V-I-V;
(SEQ ID NO: 32)
(NterGp)-L/I-P/I-A/V-I/L-V/T-G/S-I/G/L-G/V-G-G-G/V-G/V-L/E-L-L-L/E-V-I-V-A/E;
(SEQ ID NO: 33)
(NterGp)-L/I-P/I-A/V-I/L-V/T-G/S-I/G/L-G/V-G-G-G/V-G/V-L/E-L-L-L/E-V-I-V-A/E-V;
(SEQ ID NO: 34)
(NterGp)-L/I-P/I-A/V-I/L-V/T-G/S-I/G/L-G/V-G-G-G/V-G/V-L/E-L-L-L/E-V-I-V-A/E-V-L;
and
(SEQ ID NO: 35)
(NterGp)-L/I-P/I-A/V-I/L-V/T-G/S-I/G/L-G/V-G-G-G/V-G/V-L/E-L-L-L/E-V-I-V-A/E-V-L-I;

wherein NterGp is as defined above, e.g., a group of 3-5 amino acids including at least 2 charged amino acids, and
wherein the sequence may comprise 1, 2 or 3 substitutions of one amino acid (e.g., conservative substitutions) at any position except the bold residues and/or the addition of 1 to 6 amino acids at the N-terminal and/or C-terminal end.

In an embodiment, the peptide comprises a second domain of the formula II, and wherein the peptide comprises no more than 5 amino acids at the N-terminal end of the first domain. In an embodiment, the peptide comprises 4 amino acid or less at the N-terminal end of the first domain. In an embodiment, the peptide comprises 3 amino acid or less at the N-terminal end of the first domain. In an embodiment, the peptide comprises 2 amino acid or less at the N-terminal end of the first domain. In an embodiment, the peptide comprises 1 amino acid at the N-terminal end of the first domain. In an embodiment, the peptide does not comprise any amino acid at the N-terminal end of the first domain.

In an embodiment, the peptide comprises a second domain of the formula II, and wherein the peptide has a length of 28 amino acids or less, 27 amino acids or less, or 26 amino acids or less. In an embodiment, the peptide comprises a second domain of the formula II and has a length of 23 to 26 amino acids.

In an embodiment, X13 is an aliphatic and/or small residue, such as G or V, preferably G. In an embodiment, X14 is an aliphatic and/or small residue, such as G or V, preferably G. In an embodiment, X15 is L or E, preferably L. In an embodiment, X16 is an aliphatic residue, preferably L. In an embodiment, X17 is an aliphatic residue, preferably L. In an embodiment, X18 is L or E, preferably L. In an embodiment, X19 is an aliphatic residue, preferably V. In an embodiment, X20 is an aliphatic residue, preferably I. In an embodiment, X21 is an aliphatic residue, preferably V. In an embodiment, X22 is an A or E, preferably A. In an embodiment, X23 is an aliphatic residue, preferably V. In an embodiment, X24 is an aliphatic residue, preferably L. In an embodiment, X25 is an aliphatic residue, preferably L.

In a further embodiment, X13-X14-X15-X16-X17-X18-X19-X20-X21-X22-X23-X24-X25 is GGLLLLVIVAVLI (SEQ ID NO:88).

More specifically, CterGp can be a 3-5 amino acids including at least 3 positively charged amino acids. For instance, CterGp can include KRK, KKR, RRK or KRR. Optionally, it can further comprise an aromatic amino acid and/or a small amino acid. In a very specific aspect, CterGp has a sequence of 5 amino acids X′1-X′2-X′3-X′4-X′5 with X′1 being a small amino acid (e.g., G or A), X′2 being an aromatic amino acid (e.g., Y or W), X′3, X′4 and X′5 being a basic amino acid, more preferably CterGp being a sequence selected from AYKRK (SEQ ID NO: 76), AYKKR (SEQ ID NO: 77), AYKRR (SEQ ID NO: 78), AYRRK (SEQ ID NO: 79) and AYRKK (SEQ ID NO: 80). The CterGp is preferably selected to be located at the intracellular side of the membrane.

Optionally, the peptide comprises, consists essentially of, or consists of a sequence selected from

(SEQ ID NO: 36)
A/V-I/L-V/T-G/S-I/G/L-G/V-G-G-G/V-G/V-L/E-L-L-L/E-V-I-V-A/E-V-L-I-(CterGp);
(SEQ ID NO: 37)
I/L-V/T-G/S-I/G/L-G/V-G-G-G/V-G/V-L/E-L-L-L/E-V-I-V-A/E-V-L-I-(CterGp);
(SEQ ID NO: 38)
V/T-G/S-I/G/L-G/V-G-G-G/V-G/V-L/E-L-L-L/E-V-I-V-A/E-V-L-I-(CterGp);
and
(SEQ ID NO: 39)
G/S-I/G/L-G/V-G-G-G/V-G/V-L/E-L-L-L/E-V-I-V-A/E-V-L-I-(CterGp);

wherein CterGp is as defined above, e.g., a group of 3-5 amino acids including at least 3 positively charged amino acids,
wherein the sequence may comprise 1, 2 or 3 substitutions of one amino acid (e.g., conservative substitutions) at any position except the bold residue.

More specifically, CterpolyD/E includes a cluster of negatively charged amino acids, especially Glu. For instance, it can be a group of 4-10 amino acids including at least 2, at least 3 or at least 4 negatively charged amino acids, e.g., Glu, Asp or a combination of Glu and Asp. The CterpolyD/E domain may include 2, 3, 4 or 5 Glu residues, 2, 3, 4 or 5 Asp residues, or a combination of 2, 3, 4 or 5 Glu and Asp residues. In a specific aspect, CterpolyD/E has a sequence of -X″1-X″2-(Z)ₙ with X″1 being a small amino acid (e.g., G or A), X″2 being an aromatic amino acid (e.g., Y or W), Z being D or E, and n being an integer between 2-10, preferably 4-6; or a sequence of -X″1-X″1bis-X″2-(Z), with X″1 and X″1bis being long aliphatic amino acids such as I or L, X″2 being an aromatic amino acid (e.g., Y or W), Z being D or E, and n being an integer between 2-10, preferably 4-6. In a very specific aspect, CterpolyD/E has a sequence of -X″1-X″2-(E)ₙ with X″1 being a small amino acid (e.g., G or A), X″2 being an aromatic amino acid (e.g., Y or W), and n being an integer between 2-10, preferably 4-6; or a sequence of -X″1-X″1bis-X″2-(E)ₙ with X″1 and X″1bis being long aliphatic amino acids such as I or L, X″2 being an aromatic amino acid (e.g., Y or W), and n being an integer between 2-10, preferably 4-6. The CterpolyD/E is preferably selected to be located at the intracellular side of the membrane. In an embodiment, CterpolyD/E comprises or consists of the sequence AYEEEEE (SEQ ID NO:89) or LIYEEEEE (SEQ ID NO:90).

The presence of a CterpolyD/E membrane anchoring motif is a particularly preferred aspect because the peptides comprising such a membrane anchoring motif are soluble and stable, allowing a plasmatic half-life longer than 24 h and a biodistribution suitable for reaching target organs such as brain and spinal cord.

In an embodiment, X26 is an aliphatic and/or small residue, such as G or V, preferably V. In an embodiment, X27 is an aliphatic and/or small residue, such as G or V, preferably G. In an embodiment, X28 is L or E, preferably L. In an embodiment, X29 is an aliphatic residue, preferably L. In an embodiment, X30 is an aliphatic residue, preferably L. In an embodiment, X31 is L or E, preferably L. In an embodiment, X32 is an aliphatic residue, preferably V. In an embodiment, X33 is an aliphatic residue, preferably I. In an embodiment, X34 is an aliphatic residue, preferably V. In an embodiment, X35 is an A or E, preferably E. In an embodiment, X36 is an aliphatic residue, preferably V.

In an embodiment, X26-X27-X28-X29-X30-X31-X32-X33-X34-X35-X36 is VGLLLEVIVEV (SEQ ID NO: 91), or a variant thereof having 1, 2 or 3 amino acid substitutions, such as conservative substitutions, except at the residues underlined. In an embodiment, X26-X27-X28-X29-X30-X31-X32-X33-X34-X35-X36 is VGLLLEVIVEV (SEQ ID NO: 91), or a variant thereof having 1, 2 or 3 amino acid substitutions, such as conservative substitutions, except at the residues underlined. In an embodiment, the variant has 1 or 2 amino acid substitutions, such as conservative substitutions. In an embodiment, the variant has 1 amino acid substitution, such as a conservative substitution.

In an embodiment, X26-X27-X28-X29-X30-X31-X32-X33-X34-X35-X36 is VGLLLEVIVEV (SEQ ID NO: 91), GGELLLVIVE (SEQ ID NO: 92), VVLLLEVIVEV (SEQ ID NO: 93), VGLLVEVIVEV (SEQ ID NO:117), VGLVLEVIVEV (SEQ ID NO:118).

In another embodiment, the peptide comprises, consists essentially of or consists of a sequence selected from:

(SEQ ID NO: 2)
G/S-I/G/L-G/V-G-G-G/V-G/V-L/E-L-L-L/E-V-I-V-A/E-V-(CterpolyD/E);
(SEQ ID NO: 3)
V/T-G/S-I/G/L-G/V-G-G-G/V-G/V-L/E-L-L-L/E-V-I-V-A/E-V-(CterpolyD/E);
(SEQ ID NO: 4)
I/L-V/T-G/S-I/G/L-G/V-G-G-G/V-G/V-L/E-L-L-L/E-V-I-V-A/E-V-(CterpolyD/E);
(SEQ ID NO: 5)
A/V-I/L-V/T-G/S-I/G/L-G/V-G-G-G/V-G/V-L/E-L-L-L/E-V-I-V-A/E-V-(CterpolyD/E);
(SEQ ID NO: 6)
P/I-A/V-I/L-V/T-G/S-I/G/L-G/V-G-G-G/V-G/V-L/E-L-L-L/E-V-I-V-A/E-V-(CterpolyD/E);
(SEQ ID NO: 7)
I/L-P/I-A/V-I/L-V/T-G/S-I/G/L-G/V-G-G-G/V-G/V-L/E-L-L-L/E-V-I-V-A/E-V-(CterpolyD/E);
and
(SEQ ID NO: 8)
T-I/L-P/I-A/V-I/L-V/T-G/S-I/G/L-G/V-G-G-G/V-G/V-L/E-L-L-L/E-V-I-V-A/E-V-(CterpolyD/E);

wherein CterpolyD/E is as defined above, e.g., a group of 4-10 amino acids including at least 2 negatively charged amino acids, and
wherein the sequence may comprise 1, 2 or 3 substitutions of one amino acid (e.g., conservative substitutions) at any position except the bold residues and/or the addition of 1 to 6 amino acids at the N-terminal or C-terminal end.

Optionally, CterpolyD/E is according to any particular aspect or embodiment disclosed herein.

Optionally, the peptide comprises, consists essentially of or consists of the following amino acid sequence

(SEQ ID NO: 9)
G/S-I/G/L-G/V-G-G-G/V-G/V-L/E-L-L-L/E-V-I-V-E-V-A/LI-Y-(E)ₙ;
or
(SEQ ID NO: 119)
G/S-L-V-G-G-G/V-G/V-L/E-L-L/V-L/E-V-I-V-E-V-A-Y-(E/D)ₙ;

wherein the sequence may further comprise 1, 2 or 3 substitutions of one amino acid (e.g., conservative substitutions) at any position except the bold residues and/or the addition of 1 to 6 amino acids at the N-terminal or C-terminal end, and wherein n is an integer between 2-10, preferably 4-6. “A/LI” means that the peptide comprises either an amino acid A or two amino acids LI. Optionally, n is an integer selected from the group consisting of 2, 3, 4, 5, 6, 7, 8, 9 and 10, preferably of 4, 5, 6, 7, and 8, for instance 4, 5 or 6. Optionally, the peptide may comprise addition of 1 to 6 amino acids at the N-terminal end.

Optionally, the peptide comprises, consists essentially of or consists of a sequence selected from:

(SEQ ID NO: 10)
AIT-G/S-LVGGVGLLLEVIVEVAYEEEEE;
(SEQ ID NO: 11)
T-G/S-LVGGVGLLLEVIVEVAYEEEEE;
(SEQ ID NO: 12)
G/S-LVGGVGLLLEVIVEVAYEEEEE;
(SEQ ID NO: 13)
TLPAIV-G/S-IGGGGGELLLVIVEVLIYEEEEE;
(SEQ ID NO: 14)
TLPAIT-G/S-LVGGVGLLLEVIVEVAYEEEEE;
and
(SEQ ID NO: 15)
TLPAIV-G/S-IGGGVVLLLEVIVEVAYEEEEE;

wherein the sequence may further comprise 1, 2 or 3 substitutions of one amino acid (e.g., conservative substitutions) at any position except the bold residues and the addition of 1 to 6 amino acids at the N-terminal or C-terminal end.

In a very specific aspect, the peptide consists of a sequence selected from

(SEQ ID NO: 10)
AIT-G/S-LVGGVGLLLEVIVEVAYEEEEE;
(SEQ ID NO: 11)
T-G/S-LVGGVGLLLEVIVEVAYEEEEE;
(SEQ ID NO: 12)
G/S-LVGGVGLLLEVIVEVAYEEEEE;
(SEQ ID NO: 13)
TLPAIV-G/S-IGGGGGELLLVIVEVLIYEEEEE;
(SEQ ID NO: 14)
TLPAIT-G/S-LVGGVGLLLEVIVEVAYEEEEE;
and
(SEQ ID NO: 15)
TLPAIV-G/S-IGGGVVLLLEVIVEVAYEEEEE;

wherein the sequence may further comprise 1, 2 or 3 substitutions of one amino acid (e.g., conservative substitutions) at any position except the bold residues.

In an additional very specific aspect, the peptide consists of a sequence selected from:

(SEQ ID NO: 10)
AIT-G/S-LVGGVGLLLEVIVEVAYEEEEE;
(SEQ ID NO: 11)
T-G/S-LVGGVGLLLEVIVEVAYEEEEE;
(SEQ ID NO: 12)
G/S-LVGGVGLLLEVIVEVAYEEEEE;
(SEQ ID NO: 13)
TLPAIV-G/S-IGGGGGELLLVIVEVLIYEEEEE;
(SEQ ID NO: 14)
TLPAIT-G/S-LVGGVGLLLEVIVEVAYEEEEE;
and
(SEQ ID NO: 15)
TLPAIV-G/S-IGGGVVLLLEVIVEVAYEEEEE.

Optionally, the peptide comprises, consists essentially of or consists of a sequence selected from:

(SEQ ID NO: 16)
AITGLVGGVGLLLEVIVEVAYEEEEE;
(SEQ ID NO: 17)
TGLVGGVGLLLEVIVEVAYEEEEE;
(SEQ ID NO: 18)
GLVGGVGLLLEVIVEVAYEEEEE;
(SEQ ID NO: 19)
TLPAIVGIGGGGGELLLVIVEVLIYEEEEE;
(SEQ ID NO: 20)
TLPAITGLVGGVGLLLEVIVEVAYEEEEE;
and
(SEQ ID NO: 21)
TLPAIVSIGGGVVLLLEVIVEVAYEEEEE;

wherein the sequence may further comprise 1, 2 or 3 substitutions of one amino acid (e.g., conservative substitutions) at any position except the bold residues and the addition of 1 to 6 amino acids at the N-terminal or C-terminal end.

In a very specific aspect, the peptide consists of a sequence selected from

(SEQ ID NO: 16)
AITGLVGGVGLLLEVIVEVAYEEEEE;
(SEQ ID NO: 17)
TGLVGGVGLLLEVIVEVAYEEEEE;
(SEQ ID NO: 18)
GLVGGVGLLLEVIVEVAYEEEEE;
(SEQ ID NO: 19)
TLPAIVGIGGGGGELLLVIVEVLIYEEEEE;
(SEQ ID NO: 20)
TLPAITGLVGGVGLLLEVIVEVAYEEEEE;
and
(SEQ ID NO: 21)
TLPAIVSIGGGVVLLLEVIVEVAYEEEEE;

wherein the sequence may further comprise 1, 2 or 3 substitutions of one amino acid (e.g., conservative substitutions) at any position except the bold residues.

In an additional very specific aspect, the peptide consists of a sequence selected from

(SEQ ID NO: 16)
AITGLVGGVGLLLEVIVEVAYEEEEE;
(SEQ ID NO: 17)
TGLVGGVGLLLEVIVEVAYEEEEE;
(SEQ ID NO: 18)
GLVGGVGLLLEVIVEVAYEEEEE;
(SEQ ID NO: 19)
TLPAIVGIGGGGGELLLVIVEVLIYEEEEE;
(SEQ ID NO: 20)
TLPAITGLVGGVGLLLEVIVEVAYEEEEE;
and
(SEQ ID NO: 21)
TLPAIVSIGGGVVLLLEVIVEVAYEEEEE.

Accordingly, the peptide of the present disclosure may have an amino acid sequence selected from:

(SEQ ID NO: 40)
KGDWLPAIT-G/S-LVGGVGLL
(SEQ ID NO: 41)
KGDWLPAIV-G/S-IGGGVVLL
(SEQ ID NO: 42)
KGDWIPALV-G/S-GGGGGGLL
(SEQ ID NO: 43)
KGDWLPALV-G/S-IGGGVGLL
(SEQ ID NO: 44)
KGDWIPALV-G/S-LGGGGGLL
(SEQ ID NO: 45)
KGDWLIAIV-G/S-IGGG
(SEQ ID NO: 46)
KGDWLPVIV-G/S-IGGG
(SEQ ID NO: 47)
KGDWLPAIV-G/S-IGGGGGLL
(SEQ ID NO: 48)
KGDWLPAIV-G/S-IGGGGGL
(SEQ ID NO: 49)
KGDWLPAIV-G/S-IGGGGG
(SEQ ID NO: 50)
KGDWLPAIV-G/S-IGGGG
(SEQ ID NO: 51)
KGDWLPAIV-G/S-IGGG
(SEQ ID NO: 10)
AIT-G/S-LVGGVGLLLEVIVEVAYEEEEE
(SEQ ID NO: 11)
T-G/S-LVGGVGLLLEVIVEVAYEEEEE
(SEQ ID NO: 12)
G/S-LVGGVGLLLEVIVEVAYEEEEE
(SEQ ID NO: 13)
TLPAIV-G/S-IGGGGGELLLVIVEVLIYEEEEE
(SEQ ID NO: 14)
TLPAIT-G/S-LVGGVGLLLEVIVEVAYEEEEE
(SEQ ID NO: 15)
TLPAIV-G/S-IGGGVVLLLEVIVEVAYEEEEE

wherein the sequence may comprise 1, 2 or 3 substitutions of one amino acid (e.g., conservative substitutions) at any position except the bold residue and the addition of 1 to 6 amino acids at the N-terminal or C-terminal end.

In a very particular aspect, the peptide has an amino acid sequence selected in the group consisting of

(SEQ ID NO: 52)
KGDWLPAITGLVGGVGLL
(SEQ ID NO: 53)
KGDWLPAIVSIGGGVVLL
(SEQ ID NO: 54)
KGDWIPALVGGGGGGGLL
(SEQ ID NO: 55)
KGDWLPALVSIGGGVGLL
(SEQ ID NO: 56)
KGDWIPALVGLGGGGGLL
(SEQ ID NO: 57)
KGDWLIAIVGIGGG
(SEQ ID NO: 58)
KGDWLPVIVGIGGG
(SEQ ID NO: 59)
KGDWLPAIVGIGGGGGLL
(SEQ ID NO: 60)
KGDWLPAIVGIGGGGGL
(SEQ ID NO: 61)
KGDWLPAIVGIGGGGG
(SEQ ID NO: 62)
KGDWLPAIVGIGGGG
(SEQ ID NO: 63)
KGDWLPAIVGIGGG
(SEQ ID NO: 16)
AITGLVGGVGLLLEVIVEVAYEEEEE
(SEQ ID NO: 17)
TGLVGGVGLLLEVIVEVAYEEEEE
(SEQ ID NO: 18)
GLVGGVGLLLEVIVEVAYEEEEE
(SEQ ID NO: 19)
TLPAIVGIGGGGGELLLVIVEVLIYEEEEE
(SEQ ID NO: 20)
TLPAITGLVGGVGLLLEVIVEVAYEEEEE
(SEQ ID NO: 21)
TLPAIVSIGGGVVLLLEVIVEVAYEEEEE

wherein the sequence may comprise 1, 2 or 3 substitutions of one amino acid (e.g., conservative substitutions) at any position except the bold residues and the addition of 1 to 6 amino acids at the N-terminal or C-terminal end.

In a particular aspect, the peptide has a length of no more than 35, 34, 33, 32, 31 or 30 amino acids, in particular of no more than 25 or 20 amino acids.

In a particular aspect, the peptide is not found in nature. The peptide is a non-natural peptide. It can be purified, isolated or recombinant. It can be produced by well-known peptide synthesis methods.

The N- and C-termini of the peptides described herein may be optionally protected against proteolysis. In a preferred embodiment, the N-terminus may be in the form of an acetyl group, and/or the C-terminus may be in the form of an amide group. In a preferred embodiment, the peptide has a free C-terminal end.

Alternatively or in addition, internal modifications of the peptides to be resistant to proteolysis are also envisioned, e.g. wherein at least a —CONH— peptide bond is modified and replaced by a (CH₂NH) reduced bond, a (NHCO) retro-inverso bond, a (CH₂—O) methylene-oxy bond, a (CH₂—S) thiomethylene bond, a (CH₂CH₂) carba bond, a (CO—CH₂) cetomethylene bond, a (CHOH—CH₂) hydroxyethylene bond), a (N—N) bound, a E-alcene bond or also a —CH═CH-bond. In a particular aspect, the peptide can be a retro analog of any peptide disclosed herein (the same sequence but in the reverse direction) or a retroinverso analog of any peptide disclosed herein (the same sequence but in the reverse direction and a chirality of amino acid inverted from L to D).

For instance, the peptide may be modified by acetylation, acylation, amidation, cross-linking, cyclization, disulfide bond formation, formation of covalent cross-links, formation of cysteine, formation of pyroglutamate, formylation, gamma-carboxylation, glycosylation, GPI anchor formation, hydroxylation, iodination, methylation, myristylation, oxidation, phosphorylation, and the like.

The peptide according to the disclosure may comprise one or more amino acids which are rare amino acids in particular hydroxyproline, hydroxylysine, allohydroxylysine, 6-N-methylysine, N-ethylglycine, N-methylglycine, N-ethylasparagine, allo-isoleucine, N-methylisoleucine, N-methylvaline, pyroglutamine, aminobutyric acid; or synthetic amino acids in particular ornithine, norleucine, norvaline and cyclohexyl-alanine.

Optionally, the peptide can be linked to additional moiety, optionally through a linker or spacer (e.g., diglycine), e.g., to form a conjugate. Optionally, the peptide can be part of a fusion protein. The additional moiety can be a homing peptide; a stabilizing agent such as PEG (polyethylene glycol), oligo-N-methoxy-ethylglycine (NMEG), albumin, an albumin-binding protein or an immunoglobulin Fc domain; an affinity tag such as an immune-tag, biotin, lectin, or chelator; a purification tag such as a His-tag; a detectable label such as an optical tag, a chelated lanthamide, a fluorescent dye, or a FRET/BRET acceptor/donor; a targeting moiety; a secretion signal peptide; or a combination thereof. This additional moiety can for example allow a specific targeting of cells, for instance cancer cells or oligodendrocytes. For instance, peptides can be combined with targeting moieties attached to nanocarriers as detailed in Nguyen et al. (J Control Release. (2019) 298:142-153) or in Gamper et al. (2019, Cancers, 11, 1609). Accordingly, in a particular aspect, the present disclosure relates to a nanocarrier (e.g., nanoparticles) linked to the peptide of the present disclosure. The nanocarrier can be for instance an artificial nanocarrier or a virus-derived nanoparticle (Steinmetz et al., Org. Biomol. Chem, 2007, 5, 2891-2902; Hashizume et al., Am. J. Pathol. 2000, 156, 1363-1380; Maeda et al, J. Control. Release, 2000, 65, 271-284; Allen et al., Science 2004, 303, 1818-1822; Cho et al., J. Vis. Exp. 2011, 52, e2808; Gamper et al, 2019, Cancers, 11, 1609). The peptide may be linked to a moiety to target the peptide to the nervous system (e.g., central nervous system (CNS)) and/or to facilitate entry of the peptide into the CNS across the blood-brain barrier.

The additional moiety can be added either at the N-terminal end or C-terminal end of the peptide, or may be attached to the side chain of one or more of the amino acids of the peptide (e.g., to lysine or cysteine residues). Preferably, the additional moiety is fused or conjugated at the end carrying the membrane anchoring motif.

In another aspect of the disclosure, peptides are covalently bound to a polymer such as polyethylene glycol (PEG) molecule by their C-terminal terminus or a lysine residue, notably a PEG of 1500 or 4000 MW, for a decrease in urinary clearance and in therapeutic doses used and for an increase of the half-life in blood plasma. In yet another embodiment, peptide half-life is increased by including the peptide in a biodegradable and biocompatible polymer material for drug delivery system forming microspheres. Polymers and copolymers are, for instance, poly(D,L-lactide-co-glycolide) (PLGA) (as illustrated in US2007/0184015).

The disclosure also encompasses pharmaceutically acceptable salts of a peptide according to the disclosure. Pharmaceutically acceptable salts may, for example, be salts of pharmaceutically acceptable mineral acids such as hydrochloric acid, hydrobromic acid, sulphuric acid and phosphoric acid; salts of pharmaceutically acceptable organic acids such as acetic acid, citric acid, maleic acid, malic acid, succinic acid, ascorbic acid and tartaric acid; salts of pharmaceutically acceptable mineral bases such as salts of sodium, potassium, calcium, magnesium or ammonium; or salts of organic bases which contain a salifiable nitrogen, commonly used in pharmaceutical technique. The methods for preparing said salts are well known to one of skill in the art.

In a particular aspect, the present disclosure relates to a nucleic acid, such as an mRNA molecule, encoding a peptide according to the present disclosure.

### Pharmaceutical Composition

The present disclosure relates to a pharmaceutical composition comprising a peptide as disclosed herein. The peptide is the active ingredient.

The pharmaceutical composition may further comprise a pharmaceutically acceptable vehicle.

The pharmaceutical composition comprising the peptide is formulated in accordance with standard pharmaceutical practice (Lippincott Williams & Wilkins, 2000 and Encyclopedia of Pharmaceutical Technology, eds. J. Swarbrick and J. C. Boylan, 1988-1999, Marcel Dekker, New York) known by a person skilled in the art.

For instance, the composition can comprise emulsions, microemulsions, oil-in-water emulsions, anhydrous lipids and other types of emulsions. The composition may further comprise one or more additives such as diluents, excipients, stabilizers and preservatives.

In one aspect, the present disclosure provides a stable formulation for parenteral injection of the pharmaceutical composition according to the present disclosure comprising a peptide or a salt thereof, wherein the peptide has been dried and then is reconstituted in a solvent prior to use. The peptide (or, in embodiments where the formulation comprises two or more peptides, each of the peptides) is mixed with a non-volatile buffer and dried to a dry peptide powder. Suitable buffers include, but are not limited to, glycine buffers, citrate buffers, phosphate buffers, and mixtures thereof. In one aspect, the buffer is a glycine buffer. In another aspect, the buffer is a mixture of citrate buffer and phosphate buffer. Alternatively, the pharmaceutical composition according to the present disclosure may be stored in an aqueous state. The solution may contain, if desired, further additives or excipients, which must be compatible with the active principle and, if they are not removed during the freeze-drying stage, they must also be compatible with the route of administration.

For oral administration, the composition can be formulated into conventional oral dosage forms such as tablets, capsules, powders, granules and liquid preparations such as syrups, elixirs, and concentrated drops. Non-toxic solid carriers or diluents may be used which include, for example, pharmaceutical grades of mannitol, lactose, starch, magnesium stearate, sodium saccharine, talcum, cellulose, glucose, sucrose, magnesium, carbonate, and the like. For compressed tablets, binders, which are agents which impart cohesive qualities to powdered materials, are also necessary. For example, starch, gelatine, sugars such as lactose or dextrose, and natural or synthetic gums can be used as binders. Disintegrants are also necessary in the tablets to facilitate break-up of the tablet. Disintegrants include starches, clays, celluloses, algins, gums and crosslinked polymers. Moreover, lubricants and glidants are also included in the tablets to prevent adhesion to the tablet material to surfaces in the manufacturing process and to improve the flow characteristics of the powder material during manufacture. Colloidal silicon dioxide is most commonly used as a glidant and compounds such as talc or stearic acids are most commonly used as lubricants.

For transdermal administration, the composition can be formulated into ointment, cream or gel form and appropriate penetrants or detergents could be used to facilitate permeation, such as dimethyl sulfoxide, dimethyl acetamide and dimethylformamide.

For transmucosal administration, nasal sprays, intrapulmonary inhalation, rectal or vaginal suppositories can be used. In one embodiment, the peptide, salt thereof or composition of the present disclosure may be administered by the intrapulmonary route using either a dry powder or liquid formulation administered using an intrapulmonary drug delivery device according to methods known in the art. The active compound (peptide or salt thereof) can be incorporated into any of the known suppository bases by methods known in the art. Examples of such bases include cocoa butter, polyethylene glycols (carbowaxes), polyethylene sorbitan monostearate, and mixtures of these with other compatible materials to modify the melting point or dissolution rate.

The form of the pharmaceutical compositions, the route of administration, the dosage and the regimen naturally depend upon the condition to be treated, the severity of the illness, the age, weight, and sex of the patient, etc.

The pharmaceutical or therapeutic compositions of the present disclosure can be formulated for a topical, oral, parenteral, intranasal, intravenous, intramuscular, intratumoral, subcutaneous or intraocular administration and the like. For parenteral administration, the composition may be injected intradermally, subcutaneously, intramuscularly, or intravenously.

In an embodiment, the pharmaceutical or therapeutic compositions of the present disclosure is formulated for administration into the nervous system, for example the central nervous system (CNS) of a subject, such as intracranial injection or injection into the cerebrospinal fluid (e.g., intrathecal injection).

In a particular aspect, the pharmaceutical composition according to the present disclosure comprises between 0.01 ng and 10 mg of the peptide of the present disclosure by kg of body weight. In one aspect, pharmaceutical composition according to the present disclosure comprises between 0.1 ng and 1 g of the peptide of the present disclosure by kg of body weight.

### Therapeutic Uses

The antagonistic peptide of the present disclosure can be useful for the treatment of any disease or disorder that can be prevented or treated by blocking the Plexin-A1 receptor involved in the inhibitory signaling pathway Sema3A-neuropilin 1-plexin-A1.

In an embodiment, the present disclosure relates to the peptide, salt thereof or composition as described herein for use in the treatment of a demyelinating disease, to the use of the peptide, salt thereof or composition as described herein for the manufacture of a medicament for the treatment of a demyelinating disease, and to a method for the treatment of a myelinating disease in a subject, comprising administering a therapeutically efficient amount of the peptide, salt thereof or composition as described herein the subject. The therapeutic effect of the peptide or salt thereof may involve the inhibition of the Sema3A inhibitory effect on oligodendrocyte migration and differentiation, thereby reducing the demyelination and/or promoting remyelination.

The demyelinating disease can be an autoimmune demyelinating disease. In an embodiment, the demyelinating disease is multiple sclerosis, transverse myelitis, neuromyelitis optica (Devic's disease), acute hemorrhagic leukoencephalitis, acute disseminated encephalomyelitis (ADEM), diffuse cerebral sclerosis of Schilder, adrenoleukodystrophy, Alexander disease, Canavan disease, Balo's disease, Charcot-Marie-Tooth disease (CMT), HTLV-I Associated Myelopathy (HAM), globoid cell leukodystrophy, metachromatic leukodystrophy, Pelizaeus-Merzbacher disease, progressive multifocal leukoencephalopathy, Marchiafava-Bignami disease, central pontine myelinolysis, and polyradiculoneuropathy including Guillain-Barre syndrome (GBS) or chronic inflammatory demyelinating polyradiculoneuropathy. In a further embodiment, the demyelinating disease is an autoimmune or inflammatory demyelinating disease, such as multiple sclerosis. In a further embodiment, the multiple sclerosis is recurrent-remitting multiple sclerosis. In another embodiment, the multiple sclerosis is progressive multiple sclerosis.

In addition, the peptide, salt thereof or composition can be used in combination with other active ingredients used for the treatment of a demyelinating disease or the pharmaceutical composition may further comprise such other active ingredients. For example, the peptide, salt thereof or composition can be used in combination with active ingredients used in the treatment of multiple sclerosis such as teriflunomide, interferon beta-la, interferon beta-lb, glatiramer acetate, fingolimod, mitoxantrone or corticosteroids. In an embodiment, the peptide, salt thereof or composition is used in combination with fingolimod.

The antagonistic peptide or salt thereof of the present disclosure can be useful for the treatment of cancer, and more particularly of PlexinA1/NRP1-expressing cancers. Indeed, it may be used to block Sema3A-dependent cancer cell migration, thereby preventing or reducing the occurrence of metastasis. In addition, as shown in the examples, the peptides are able to exhibit anti-angiogenic effect, thereby having a therapeutic effect on cancer. (Albrecht et al, Frontiers in Oncology, 2020, 10, Article 519). Disruption of NRP1/PlexA1 heterodimerization has been previously shown to block the pro-angiogenic activity of PlexinA1 and to inhibit tumor growth (Jacob et al, 2016, Oncotarget, 7, 57851-57865).

Accordingly, the present disclosure relates to the peptide, salt thereof or composition as described herein for use in the treatment of cancer (a PlexinA1/NRP1-expressing cancer), to the use of the peptide, salt thereof or composition as described herein for the manufacture of a medicament for the treatment of cancer (a PlexinA1/NRP1-expressing cancer), and to a method for the treatment of cancer (a PlexinA1/NRP1-expressing cancer) in a subject, comprising administering a therapeutically efficient amount of the peptide, salt thereof or composition as described herein to the subject. The therapeutic effect of the peptide may involve a decrease of the occurrence of metastasis, in particular by reducing cancer cell migration, a decrease of the tumor growth and/or a decrease of the angiogenesis.

The cancer can be selected from a hematopoietic cancer or a solid tumor, preferably solid tumor. Examples of cancer include, but are not limited to, solid tumors and hematological cancers, including carcinoma, lymphoma, blastoma (including medulloblastoma and retinoblastoma), sarcoma (including liposarcoma and synovial cell sarcoma), neuroendocrine tumors (including carcinoid tumors, gastrinoma, and islet cell cancer), mesothelioma, schwannoma (including acoustic neuroma), meningioma, adenocarcinoma, melanoma, and leukemia or lymphoid malignancies. More particular examples of such cancers include squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric cancer including gastrointestinal cancer, pancreatic cancer, glioblastoma, neuroblastoma, cervical cancer, ovarian cancer, liver cancer, bladder cancer, urinary tract cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, testicular cancer, esophageal cancer, tumors of the biliary tract, as well as head and neck cancer. Optionally, the cancer can be a cancer overexpressing PlexinA1 such as glioblastoma or gastric cancer. In a preferred aspect, the cancer is a CNS cancer, such as pilocytic astrocytomas, diffuse astrocytomas, anaplastic astrocytomas, glioblastomas, oligodendroglial tumors, ependymal tumor, medulloblastomas, pineal tumors, meningeal tumors and germ cell tumors, especially a glioblastoma.

In addition, the peptide, salt thereof or composition can be used in combination with other active ingredients or therapy used for the treatment of cancer or the pharmaceutical composition may further comprise such other active ingredients. Examples of active ingredients or therapy used for the treatment of cancer include chemotherapy (e.g., vinca alkaloids, agents that disrupt microtubule formation (such as colchicines and its derivatives), anti-angiogenic agents, therapeutic antibodies, EGFR targeting agents, tyrosine kinase targeting agent (such as tyrosine kinase inhibitors), transitional metal complexes, proteasome inhibitors, antimetabolites (such as nucleoside analogs), alkylating agents, platinum-based agents, anthracycline antibiotics, topoisomerase inhibitors, macrolides, retinoids (such as all-trans retinoic acids or a derivatives thereof), geldanamycin or a derivative thereof (such as 17-AAG), surgery, immune checkpoint inhibitors or immunotherapeutic agents (e.g., PD-1/PD-L1 inhibitors such as anti-PD-1/PD-L1 antibodies, CTLA-4 inhibitors such as anti-CTLA-4 antibodies, B7-1/B7-2 inhibitors such as anti-B7-1/B7-2 antibodies, TIM3 inhibitors such as anti-TIM3 antibodies, BTLA inhibitors such as anti-BTLA antibodies, CD47 inhibitors such as anti-CD47 antibodies, GITR inhibitors such as anti-GITR antibodies), antibodies against tumor antigens (e.g., anti-CD19, anti-CD22 antibodies), cell-based therapies (e.g., CAR T cells, CAR NK cells), and cytokines such as IL-2, IL-7, IL-21, and IL-15.

In an additional aspect, the present disclosure relates to the peptide, salt thereof or composition as described herein for use in the treatment of a disease or disorder associated with abnormal angiogenesis, to the use of the peptide, salt thereof or composition as described herein for the manufacture of a medicament for the treatment of a disease or disorder associated with abnormal angiogenesis, and to a method for the treatment of a disease or disorder associated with abnormal angiogenesis in a subject, comprising administering a therapeutically efficient amount of the peptide, salt thereof or composition as described herein to the subject. The therapeutic effect of the peptide may involve the inhibition of angiogenesis.

As used herein the term “disease or disorder associated with abnormal angiogenesis” refers to diseases caused by the dysregulation of the processes mediating angiogenesis. In particular, abnormal angiogenesis associated disease refers to hemangiomas, psoriasis, Kaposi's sarcoma, endometriosis, atherosclerosis, hypertension, tumor growth, inflammation, rheumatoid arthritis, wet-form age-related macular degeneration (AMD), choroidal neovascularization, ocular or retinal neovascularization, and diabetic retinopathy. In a particular aspect, the disease or disorder associated with abnormal angiogenesis is selected among tumor growth and metastasis, hemangiomas, psoriasis, Kaposi's sarcoma, ocular neovascularization, rheumatoid arthritis, endometriosis, or atherosclerosis.

Inhibition of PlexinA1 and/or NRP1 has also been shown to be suitable to inhibit immune cell infiltration and reduce inflammation, and for the treatment of inflammatory and autoimmune diseases (see, e.g., EP 2 497 498 A1 and WO 2016/033699). Thus, in another aspect, the present disclosure relates to the peptide, salt thereof or composition as described herein for use in the treatment of an inflammatory or autoimmune disease or condition, to the use of the peptide, salt thereof or composition as described herein for the manufacture of a medicament for the treatment of an inflammatory or autoimmune disease or condition, and to a method for the treatment of an inflammatory or autoimmune disease or condition in a subject, comprising administering a therapeutically efficient amount of the peptide, salt thereof or composition as described herein to the subject. The therapeutic effect of the peptide may involve the inhibition of immune cell infiltration and/or inflammation.

In an embodiment, the inflammatory or autoimmune disease or condition is anemia (aplastic anemia, hemolytic anemia, autoimmune hemolytic anemia, idiopathic thrombocytopenia), autoimmune hepatitis, iridocyclitis, scleritis, uveitis, orchitis, idiopathic thrombocytopenia purpura, Basedow's disease, Hashimoto's thyroiditis, juvenile-onset diabetes, inflammatory bowel disease, Addison's disease, demyelinating encephalitis, multiple sclerosis, septic shock, arthritis, inflammatory bowel disease (IBD), cutaneous skin inflammation, diabetes, uveitis, diabetic retinopathy, age-related macular degeneration (AMD), retinopathy of prematurity, amyotrophic lateral sclerosis (ALS), age-related cognitive decline/Alzheimer's disease, stroke, atopic dermatitis, chronic rheumatoid arthritis, systemic lupus erythematosus, Sjogren's syndrome, or psoriasis.

Further aspects and advantages of the present invention will be disclosed in the following experimental section, which should be regarded as illustrative and not limiting the scope of the present application. A number of references are cited in the present specification; each of these cited references is incorporated herein by reference.

### EXAMPLES

### Example 1: Proximity Ligation Assay

To demonstrate the ability of Membrane Targeting Peptides (MTPs) to disrupt the heterodimerization of Neuropilin-1 and PlexinA1, cells were seeded on Lab-Tek Permanox slides overnight, and then treated with appropriate peptide for 1 h. After fixation with 1% PFA for 10 min, slices were permeabilized with PBS/0.1% Triton X-100. Primary antibodies (NRP1 from Evitria, 1:500; and Plexin-A1 from Abcam, ab23391, 1:200) were incubated overnight at 4° C. in PBS. The proximity ligation assay allowing to visualize receptor dimers (Neuropilin-1/Plexin-A1) was then performed according to the manufacturer's recommendations with the “detection orange” kit (Sigma). Quantification of the interactions (fluorescent dots at the cell surface) was performed using ImageJ software.

The results show that GUNGNIR induced a −60% reduction of the number of NRP1/PlexinA1 interactions. MIMMING induced a similar −51% reduction while control inactive peptide SURTR had no effect on the number of NRP1/PlexinA1 dimers (Table 1).

TABLE 1
Number of NRP1-PlexinA1 interactions per Oli-
neu cells treated with peptides at 10⁻⁷ M

| | Normalized interactions Nrp1- | | One way ANOVA |
|---|---|---|---|
| Treatment | PlexinA1/cell | SEM | (vs. vehicle) |
| vehicle | 100 | ±8.1 | — |
| GUNGNIR | 40.4 | ±4.1 | P < 0.0001 |
| MIMMING | 49.4 | ±4 | P < 0.0001 |
| SURTR | 96.8 | ±6 | n.s. |

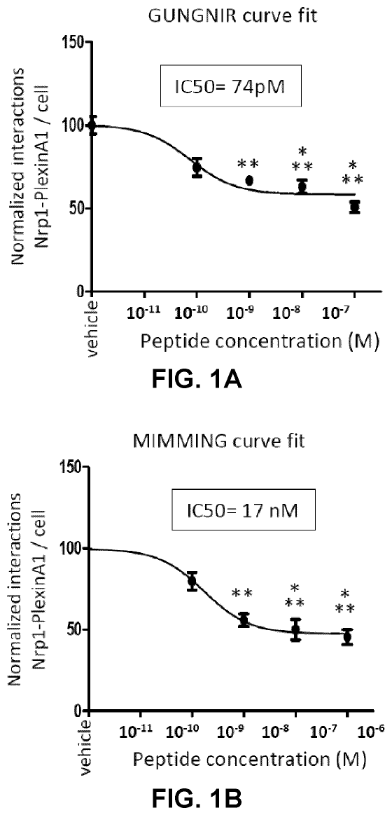

As seen in FIGS. 1A and 1B, both GUNGNIR and MIMMING showed dose-dependent effects in the number of NRP1/PlexinA1 interactions, with an IC₅₀ of 74 PM and 17 nM, respectively.

While demonstrating the ability of MTPs to disrupt NRP1/PlexinA1 dimers, these results show that MTPs display a sequence dependent blocking of NRP1/PlexinA1 receptor dimerization.

### Example 2: Cell Migration Assay

To demonstrate that MTPs rescue Sema3A negative effect on migration of the oligodendrocyte cell line Oli-neu, a cell migration assay using a Transwell CIM-Plate 16 (8 μm pore size filter ACEA Biosciences, Inc.) with xCELLigence RTCA DP Instrument (ACEA Biosciences Inc.) was performed. Cells were pre-incubated 1 h with vehicle alone or MTP. The 1×10⁵ cells were seeded in the upper chamber with 150 μl of medium. The bottom well contained 160 μl of medium supplemented with 2% fetal bovine serum for chemoattraction and 20 ng/ml Sema3A (Recombinant Mouse Semaphorin 3A Fc Chimera Protein Carrier Free ref: 5926-S3/CF; RnD Systems) for repulsion. Analysis was performed after 8 h of migration according to the manufacturer's instructions. Data are expressed as a percentage of positive control migration, i.e. the migration of Oli-neu with 2% serum and without Sema3A.

The results show that MTPs with different sequences rescued migration up to control migration thereby counteracting Sema3A inhibitory effect (FIG. 2A). Peptides with a G/S-X-X-X-G motif rescued migration, in contrast to peptides in which the G/S-X-X-X-G motif was replaced by G-X-X-X-S or S-X-X-X-A (n=3 excepted SKIRNIR n=1, FIG. 2B). Peptides with deletions next to G-X-X-X-G motif on N-terminal or C-terminal side rescued migration, but deletion of one G of the motif generated inactive peptides (FIG. 2C). N-ter shortening between (NterGp) and G-X-X-X-G motif impairs peptide efficiency (n=3 excepted BROKK and EITRI n=1, FIG. 2D). Among other peptides sequences with (CterpolyD/E), RATI, GUNGNIR and MIMMING rescue migration (n=3, FIG. 2E). Hence, in this functional assay, it was determined that GUNGNIR has an IC₅₀ of 1.2 nM (FIG. 2F). Finally, a similar activity was observed for MTP-PlexA1 (SEQ ID NO: 1) and its retro analog, ODIN, (SEQ ID NO:81) (FIG. 2G), providing evidence that retro analogs of the peptides described herein exhibit a biological activity similar to that of their corresponding peptides.

Overall, these result show that MTPs display a sequence dependent rescue of Sema3A inhibitory effect on oligodendrocytes migration. It depends on a minimal G/S-X-X-X-G motif (FIGS. 2A-C) and a suitable distance from (N-terGp) (FIG. 2D) and (C-terGp) (FIG. 2E).

### Example 3: Solubility Study

The solubility of the MTPs was tested either in phosphate buffered saline (PBS) or in a mixture of PBS and lithium dodecyl sulfate (LDS). The results are reported in Table 1:

TABLE 1
MTP solubility in PBS and PBS/LDS

| | | Solubilization | Measured |
|---|---|---|---|
| peptide | CterpolyD/E | vehicle | concentration |
| MTP-PlexA1 | no | PBS 100% | 0 |
| MTP-PlexA1 | no | PBS 100% LDS | 3.6 · 10⁻⁴M |
| | | 72 mM | |
| DRAUPNIR | yes | PBS 100% | 2.60 · 10⁻⁴M |
| RATI | yes | PBS 100% | 2.24 · 10⁻⁴M |
| GUNGNIR | yes | PBS 100% | 2.48 · 10⁻⁴M |
| HODR | yes | PBS 100% | 1.89 · 10⁻⁴M |

Failure to detect concentration via absorbance measurement shows that peptides without CterpolyD/E are not soluble in PBS 100% and need additional vehicle such as LDS, whereas peptides with CterpolyD/E are soluble in PBS 100%. This is more particularly exemplified by MTP-PlexA1 and DRAUPNIR which comprise the same sequence except for the C-terminal domain (MTP-PlexA1: TLPAIVGIGGGGGLLLLVIVAVLIAYKRK (SEQ ID NO: 1); DRAUPNIR: TLPAIVGIGGGGGLLLEVIVEVLIAYEEEEE (SEQ ID NO: 72).

### Example 4: Biodistribution Study

To address the bioavailability of MTPs in vivo, 3 CD1 8-week-old female mice were subjected to intraperitoneal injection of GUNGNIR-cy5 at the dose of 1, 10, 100 and 1000 μg/kg in a dose response experiment. Fluorescence was detected on gas-anesthetized animals, thanks to a Bioimager (Nightowl LB-983, Berthold), as previously described (Destouches et al. 2011, Cancer Res., 71 (9): 3296-305; Page et al. 2011, Ann Rheum Dis.; 70 (5): 837-43). The fluorescence was measured for each organ after sacrifice of the mice at 4 h. Acquisitions were performed during 10 s. for analysis, surface and intensity of the fluorescence was measured with the Nightowl program. As depicted in FIG. 3, GUGNIR is exhibiting large biodistribution profile including elimination organs (liver and kidney) in a dose dependent manner. Brain and spinal cord content was similar as the one measured in peripheric organs (heart, lung) showing efficient crossing of the blood brain barrier. Thus, GUNGNIR is able to reach the central nervous system and may be suitable for the treatment of neural diseases such as neurodegenerative diseases.

### Example 5: Induction and Assessment of Active Experimental Autoimmune Encephalomyelitis (EAE)

To assess the therapeutic potential of GUNGNIR, in vivo administration in mice suffering induced demyelination was performed. Mice were purchased from Janvier (8-9 weeks old when immunization is performed). All mice were fed in a controlled environment (25° C.) with free access to food and water and housed on a 12-h/12-h day/night cycle. Mice were paired-housed (equal number of each treatment per cage), and cages were changed weekly. All manipulations were performed in the morning. SJL/JRj female mice were used for EAE protocol with PLP immunisation and C57BL/6 female mice for EAE MOG immunisation. After 1 week of acclimatation to environment, mice were immunized with the kits developed by Hooke laboratories (EK-2120 or EK-2110) (during short anaesthesia with isoflurane). EAE PLP: Emulsion of PLP₁₃₉₋₁₅₁ fragment (HSLGKWLGHPDKF, SEQ ID NO: 74) in CFA (complete Freund's adjuvant) was administered as four subcutaneous injections of 50 μl according to the manufacturer's protocol. Mice received 0.4 μg of pertussis toxin intraperitoneally on the day of immunization. EAE MOG: Emulsion of MOG₃₅₋₅₅ fragment (MEVGWYRSPFSRVVHLYRNGK, SEQ ID NO: 75) in CFA (complete Freund's adjuvant) was administered as two subcutaneous injections of 100 μl according to the manufacturer's protocol. Mice received 0.4 μg of pertussis toxin intraperitoneally on the day of immunization and a second dose at day 1.

Peptide treatment started one day after immunization (EAE PLP) or 3 days after immunization (EAE MOG) relying on intraperitoneal administration of 100 μl of PBS or GUNGNIR diluted in PBS (10 μg/kg) three times per week (Monday/Wednesday/Friday). Clinical score was assessed daily from day 6 after immunization and was performed systematically before peptide injection. EAE was assessed clinically in blind conditions on a daily basis according to the following criteria: 0, no disease; 1, decreased tail tone; 2, impaired righting reflex and partial hind limb paresis; 3, complete hind limb paralysis; 4, hind limb paralysis with partial forelimb paralysis; and 5, moribund or dead.

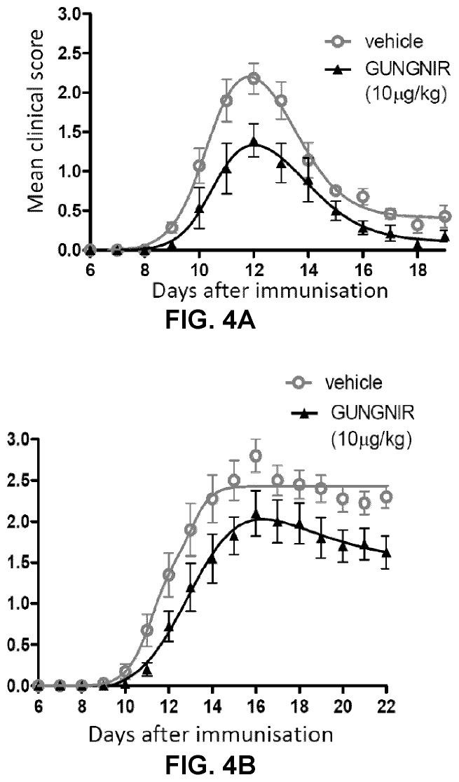

The analysis revealed a significant decrease of the clinical score when animal received 10 μg/kg GUNGNIR in the PLP (mimicking the recurrent-remitting form of Multiple Sclerosis) (FIG. 4A). Similarly, a significant reduction of the clinical score of mice receiving GUNGNIR was overserved in the EAE MOG model (Mimicking the progressive form of Multiple Sclerosis) (FIG. 4B).

Overall, these results show that GUNGNIR reduces the severity of EAE in PLP and MOG models.

### Example 6: Angiogenesis Assay

To address the clinical potential of MTPs targeting Plexin-A1 in angiogenesis related pathologies (including cancer and other diseases leading to abnormal vascularization), an angiogenesis assay measuring the ability of peptide to block angiogenesis was conducted. Human umbilical vein endothelial cells (HUVECs) were cultured at 37° C. under 5% CO₂ in endothelial cell culture medium (PromoCell, Heidelberg, Germany) supplemented with endothelial cell growth supplement (ECGS, 4 μL/mL), fetal calf serum (FCS, 20 μL/mL, Thermo Fisher Scientific), human epidermal growth factor (hEGF, 0.1 ng/ml), and human basic fibroblast growth factor (hbFGF, 1 ng/ml, Thermo Fisher Scientific). For the assay, plates (15μ-slide Angiogenesis, Ibidi plates, Biovalley, Nanterre, France) were coated with Matrigel (Merck-Millipore, Billerica, MA, USA) at 37° C. for 1 h. Subsequently, 5000 HUVECs in culture medium (50 μL) with peptide treatments were added to each well for 3 h (37° C., 5% CO₂). The cells in each well were imaged by DIC microscopy (Leitz DM RB, Leica, Nanterre, France) and the number of closed tubes was counted for 3 to 5 wells per condition.

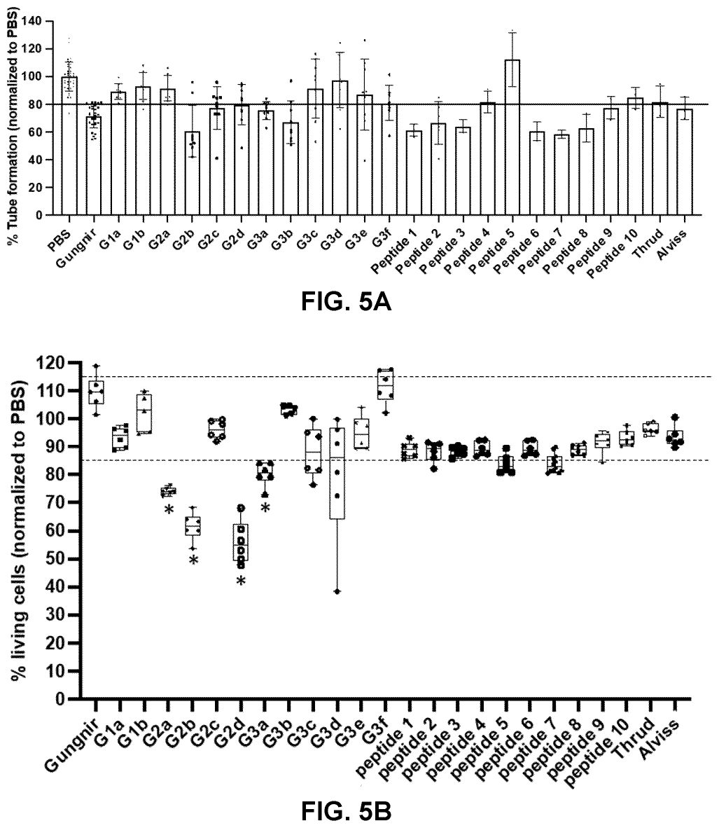

The results show the ability of GUNGNIR and RATI to inhibit angiogenesis, whereas the inactive control peptide HODR showed no effect in this assay (Table 2). Several analogs of GUNGNIR were also shown to inhibit angiogenesis (FIG. 5A).

TABLE 2
Inhibition of tubulogenesis

| | | Inhibition of | |
|---|---|---|---|
| Peptide | Concentration | tubulogenesis | SEM |
| GUNGNIR | 10⁻⁷ M | 44% | +/−6.6 |
| RATI | 10⁻⁷ M | 39.4% | +/−7.5 |
| HODR | 10⁻⁶ M | 4.5% | */−7.7 |

### Example 7: MTT Assay

The toxicity of MTPs was assessed using an MTT assay. HUVEC cells were used for this assay at a concentration of 20 000 cells in 100 μL by well (in a 96-well plate). After 24 hours of incubation at 37° C., the peptide, or its vehicle (PBS), was added at a concentration of 10-7M. After 4 hours of incubation at 37° C., the medium was removed and replaced by MTT (stock concentration at 5 mg/mL) diluted at 1/20 with Gey's Balanced Salt Solution (GBSS). After 4 hours of incubation at 37° C., the cells were lysed with 100 μL of isopropanol by wells and the plate was analyzed by spectrophotometry at the wavelength of 570 nm.

The results are reported in FIG. 5B.

### Example 8: Remyelination Study

### Experimental Design and Conditions

It was next assessed whether GUNGNIR can induce remyelination of the CNS and motor function recovery following demyelination induced by cuprizone, a copper-chelating agent, in a mouse model. Oral intoxication with cuprizone induces oligodendrocyte apoptosis within a few days, which is closely followed by the activation of the innate immune cells in the brain, i.e., astrocytes and microglia, finally leading to demyelination of distinct white and grey matter brain areas.

The following treatment groups were included in the study:
(a) control (n=5)
(b) 5 weeks cuprizone (to demonstrate demyelination; n=10)
(c) 5 weeks cuprizone, followed by treatment with vehicle for 6 consecutive days (n=10)
(d) 5 weeks cuprizone, followed by treatment with GUNGNIR (10 μg/kg) for 6 consecutive days (n=10)
(e) 5 weeks cuprizone, followed by treatment with GUNGNIR (100 μg/kg) for 6 consecutive days (n=10)
(f) 5 weeks cuprizone, followed by treatment with vehicle for 11 consecutive days (n=10)
(g) 5 weeks cuprizone, followed by treatment with GUNGNIR (10 μg/kg) for 11 consecutive days (n=10)
(h) 5 weeks cuprizone, followed by treatment with GUNGNIR (100 μg/kg) for 11 consecutive days (n=10)

Acute demyelination was induced by intoxicating for five consecutive weeks ˜8-week-old (19-21 g) male mice a diet containing 0.25% cuprizone [bis(cyclohexanone)oxaldihydrazone; Sigma-Aldrich Inc., St Louis, MO, USA] mixed into a ground standard rodent chow. Treatment with Vehicle or GUNGNIR compound (10 and 100 μg/kg) was performed by intra peritoneal (i.p.)

injection treatment three times/week, starting after three weeks of cuprizone administration (i.e., at the beginning of week 4) till the end of the experiment (i.e., after 6 or 11 days remyelination).

Assessment of myelination was performed by immunohistochemistry and histochemistry. Myelin marker proteolipid protein (PLP), the major myelin protein within the central nervous system, was visualized by immunohistochemistry using the following antibody: Bio-Rad Cat #MCA839G, RRID: AB_2237198, 1:5000. Intact and damaged myelin was additionally visualized using luxol-fast-blue (LFB)/periodic acid-Shiff (PAS) histochemical stains.

Assessment of locomotor function (Gait analysis) was performed using High-Speed Ventral Plane Videography. Gait analyses were performed one time before termination of the experiment on all experimental mice using the DigiGait™ imaging system along with the DigiGait™ 15.0 analysis software (Mouse Specifics, Inc.; Quincy MA) as previously described (Zhan et al., 2019)

Differences between the individual experimental groups were statistically tested with appropriate multiple comparisons. For the gait analyses groups d-e (i.e., 6 days remyelination) and f-h (i.e. 11 days remyelination) were tested separately for normal data distribution by using the Kolmogorov-Smirnov test. Afterwards, ordinary One-Way-ANOVA, followed by Dunnett's multiple comparisons test was performed for parametric data whereas Kruskal-Wallis Test followed by Dunn's multiple comparisons test was performed for non-parametric data. In each case, multiple comparisons were performed by comparing the vehicle versus the low dose and the high dose groups.

For the histological LFB/PAS related data, non-parametric Kruskal-Wallis tests, followed by Dunns's multiple comparison test were performed separately for 6 days and 11 days remyelination. For the immunohistological anti-PLP related data, parametric ordinary one-way ANOVA, followed by Dunnett's multiple comparison test were performed separately for 6 days and 11 days remyelination. All results are shown as mean±SEM.

Weights between the groups were tested using either ordinary One-Way-ANOVA, followed by Dunnett's multiple comparisons test or non-parametric Kruskal-Wallis tests, followed by Dunn's multiple comparison test.

### Results

### A. Gait Analysis

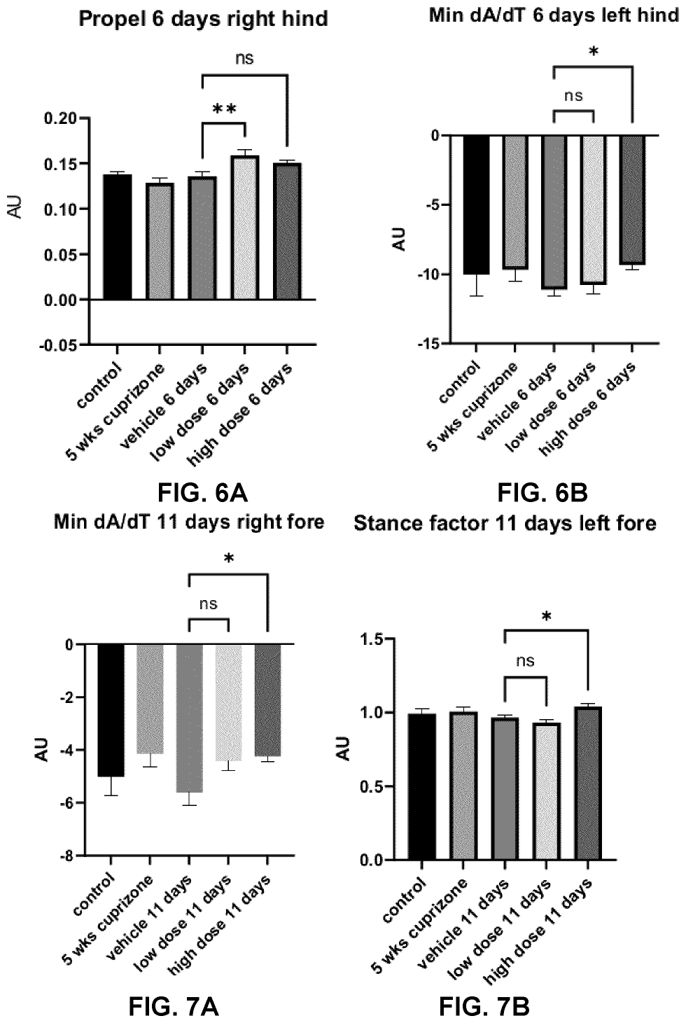

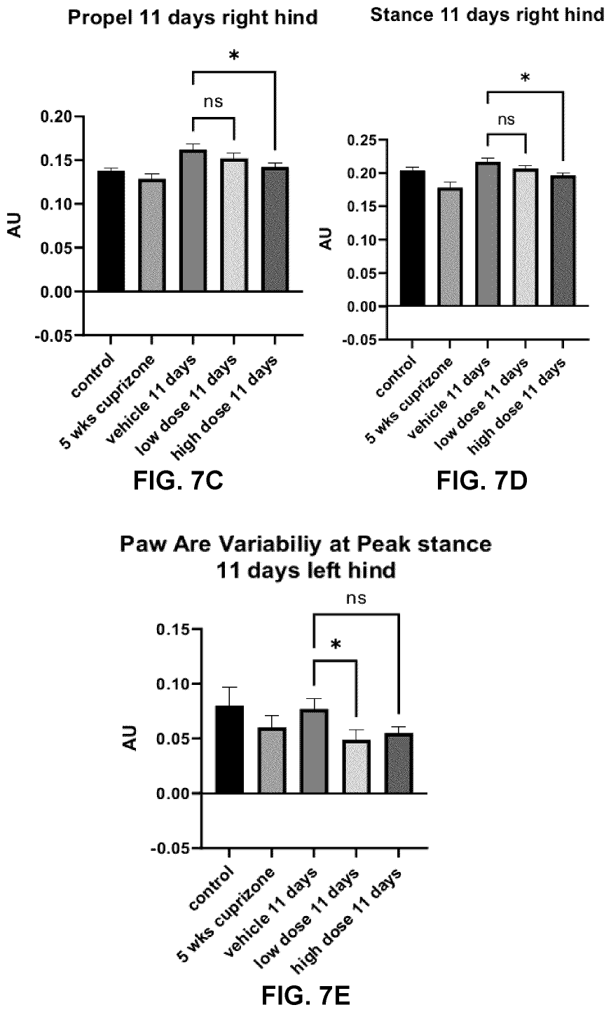

A maximum of 41 different gait metrics were evaluated by the DigiGait™ imaging software, separately for each paw (id est, left fore, right fore, left hind and right hind). As summarized in FIGS. 6A-B, after 6 days of remyelination, 1 gait parameter was significantly different between vehicle and 10 μg/kg groups and 1 gait parameter was significantly different between vehicle and 100 μg/kg groups. As summarized in FIGS. 7A-E, after 11 days of remyelination, 1 gait parameter was significantly different between vehicle and 10 μg/kg groups and 4 gait parameters were significantly different between vehicle and 100 μg/kg groups.

### B. Histology

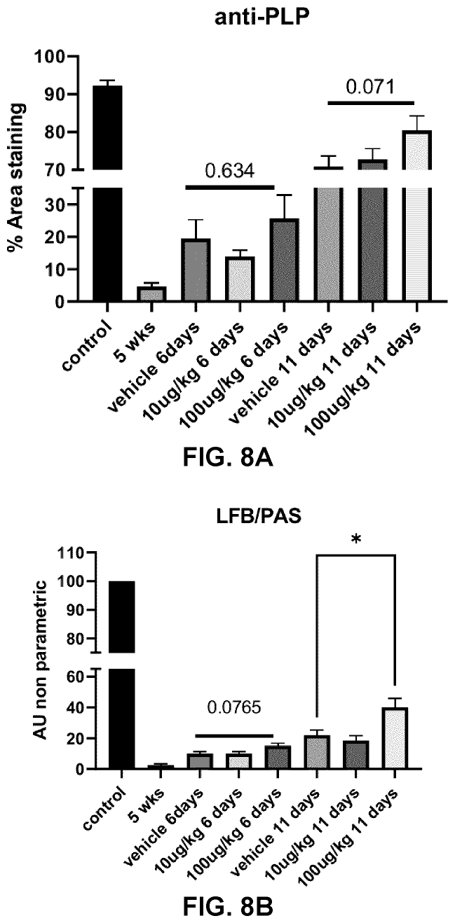

As demonstrated in FIG. 8A, high anti-PLP staining intensities were found in control mice with a pronounced decrease in 5 wks cuprizone-treated mice (5 wks). Staining intensities increased during the 6 and 11 days remyelination periods. At day 11, anti-PLP staining intensities were by trend higher in 100 μg/kg relative to vehicle groups (p=0.071). The same staining patterns were observed with LFB-processed sections. As demonstrated in FIG. 8B, high LFB staining intensities were found in control mice which a pronounced decrease in 5 wks cuprizone-intoxicated mice (5 wks). Again, staining intensities recovered, however to a slower range. Of note, at day 11 LFB staining intensities were significantly higher in 100 μg/kg relative to vehicle groups (22±3.3 in vehicle versus 40±5.8 in 100 μg/kg treated mice).

### C. Body Weight

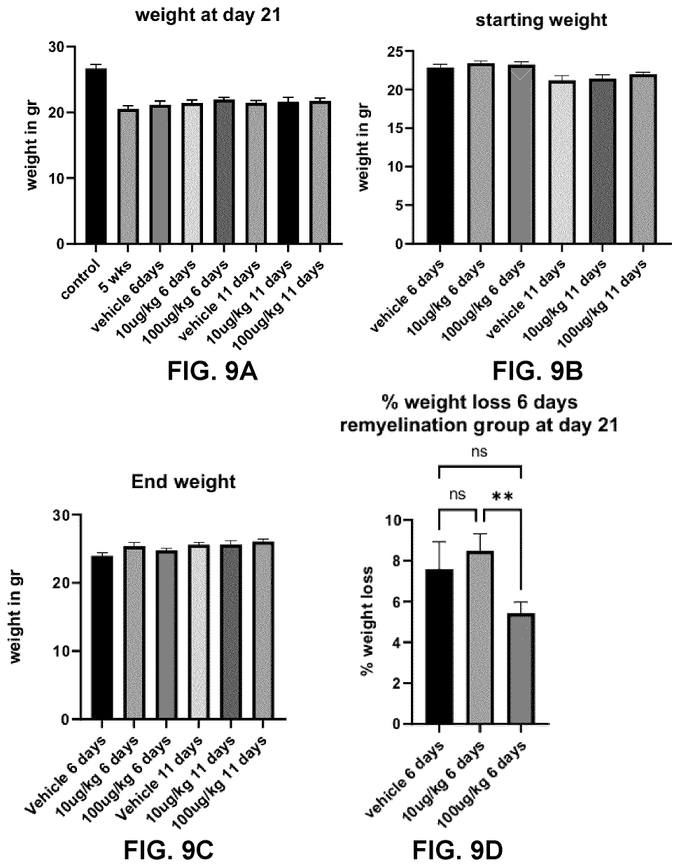

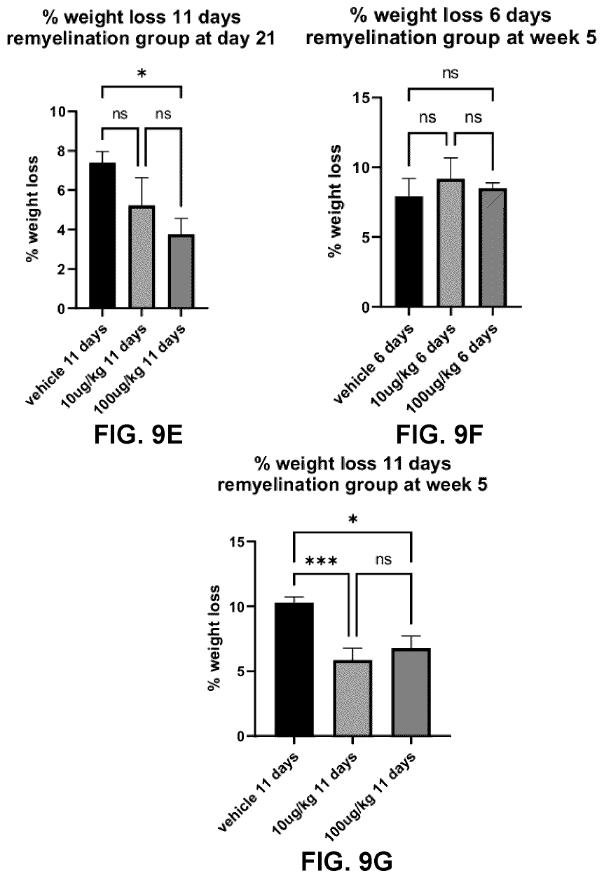

Body weights were assessed at different time point of the experiment. As demonstrated in FIG. 9A, before initiation of the cuprizone treatment (i.e., at day 21), there was no significant difference of the body weight of mice between the cuprizone-treated groups. One-Way-ANOVA, followed by Dunnett's multiple comparisons test (comparing separately 6 days and 11 days groups among each other). Comparably, as shown in FIG. 9B and FIG. 9C, no differences were observed at the beginning of the experiment and at the day of the termination of the individual groups treated with GUNGNIR. As demonstrated in FIGS. 9D-G, the percentage loss of body weight at weeks 3 and 5 was significantly less pronounced in some of the GUNGNIR-treated groups relative to the vehicle-treated groups

### Example 9: Combination Therapy in the Recurrent Remittent Experimental Autoimmune Encephalomyelitis (EAE-PLP) Model

### Experimental Design and Conditions

The effect of GUNGNIR, Fingolimod and a combination of Fingolimod with GUNGNIR was studied in the recurrent remittent Experimental Autoimmune Encephalomyelitis (EAE-PLP) mouse model. Treatments were administrated 3 times per week at 10 μg/kg for GUNGNIR intraperitoneally (IP) in PBS, and daily from D12 (peak) at 1 mg/kg IP for Fingolimod. Results were compared to a control group treated 3 times per week with vehicle (PBS and then PBS with 25% ethanol from D12). Treatments were started at D2 post-induction (D1) for GUNGNIR and at D12 post-induction for Fingolimod. Treatments were administrated for 5 weeks. Score were evaluated daily from D7, as well as body weight.

Mice were anesthetized with 3% isoflurane an induced with Hooke Kit™ [ser140]-PLP139-151/CFA Emulsion PTX (cat. no. EK-2120) according to the manufacturer's instruction. In brief, mice were subcutaneously injected in the left and right hip and left and right shoulder with 0.05 ml of PLP emulsion each (meaning 0.2 ml total per mouse). Then, a solution of pertussis toxin (PTX) was injected intraperitoneally, 30 ng per mouse according to the concentration of the pertussis batch.

### Results

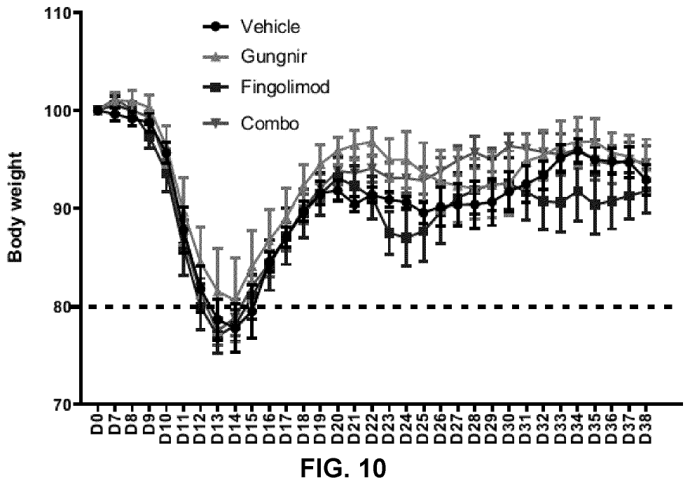

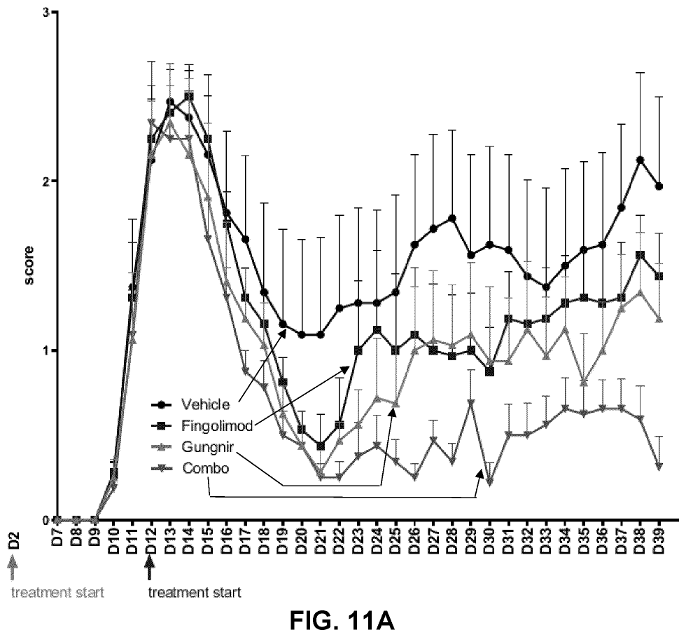

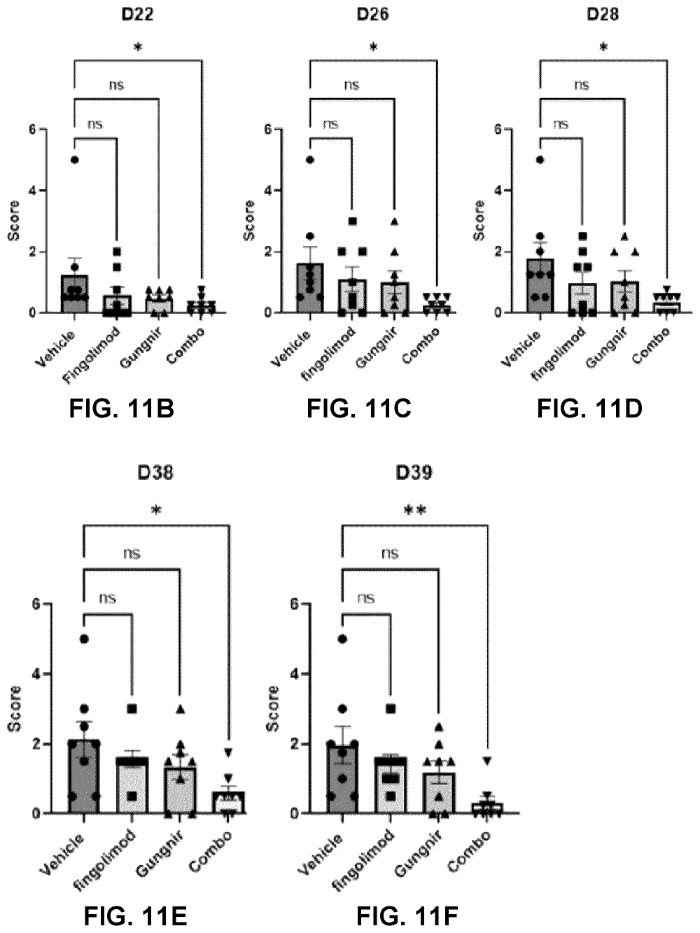

During the time of the experiment, all mice showed a reduction of body weight not reaching the ethical limit point (−20%). All groups are exhibiting a similar course of the disease therefore allowing comparisons (FIG. 10). None of the treatments had an effect on peak intensity which was similar in all experimental groups (FIG. 11A). However, the three treated groups showed a significant recovery relative to the control group (FIG. 11A). The amplitude of the effect was similar for all treatments but the GUNGNIR+Fingolimod treatment (combo) showed a better efficacy over time (FIG. 11A). From D24, Fingolimod or GUGNIR in stand-alone showed intermediate scoring while the GUNGNIR+Fingolimod combination displayed an almost full protective effects with scores not reaching 1 (FIG. 11A). The analysis of individual scores confirmed that the GUNGNIR+Fingolimod combination provides the most efficient therapeutic effect with less variability as seen for the stand-alone conditions (FIGS. 11B-F).

### Example 10: Combination Therapy in the Progressive Experimental Autoimmune Encephalomyelitis (EAE-MOG) Model

### Experimental Design and Conditions

The effect of GUNGNIR, Fingolimod and a combination of Fingolimod with GUNGNIR was studied in the progressive Experimental Autoimmune Encephalomyelitis (EAE-MOG) mouse model. Treatments were administrated 3 times per week at 10 μg/kg for GUNGNIR intraperitoneally (IP) in PBS, and daily from D14 at 1 mg/kg IP for Fingolimod. Results were compared to a control group treated 3 times per week with vehicle (PBS and then PBS with 25% ethanol from D14). Treatments were started at D3 post-induction (D1) for GUNGNIR and at D14 post-induction for Fingolimod. Treatments were administrated for 5 weeks. Score were evaluated daily from D7, as well as body weight.

Mice were anesthetized with 3% isoflurane an induced with Hooke Kit™ MOG35-55/CFA Emulsion PTX (cat. no. EK-2110) according to the manufacturer's instruction In brief, mice were subcutaneously injected in the upper back with 0.1 ml of MOG emulsion, and similarly in the lower back with 0.1 ml of emulsion. After 2 hours, a solution of pertussis toxin (PTX) was injected intraperitoneally, 80 ng per mouse. A second IP injection of the same amount of PTX was given 24 h later.

### Results

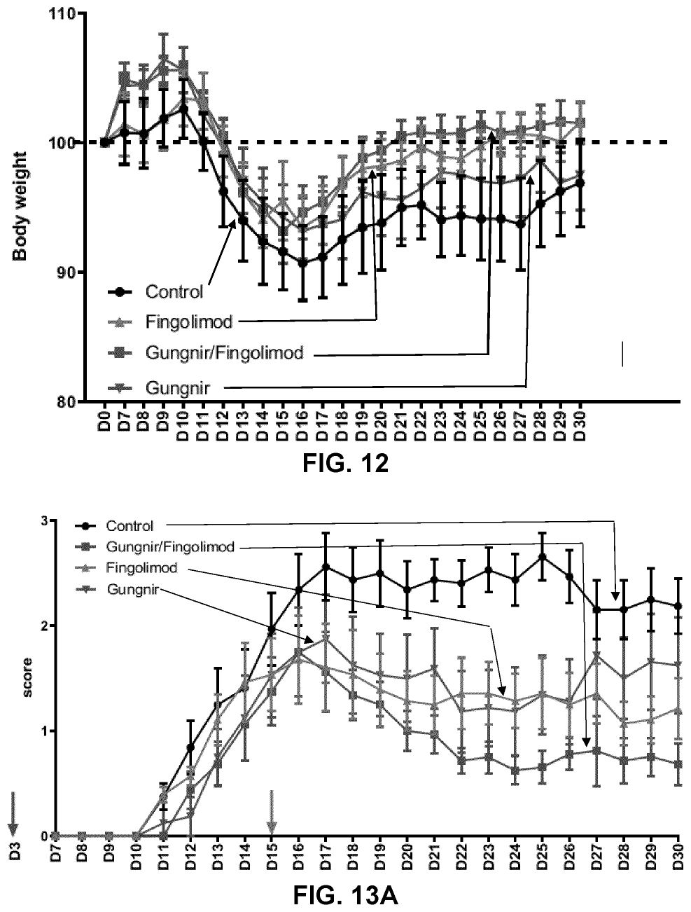

During the time of the experiment, all mice showed a reduction of body weight not reaching the ethical limit point (−20%). All groups are exhibiting a similar course of the disease therefore allowing comparisons (FIG. 12).

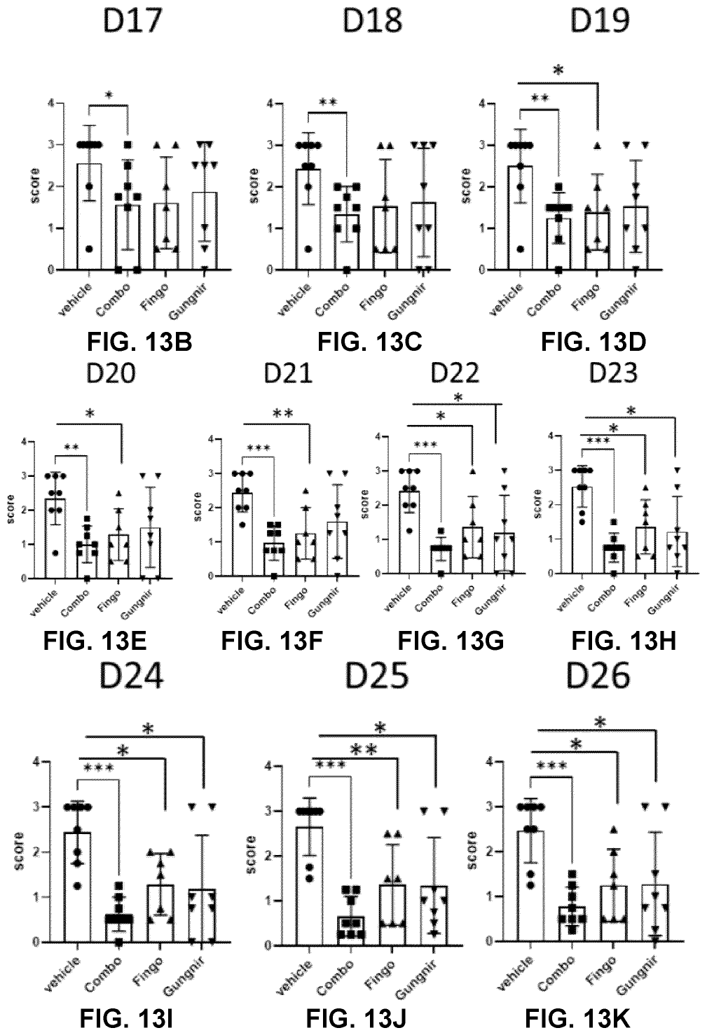

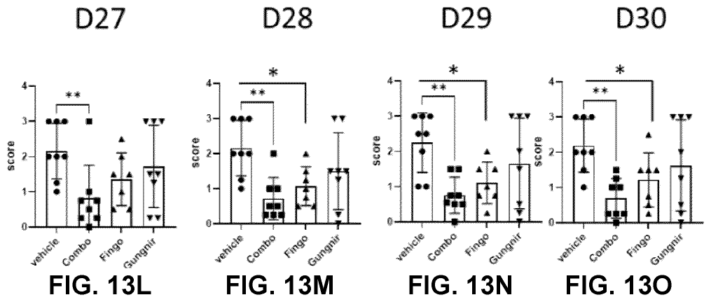

In this experiment, the control group reached a peak of the disease at D17. All animals in the control group showed persistence of the disease during the remaining 13 days of the protocol (FIG. 13A). Strikingly, all treated animals showed a reduction of the disease severity from D15. The therapeutic effect was stronger for the GUNGNIR+Fingolimod combination with scores below 1 from D22 up to the last day of the study. Fingolimod or GUGNIR in stand-alone showed intermediate scoring. The analysis of individual scores confirmed that the GUNGNIR+Fingolimod combination shows the best therapeutic effect (FIGS. 13B-O). Interestingly, this group also displayed the smaller interindividual variability.

### Peptide Sequences

| | |
|---|---|
| BALDR | KGDWLPAITGLVGGVGLL (SEQ ID NO: 52) |
| FREYR | KGDWLPAIVSIGGGVVLL (SEQ ID NO: 53) |
| BRAGI | KGDWIPALVGGGGGGGLL (SEQ ID NO: 54) |
| NJORD | KGDWLPALVSIGGGVGLL (SEQ ID NO: 55) |
| ULLR | KGDWIPALVGLGGGGGLL (SEQ ID NO: 56) |
| SKOLL | KGDWLIAIVGIGGG (SEQ ID NO: 57) |
| HATI | KGDWLPVIVGIGGG (SEQ ID NO: 58) |
| plexA1-S2 | KGDWLPAIVGIGGGGGLL (SEQ ID NO: 59) |
| FJALAR | KGDWLPAIVGIGGGGGL (SEQ ID NO: 60) |
| GALAR | KGDWLPAIVGIGGGGG (SEQ ID NO: 61) |
| IVALDI | KGDWLPAIVGIGGGG (SEQ ID NO: 62) |
| ALVISS | KGDWLPAIVGIGGG (SEQ ID NO: 63) |
| KVASIR | AITGLVGGVGLLLEVIVEVAYEEEEE (SEQ ID NO: 16) |
| GERD | TGLVGGVGLLLEVIVEVAYEEEEE (SEQ ID NO: 17) |
| THRUD | GLVGGVGLLLEVIVEVAYEEEEE (SEQ ID NO: 18) |
| RATI | TLPAIVGIGGGGGELLLVIVEVLIYEEEEE (SEQ ID NO: 19) |
| GUNGNIR | TLPAITGLVGGVGLLLEVIVEVAYEEEEE (SEQ ID NO: 20) |
| MIMMING | TLPAIVSIGGGVVLLLEVIVEVAYEEEEE (SEQ ID NO: 21) |
| SURTR | TLPVIGVIVLVGSAVLELIAEGVYEEEEE (SEQ ID NO: 64), |
| | scramble of MIMMING |
| HODR | TLPAIGVITLVGLGVLELVAEGVYEEEEE (SEQ ID NO: 65), |
| | scramble of GUNGNIR |
| GULLVEIG | KGDWLPAIVSIGGAVVLL (SEQ ID NO: 66) |
| SKIRNIR | KGDWLPAIVGIGGSVVLL (SEQ ID NO: 67) |
| RATATOSK | KGDWLPAIVGIGG (SEQ ID NO: 68) |
| plexA1-S1 | KGDWTLPAIVGIGGGGGLL (SEQ ID NO: 69) |
| BROKK | KGDWPAIVGIGGGGGLL (SEQ ID NO: 70) |
| EITRI | KGDWIVGIGGGGGLL (SEQ ID NO: 71) |
| DRAUPNIR | TLPAIVGIGGGGGLLLEVIVEVLIAYEEEEE (SEQ ID NO: 72) |
| MJOLLNIR | TLPAITGLVGGVGLLVEVAVEIAYEEEEE (SEQ ID NO: 73) |
| G1a | TLPAITGVVVGGVGLLLEVIVEVAYEEEEE (SEQ ID NO: 94) |
| G1b | TLPAITGLVVGVGLLLEVIVEVAYEEEEE (SEQ ID NO: 95) |
| G2a | TLPAITGLVGGVGLLLEVIVEVAYEEEE (SEQ ID NO: 96) |
| G2b | TLPAITGLVGGVGLLLEVIVEVAYEEE (SEQ ID NO: 97) |
| G2c | TLPAITGLVGGVGLLLEVIVEVAYEE (SEQ ID NO: 98) |
| G2d | TLPAITGLVGGVGLLLEVIVEVAYDDDDD (SEQ ID NO: 99) |
| G3a | TLPAITGLVGGVVLLLEVIVEVAYEEEEE (SEQ ID NO: 100) |
| G3b | TLPAITGLVGGVGLVLEVIVEVAYEEEEE (SEQ ID NO: 101) |
| G3c | TLPAITGLVGGVGLLLVVIVEVAYEEEEE (SEQ ID NO: 102) |
| G3d | TLPAITGLVGGVGLLLEVVVEVAYEEEEE (SEQ ID NO: 103) |
| G3e | TLPAITGLVGGVGLLLEVIVVVAYEEEEE (SEQ ID NO: 104) |
| G3f | TLPAITGLVGGVGLLLEVIVEVVYEEEEE (SEQ ID NO: 105) |
| Pep1 | _{d}E_{d}E_{d}E_{d}E_{d}E_{d}Y_{d}A_{d}V_{d}E_{d}V_{d}I_{d}V_{d}E_{d}L_{d}L_{d}LG_{d}VGG_{d}V_{d}LG_{d}T_{d}I_{d}A_{d}P_{d}L_{d}T |
| | (SEQ ID NO: 106) |
| Pep2 | TLPAITGLVGGVGLLLEVIVEVAYDD (SEQ ID NO: 107) |
| Pep3 | TLPAITGLVGGVGLLLEVIVEVAYDEDED (SEQ ID NO: 108) |
| Pep4 | TGLVGGVGLLLEVIVEVAYEEEE (SEQ ID NO: 109) |
| Pep5 | TGLVGGVGLLLEVIVEVAYEEE (SEQ ID NO: 110) |
| Pep6 | _{d}TLPAITGLVGGVGLLLEVIVEVAYEEEEE (SEQ ID NO: 111) |
| Pep7 | TLPAITGLVGGVGLLLEVIV_{d}EVAYEEEEE (SEQ ID NO: 112) |
| Pep8 | TLPAITGLVGGVGLLVEVIVEVAYEEEEE (SEQ ID NO: 113) |
| Pep9 | TLPAITGLVGGVGLLLEVAVEVAYEEEEE (SEQ ID NO: 114) |
| Pep10 | TLPAITGLVGGVGLLLEVIVEIAYEEEEE (SEQ ID NO: 115) |

1-68. (canceled)

69. A peptide comprising:
a first domain of the sequence X37-X5-X6-X7-G, and
(i) a second domain of the formula I: -(X26-X27-X28-X29-X30-X31-X32-X33-X34-X35-X36)-(CterpolyD/E) directly linked at carboxy-terminal end of the first domain;
(ii) a second domain of the formula II: -(X13-X14-X15-X16-X17-X18-X19-X20-X21-X22-X23-X24-X25)-(CterGp) directly linked at carboxy-terminal end of the first domain; or
(iii) a second domain of the formula III: (NterGp)-(X8-X9-X10-X11-X12)-directly linked at amino-terminal end of the first domain;
wherein
X37 is Gly, L-Ser or D-Ser;
X5, X6 and X7 are independently any amino acid;
X8, X9, X10, X11 and X12 are independently any amino acid,
X13, X14, X15, X16, X17, X18, X19, X20, X21, X22, X23, X24 and X25 are independently any amino acid, wherein no more than 2 amino acids among X13, X14, X15, X16, X17, X18, X19, X20, X21, X22, X23, X24 and X25 are a charged amino acid;
X26, X27, X28, X29, X30, X31, X32, X33, X34, X35 and X36 are independently any amino acid, wherein no more than 2 amino acids among X26, X27, X28, X29, X30, X31, X32, X33, X34, X35 are a charged amino acid;
CterpolyD/E is a group of 4-10 amino acids including at least 2 negatively charged amino acids;
NterGp is a group of 3-5 amino acids including at least 2 charged amino acids, and
CterGp is a group of 3-5 amino acids including at least 3 positively charged amino acids,
or a retro or retroinverso form thereof,
or a pharmaceutically acceptable salt thereof.

70. The peptide of claim 69, wherein X5, X6 and X7 are aliphatic uncharged amino acids.

71. The peptide of claim 70, wherein X5, X6 and X7 are each independently Gly, D or L-Ala, D or L-Val, D or L-Leu and D or L-Ile; and/or X37 is Gly.

72. The peptide of claim 71, wherein X5 is D-Leu, L-Leu, D-Ile or L-Ile; X6 is Gly, D-Val or L-Val; and/or X7 is Gly.

73. The peptide of claim 69, wherein X26 is an aliphatic uncharged amino acid; X27 is an aliphatic uncharged amino acid; X28 is an aliphatic uncharged amino acid; X29 is an aliphatic uncharged amino acid; X30 is an aliphatic uncharged amino acid; X31 is a negatively charged amino acid; X32 is an aliphatic uncharged amino acid; X33 is an aliphatic uncharged amino acid; X34 is an aliphatic uncharged amino acid; X35 is a negatively charged amino acid; and/or X36 is an aliphatic uncharged amino acid.

74. The peptide of claim 73, wherein X26 is Gly, D-Val or L-Val; X27 is Gly, D-Val or L-Val; X28 is D or L-Ala, D- or L-Val, D- or L-Leu, or D- or L-Ile; X29 is D- or L-Ala, D- or L-Val, D- or L-Leu, or D- or L-Ile; X30 is D- or L-Ala, D- or L-Val, D- or L-Leu, or D- or L-Ile; X31 is D or L-Glu; X32 is D- or L-Ala, D- or L-Val, D- or L-Leu, or D- or L-Ile; X33 is D- or L-Ala, D- or L-Val, D- or L-Leu, or D- or L-Ile; X34 is D- or L-Ala, D- or L-Val, D- or L-Leu, or D- or L-Ile; X35 is D or L-Glu; and/or X36 is D- or L-Ala, D- or L-Val, D- or L-Leu, or D- or L-Ile.

75. The peptide of claim 74, wherein X26 is L-Val; X27 is Gly; X28 is L-Leu; X29 is L-Leu; X30 is L-Leu; X31 is L-Glu; X32 is L-Val; X33 is L-Ile; X34 is L-Val; X35 is L-Glu; and/or X36 is L-Val.

76. The peptide of claim 74, wherein X26-X27-X28-X29-X30-X31-X32-X33-X34-X35-X36 is VGLLLEVIVEV (SEQ ID NO: 91), GGELLLVIVE (SEQ ID NO: 92), VVLLLEVIVEV (SEQ ID NO: 93), VGLLVEVIVEV (SEQ ID NO:117), or VGLVLEVIVEV (SEQ ID NO:118).

77. The peptide of claim 69, wherein CterpolyD/E comprises a sequence of -X″1-X″2-Z or -X″3-X″3bis-X″2-Z, wherein
X″1 is a small amino acid,
X″2 is an aromatic amino acid,
X″3 and X″3bis are independently long aliphatic amino acids, and
Z is from 2 to 10 D/L-Asp and/or D/L-Glu residues.

78. The peptide of claim 77, wherein X″1 is D- or L-Ala; and/or X″3 and X″3bis are independently D- or L-Leu, or D- or L-Ile.

79. The peptide of claim 77, wherein Z is from 3 to 6 D/L-Asp and/or D/L-Glu residues.

80. The peptide of claim 79, wherein Z is from 3 to 5 L-Glu residues.

81. The peptide of claim 69, wherein the peptide comprises a second domain of formula I or II, and further comprises a third domain of 1 to 10 amino acids linked at the amino-terminal end of the first domain.

82. The peptide of claim 81, wherein the third domain is of the formula IV:
X38-X39-X40-X41-X42-X43, wherein
X38 is D-Thr or L-Thr, or is absent;
X39 is D- or L-Leu, D- or L-Ile, or is absent;
X40 is Pro, D- or L-Ile, or is absent;
X41 is D- or L-Ala, D- or L-Val, or is absent;
X42 is D- or L-Leu, D- or L-Ile, or is absent; and
X43 is D- or L-Val, D-Thr or L-Thr.

83. The peptide of claim 69, which comprises 35 or 30 amino acid or less.

84. The peptide of claim 69, which comprises one of the following sequences:
(SEQ ID NO: 16)
AITGLVGGVGLLLEVIVEVAYEEEEE;
(SEQ ID NO: 17)
TGLVGGVGLLLEVIVEVAYEEEEE;
(SEQ ID NO: 18)
GLVGGVGLLLEVIVEVAYEEEEE;
(SEQ ID NO: 19)
TLPAIVGIGGGGGELLLVIVEVLIYEEEEE;
(SEQ ID NO: 20)
TLPAITGLVGGVGLLLEVIVEVAYEEEEE;
(SEQ ID NO: 21)
TLPAIVSIGGGVVLLLEVIVEVAYEEEEE;
(SEQ ID NO: 97)
TLPAITGLVGGVGLLLEVIVEVAYEEE;
(SEQ ID NO: 98)
TLPAITGLVGGVGLLLEVIVEVAYEE;
(SEQ ID NO: 99)
TLPAITGLVGGVGLLLEVIVEVAYDDDDD;
(SEQ ID NO: 100)
TLPAITGLVGGVVLLLEVIVEVAYEEEEE;
(SEQ ID NO: 101)
TLPAITGLVGGVGLVLEVIVEVAYEEEEE;
(SEQ ID NO: 105)
TLPAITGLVGGVGLLLEVIVEVVYEEEEE;
(SEQ ID NO: 106)
_{d}E_{d}E_{d}E_{d}E_{d}E_{d}Y_{d}A_{d}V_{d}E_{d}V_{d}I_{d}V_{d}E_{d}L_{d}L_{d}LG_{d}VGG_{d}V_{d}LG_{d}T_{d}I_{d}A_{d}P_{d}L_{d}T;
(SEQ ID NO: 107)
TLPAITGLVGGVGLLLEVIVEVAYDD;
(SEQ ID NO: 108)
TLPAITGLVGGVGLLLEVIVEVAYDEDED;
(SEQ ID NO: 111)
_{d}TLPAITGLVGGVGLLLEVIVEVAYEEEEE;
(SEQ ID NO: 112)
TLPAITGLVGGVGLLLEVIV_{d}EVAYEEEEE;
or
(SEQ ID NO: 113)
TLPAITGLVGGVGLLVEVIVEVAYEEEEE.

85. The peptide of claim 84, which comprises one of the following sequences:
(SEQ ID NO: 20)
TLPAITGLVGGVGLLLEVIVEVAYEEEEE;
(SEQ ID NO: 100)
TLPAITGLVGGVVLLLEVIVEVAYEEEEE;
(SEQ ID NO: 108)
TLPAITGLVGGVGLLLEVIVEVAYDEDED;
or
(SEQ ID NO: 113)
TLPAITGLVGGVGLLVEVIVEVAYEEEEE.

86. A method for treating a demyelinating disease in a subject in need thereof, the method comprising administering to the subject an effective amount of the peptide, retro or retroinverso form thereof, or pharmaceutically acceptable salt thereof according to claim 68, or a pharmaceutical composition comprising the peptide, retro or retroinverso form thereof, or pharmaceutically acceptable salt thereof.

87. The method of claim 86, wherein the demyelinating disease is a demyelinating autoimmune disease.

88. The method of claim 87, wherein the demyelinating autoimmune disease is multiple sclerosis.

89. The method of claim 86, wherein the method further comprises administering to the subject an effective amount of fingolimod.


SEQUENCE LISTING (119 sequences)

SEQ ID NO: 1 - protein, 29 aa, synthetic construct
  REGION 1..29: note = MTP-PlexA1
TLPAIVGIGGGGGLLLLVIVAVLIAYKRK

SEQ ID NO: 2 - protein, 16 aa, synthetic construct
  REGION 1..16: note = antagonistic peptide
  VAR_SEQ 1: note = Xaa is G or S
  VAR_SEQ 2: note = Xaa is I or G or L
  VAR_SEQ 3: note = Xaa is G or V
  VAR_SEQ 6..7: note = Xaa is G orV
  VAR_SEQ 8: note = Xaa is L or E
  VAR_SEQ 11: note = Xaa is L or E
  VAR_SEQ 15: note = Xaa is A or E
  MOD_RES 16: note = BOUND AT THE C TERM TO CterpolyE
XXXGGXXXLLXVIVXV

SEQ ID NO: 3 - protein, 17 aa, synthetic construct
  REGION 1..17: note = ANTAGONISTIC PEPTIDE
  VAR_SEQ 1: note = Xaa is V or T
  VAR_SEQ 2: note = Xaa is G or S
  VAR_SEQ 3: note = Xaa is I or G or L
  VAR_SEQ 4: note = Xaa is G or V
  VAR_SEQ 7..8: note = Xaa is G or V
  VAR_SEQ 9: note = Xaa is L or E
  VAR_SEQ 12: note = Xaa is L or E
  VAR_SEQ 16: note = Xaa is A or E
  MOD_RES 17: note = BOUND AT THE C TERM TO CterpolyE
XXXXGGXXXLLXVIVXV

SEQ ID NO: 4 - protein, 18 aa, synthetic construct
  REGION 1..18: note = antagonistic peptide
  VAR_SEQ 1: note = Xaa is I or L
  VAR_SEQ 2: note = Xaa is V or T
  VAR_SEQ 3: note = Xaa is G or S
  VAR_SEQ 4: note = Xaa is I or G or L
  VAR_SEQ 5: note = Xaa is V or G
  VAR_SEQ 8..9: note = Xaa is V or G
  VAR_SEQ 10: note = Xaa is L or E
  VAR_SEQ 10: note = Xaa is L or E
  VAR_SEQ 13: note = Xaa can be any naturally occurring amino acid
  VAR_SEQ 17: note = Xaa is A or E
  MOD_RES 18: note = BOUND AT THE C TERM TO CterpolyE
XXXXXGGXXXLLXVIVXV

SEQ ID NO: 5 - protein, 19 aa, synthetic construct
  REGION 1..19: note = antagonistic peptide
  VAR_SEQ 1: note = Xaa is A or V
  VAR_SEQ 2: note = Xaa is I or L
  VAR_SEQ 3: note = Xaa is V or T
  VAR_SEQ 4: note = Xaa is G or S
  VAR_SEQ 5: note = Xaa is I or G or L
  VAR_SEQ 6: note = Xaa is G or V
  VAR_SEQ 9..10: note = Xaa is G or V
  VAR_SEQ 11: note = Xaa is L or E
  VAR_SEQ 14: note = Xaa is L or E
  VAR_SEQ 18: note = Xaa is A or E
  MOD_RES 19: note = BOUND AT THE C TERM TO CterpolyE
XXXXXXGGXXXLLXVIVXV

SEQ ID NO: 6 - protein, 20 aa, synthetic construct
  REGION 1..20: note = antagonistic peptide
  VAR_SEQ 1: note = Xaa is P or I
  VAR_SEQ 2: note = Xaa is A or V
  VAR_SEQ 3: note = Xaa is I or L
  VAR_SEQ 4: note = Xaa is V or T
  VAR_SEQ 5: note = Xaa is G or S
  VAR_SEQ 6: note = Xaa is I or G or L
  VAR_SEQ 7: note = Xaa is G or V
  VAR_SEQ 10..11: note = Xaa is G or V
  VAR_SEQ 12: note = Xaa is L or E
  VAR_SEQ 15: note = Xaa is L or E
  VAR_SEQ 19: note = Xaa is A or E
  MOD_RES 20: note = BOUND AT THE C TERM TO CterpolyE
XXXXXXXGGXXXLLXVIVXV

SEQ ID NO: 7 - protein, 21 aa, synthetic construct
  REGION 1..21: note = antagonistic peptide
  VAR_SEQ 1: note = Xaa is I or L
  VAR_SEQ 2: note = Xaa is P or I
  VAR_SEQ 3: note = Xaa is A or V
  VAR_SEQ 4: note = Xaa is I or L
  VAR_SEQ 5: note = Xaa is V or T
  VAR_SEQ 6: note = Xaa is G or S
  VAR_SEQ 7: note = Xaa is I or G or L
  VAR_SEQ 8: note = Xaa is G or V
  VAR_SEQ 11..12: note = Xaa is G or V
  VAR_SEQ 13: note = Xaa is L or E
  VAR_SEQ 16: note = Xaa is L or E
  VAR_SEQ 20: note = Xaa is A or E
  MOD_RES 21: note = BOUND AT THE C TERM TO CterpolyE
XXXXXXXXGGXXXLLXVIVXV

SEQ ID NO: 8 - protein, 22 aa, synthetic construct
  REGION 1..22: note = antagonistic peptide
  VAR_SEQ 2: note = Xaa is I or L
  VAR_SEQ 3: note = Xaa is P or I
  VAR_SEQ 4: note = Xaa is A or V
  VAR_SEQ 5: note = Xaa is I or L
  VAR_SEQ 6: note = Xaa is V or T
  VAR_SEQ 7: note = Xaa is G or S
  VAR_SEQ 8: note = Xaa is I or G or L
  VAR_SEQ 9: note = Xaa is G or V
  VAR_SEQ 12..13: note = Xaa is G or V
  VAR_SEQ 14: note = Xaa is L or E
  VAR_SEQ 17: note = Xaa is L or E
  VAR_SEQ 21: note = Xaa is A or E
  MOD_RES 22: note = BOUND AT THE C TERM TO CterpolyE
TXXXXXXXXGGXXXLLXVIVXV

SEQ ID NO: 9 - protein, 28 aa, synthetic construct
  REGION 1..28: note = antagonistic peptide
  VAR_SEQ 1: note = Xaa is G or S
  VAR_SEQ 2: note = Xaa is I or G or L
  VAR_SEQ 3: note = Xaa is G or V
  VAR_SEQ 6..7: note = Xaa is G or V
  VAR_SEQ 8: note = Xaa is L or E
  VAR_SEQ 11: note = Xaa is L or E
  VAR_SEQ 17: note = Xaa is A or LI
  VAR_SEQ 19: note = Xaa is E or D
  VAR_SEQ 20: note = Xaa is D or E
  VAR_SEQ 21: note = Xaa is D, E, or is absent
  VAR_SEQ 22: note = Xaa is D, E, or is absent
  VAR_SEQ 23: note = Xaa is D, E or is absent
  VAR_SEQ 24: note = Xaa is D, E, or is absent
  VAR_SEQ 25: note = Xaa is D, E, or is absent
  VAR_SEQ 26: note = Xaa is D, E, or is absent
  VAR_SEQ 27: note = Xaa is D, E, or is absent
  VAR_SEQ 28: note = Xaa is D, E, or is absent
XXXGGXXXLLXVIVEVXYXXXXXXXXXX

SEQ ID NO: 10 - protein, 26 aa, synthetic construct
  REGION 1..26: note = antagonistic peptide
  VAR_SEQ 4: note = Xaa is G or S
AITXLVGGVGLLLEVIVEVAYEEEEE

SEQ ID NO: 11 - protein, 24 aa, synthetic construct
  REGION 1..24: note = antagonistic peptide
  VAR_SEQ 2: note = Xaa is G or S
TXLVGGVGLLLEVIVEVAYEEEEE

SEQ ID NO: 12 - protein, 23 aa, synthetic construct
  REGION 1..23: note = antagonistic peptide
  VAR_SEQ 1: note = Xaa is G or S
XLVGGVGLLLEVIVEVAYEEEEE

SEQ ID NO: 13 - protein, 30 aa, synthetic construct
  REGION 1..30: note = antagonistic peptide
  VAR_SEQ 7: note = Xaa is G or S
TLPAIVXIGGGGGELLLVIVEVLIYEEEEE

SEQ ID NO: 14 - protein, 29 aa, synthetic construct
  REGION 1..29: note = antagonistic peptide
  VAR_SEQ 7: note = Xaa can be any naturally occurring amino acid
TLPAITXLVGGVGLLLEVIVEVAYEEEEE

SEQ ID NO: 15 - protein, 29 aa, synthetic construct
  REGION 1..29: note = antagonistic peptide
  VAR_SEQ 7: note = Xaa is G or S
TLPAIVXIGGGVVLLLEVIVEVAYEEEEE

SEQ ID NO: 16 - protein, 26 aa, synthetic construct
  REGION 1..26: note = KVASIR
AITGLVGGVGLLLEVIVEVAYEEEEE

SEQ ID NO: 17 - protein, 24 aa, synthetic construct
  REGION 1..24: note = GERD
TGLVGGVGLLLEVIVEVAYEEEEE

SEQ ID NO: 18 - protein, 23 aa, synthetic construct
  REGION 1..23: note = THRUD
GLVGGVGLLLEVIVEVAYEEEEE

SEQ ID NO: 19 - protein, 30 aa, synthetic construct
  REGION 1..30: note = RATI
TLPAIVGIGGGGGELLLVIVEVLIYEEEEE

SEQ ID NO: 20 - protein, 29 aa, synthetic construct
  REGION 1..29: note = GUNGNIR
TLPAITGLVGGVGLLLEVIVEVAYEEEEE

SEQ ID NO: 21 - protein, 29 aa, synthetic construct
  REGION 1..29: note = MIMMING
TLPAIVSIGGGVVLLLEVIVEVAYEEEEE

SEQ ID NO: 22-26 - skipped (5 entries)

SEQ ID NO: 27 - protein, 15 aa, synthetic construct
  REGION 1..15: note = antagonistic peptide
  VAR_SEQ 1: note = Xaa is L or I
  VAR_SEQ 2: note = Xaa is P or I
  VAR_SEQ 3: note = Xaa is A or V
  VAR_SEQ 4: note = Xaa is I or L
  VAR_SEQ 5: note = Xaa is V or T
  VAR_SEQ 6: note = Xaa is G or S
  VAR_SEQ 7: note = Xaa is I or G or L
  VAR_SEQ 8: note = Xaa is G or V
  VAR_SEQ 11..12: note = Xaa is G or V
  VAR_SEQ 13: note = Xaa is L or E
  MOD_RES 1: note = Bound at the N term to NterGp
XXXXXXXXGGXXXLL

SEQ ID NO: 28 - protein, 16 aa, synthetic construct
  REGION 1..16: note = antagonistic peptide
  VAR_SEQ 1: note = Xaa is L or I
  VAR_SEQ 2: note = Xaa is P or I
  VAR_SEQ 3: note = Xaa is A or V
  VAR_SEQ 4: note = Xaa is I or L
  VAR_SEQ 5: note = Xaa is V or T
  VAR_SEQ 6: note = Xaa is G or S
  VAR_SEQ 7: note = Xaa is I or G or L
  VAR_SEQ 8: note = Xaa is G or V
  VAR_SEQ 11..12: note = Xaa is G or V
  VAR_SEQ 13: note = Xaa is L or E
  VAR_SEQ 16: note = Xaa is L or E
  MOD_RES 1: note = Bound at the N term to NterGp
XXXXXXXXGGXXXLLX

SEQ ID NO: 29 - protein, 17 aa, synthetic construct
  REGION 1..17: note = antagonistic peptide
  VAR_SEQ 1: note = Xaa is L or I
  VAR_SEQ 2: note = Xaa is P or I
  VAR_SEQ 3: note = Xaa is A or V
  VAR_SEQ 4: note = Xaa is I or L
  VAR_SEQ 5: note = Xaa is V or T
  VARIANT 6: note = Xaa is G or S
  VAR_SEQ 7: note = Xaa is I or G or L
  VAR_SEQ 8: note = Xaa is G or V
  VAR_SEQ 11..12: note = Xaa is G or V
  VAR_SEQ 13: note = Xaa is L or E
  VAR_SEQ 16: note = Xaa is L or E
  MOD_RES 1: note = Bound at the N term to NterGp
XXXXXXXXGGXXXLLXV

SEQ ID NO: 30 - protein, 18 aa, synthetic construct
  REGION 1..18: note = antagonistic peptide
  VAR_SEQ 1: note = Xaa is L or I
  VAR_SEQ 2: note = Xaa is P or I
  VAR_SEQ 3: note = Xaa is A or V
  VAR_SEQ 4: note = Xaa is I or L
  VARIANT 5: note = Xaa is V or T
  VAR_SEQ 6: note = Xaa is G or S
  VAR_SEQ 7: note = Xaa is I or G or L
  VAR_SEQ 8: note = Xaa is G or V
  VAR_SEQ 11..12: note = Xaa is G or V
  VAR_SEQ 13: note = Xaa is L or E
  VAR_SEQ 16: note = Xaa is L or E
  MOD_RES 1: note = Bound at the N term to NterGp
XXXXXXXXGGXXXLLXVI

SEQ ID NO: 31 - protein, 19 aa, synthetic construct
  REGION 1..19: note = antagonistic peptide
  VAR_SEQ 1: note = Xaa is L or I
  VAR_SEQ 2: note = Xaa is P or I
  VAR_SEQ 3: note = Xaa is A or V
  VAR_SEQ 4: note = Xaa is I or L
  VAR_SEQ 5: note = Xaa is V or T
  VAR_SEQ 6: note = Xaa is G or S
  VAR_SEQ 7: note = Xaa is I or G or L
  VAR_SEQ 8: note = Xaa is G or V
  VAR_SEQ 11..12: note = Xaa is G or V
  VAR_SEQ 13: note = Xaa is L or E
  VAR_SEQ 16: note = Xaa is L or E
  MOD_RES 1: note = Bound at the N term to NterGp
XXXXXXXXGGXXXLLXVIV

SEQ ID NO: 32 - protein, 20 aa, synthetic construct
  REGION 1..20: note = antagonistic peptide
  VAR_SEQ 1: note = Xaa is L or I
  VAR_SEQ 2: note = Xaa is P or I
  VAR_SEQ 3: note = Xaa is A or V
  VAR_SEQ 4: note = Xaa is I or L
  VAR_SEQ 5: note = Xaa is V or T
  VAR_SEQ 6: note = Xaa is G or S
  VAR_SEQ 7: note = Xaa is I or G or L
  VAR_SEQ 8: note = Xaa is G or V
  VAR_SEQ 11..12: note = Xaa is G or V
  VAR_SEQ 13: note = Xaa is L or E
  VAR_SEQ 16: note = Xaa is L or E
  VAR_SEQ 20: note = Xaa is A or E
  MOD_RES 1: note = Bound at the N term to NterGp
XXXXXXXXGGXXXLLXVIVX

SEQ ID NO: 33 - protein, 21 aa, synthetic construct
  REGION 1..21: note = antagonistic peptide
  VAR_SEQ 1: note = Xaa is L or I
  VAR_SEQ 2: note = Xaa is P or I
  VAR_SEQ 3: note = Xaa is A or V
  VAR_SEQ 4: note = Xaa is I or L
  VAR_SEQ 5: note = Xaa is V or T
  VAR_SEQ 6: note = Xaa is G or S
  VAR_SEQ 7: note = Xaa is I or G or L
  VAR_SEQ 8: note = Xaa is G or V
  VAR_SEQ 11..12: note = Xaa is G or V
  VAR_SEQ 13: note = Xaa is L or E
  VAR_SEQ 16: note = Xaa is L or E
  VAR_SEQ 20: note = Xaa is A or E
  MOD_RES 1: note = Bound at the N term to NterGp
XXXXXXXXGGXXXLLXVIVXV

SEQ ID NO: 34 - protein, 22 aa, synthetic construct
  REGION 1..22: note = antagonistic peptide
  VAR_SEQ 1: note = Xaa is L or I
  VAR_SEQ 2: note = Xaa is P or I
  VAR_SEQ 3: note = Xaa is A or V
  VAR_SEQ 4: note = Xaa is I or L
  VAR_SEQ 5: note = Xaa is V or T
  VARIANT 6: note = Xaa is G or S
  VAR_SEQ 7: note = Xaa is I or G or L
  VAR_SEQ 8: note = Xaa is G or V
  VAR_SEQ 11..12: note = Xaa is G or V
  VAR_SEQ 13: note = Xaa is L or E
  VAR_SEQ 16: note = Xaa is L or E
  VAR_SEQ 20: note = Xaa is A or E
  MOD_RES 1: note = Bound at the N term to NterGp
XXXXXXXXGGXXXLLXVIVXVL

SEQ ID NO: 35 - protein, 23 aa, synthetic construct
  REGION 1..23: note = antagonistic peptide
  VAR_SEQ 1: note = Xaa is L or I
  VAR_SEQ 2: note = Xaa is P or I
  VAR_SEQ 3: note = Xaa is A or V
  VAR_SEQ 4: note = Xaa is I or L
  VAR_SEQ 5: note = Xaa is V or T
  VAR_SEQ 6: note = Xaa is G or S
  VAR_SEQ 7: note = Xaa is I or G or L
  VAR_SEQ 8: note = Xaa is G or V
  VAR_SEQ 11..12: note = Xaa is G or V
  VAR_SEQ 13: note = Xaa is L or E
  VAR_SEQ 16: note = Xaa is L or E
  VAR_SEQ 20: note = Xaa is A or E
  MOD_RES 1: note = Bound at the N term to NterGp
XXXXXXXXGGXXXLLXVIVXVLI

SEQ ID NO: 36 - protein, 21 aa, synthetic construct
  REGION 1..21: note = antagonistic peptide
  VAR_SEQ 1: note = Xaa is A or V
  VAR_SEQ 2: note = Xaa is I or L
  VAR_SEQ 3: note = Xaa is V or T
  VAR_SEQ 4: note = Xaa is G or S
  VAR_SEQ 5: note = Xaa is I or G or L
  VAR_SEQ 6: note = Xaa is G or V
  VAR_SEQ 9..10: note = Xaa is G or V
  VAR_SEQ 11: note = Xaa is L or E
  VAR_SEQ 14: note = Xaa is L or E
  VAR_SEQ 18: note = Xaa is A or E
  MOD_RES 21: note = Bound at the C term to CterGp
XXXXXXGGXXXLLXVIVXVLI

SEQ ID NO: 37 - protein, 20 aa, synthetic construct
  REGION 1..20: note = antagonistic peptide
  VAR_SEQ 1: note = Xaa is I or L
  VAR_SEQ 2: note = Xaa is V or T
  VAR_SEQ 3: note = Xaa is G or S
  VAR_SEQ 4: note = Xaa is I or G or L
  VAR_SEQ 5: note = Xaa is G or V
  VAR_SEQ 8..9: note = Xaa is G or V
  VAR_SEQ 10: note = Xaa is L or E
  VAR_SEQ 13: note = Xaa is L or E
  VAR_SEQ 17: note = Xaa is A or E
  MOD_RES 20: note = Bound at the C term to CterGp
XXXXXGGXXXLLXVIVXVLI

SEQ ID NO: 38 - protein, 19 aa, synthetic construct
  REGION 1..19: note = antagonistic peptide
  VAR_SEQ 1: note = Xaa is V or T
  VAR_SEQ 2: note = Xaa is G or S
  VAR_SEQ 3: note = Xaa is I or G or L
  VAR_SEQ 4: note = Xaa is G or V
  VAR_SEQ 7..8: note = Xaa is G or V
  VAR_SEQ 9: note = Xaa is L or E
  VAR_SEQ 12: note = Xaa is L or E
  VAR_SEQ 16: note = Xaa is A or E
  MOD_RES 19: note = Bound at the C term to CterGp
XXXXGGXXXLLXVIVXVLI

SEQ ID NO: 39 - protein, 18 aa, synthetic construct
  REGION 1..18: note = antagonistic peptide
  VAR_SEQ 1: note = Xaa is G or S
  VAR_SEQ 2: note = Xaa is I or G or L
  VAR_SEQ 3: note = Xaa is G or V
  VAR_SEQ 6..7: note = Xaa is G or V
  VAR_SEQ 8: note = Xaa is L or E
  VAR_SEQ 11: note = Xaa is L or E
  VAR_SEQ 15: note = Xaa is A or E
XXXGGXXXLLXVIVXVLI

SEQ ID NO: 40 - protein, 18 aa, synthetic construct
  REGION 1..18: note = antagonistic peptide
  VAR_SEQ 10: note = Xaa is G or S
KGDWLPAITXLVGGVGLL

SEQ ID NO: 41 - protein, 18 aa, synthetic construct
  REGION 1..18: note = antagonistic peptide
  VAR_SEQ 10: note = Xaa is G or S
KGDWLPAIVXIGGGVVLL

SEQ ID NO: 42 - protein, 18 aa, synthetic construct
  REGION 1..18: note = antagonistic peptide
  VAR_SEQ 10: note = Xaa is G or S
KGDWIPALVXGGGGGGLL

SEQ ID NO: 43 - protein, 18 aa, synthetic construct
  REGION 1..18: note = antagonistic peptide
  VAR_SEQ 10: note = Xaa is G or S
KGDWLPALVXIGGGVGLL

SEQ ID NO: 44 - protein, 18 aa, synthetic construct
  REGION 1..18: note = antagonistic peptide
  VAR_SEQ 10: note = Xaa is G or S
KGDWIPALVXLGGGGGLL

SEQ ID NO: 45 - protein, 14 aa, synthetic construct
  REGION 1..14: note = antagonistic peptide
  VAR_SEQ 10: note = Xaa is G or S
KGDWLIAIVXIGGG

SEQ ID NO: 46 - protein, 14 aa, synthetic construct
  REGION 1..14: note = antagonistic peptide
  VAR_SEQ 10: note = Xaa is G or S
KGDWLPVIVXIGGG

SEQ ID NO: 47 - protein, 18 aa, synthetic construct
  REGION 1..18: note = antagonistic peptide
  VAR_SEQ 10: note = Xaa is G or S
KGDWLPAIVXIGGGGGLL

SEQ ID NO: 48 - protein, 17 aa, synthetic construct
  REGION 1..17: note = antagonistic peptide
  VAR_SEQ 10: note = Xaa is G or S
KGDWLPAIVXIGGGGGL

SEQ ID NO: 49 - protein, 16 aa, synthetic construct
  REGION 1..16: note = antagonistic peptide
  VAR_SEQ 10: note = Xaa is G or S
KGDWLPAIVXIGGGGG

SEQ ID NO: 50 - protein, 15 aa, synthetic construct
  REGION 1..15: note = antagonistic peptide
  VAR_SEQ 10: note = Xaa is G or S
KGDWLPAIVXIGGGG

SEQ ID NO: 51 - protein, 14 aa, synthetic construct
  REGION 1..14: note = antagonistic peptide
  VAR_SEQ 10: note = Xaa is G or S
KGDWLPAIVXIGGG

SEQ ID NO: 52 - protein, 18 aa, synthetic construct
  REGION 1..18: note = BALDR
KGDWLPAITGLVGGVGLL

SEQ ID NO: 53 - protein, 18 aa, synthetic construct
  REGION 1..18: note = FREYR
KGDWLPAIVSIGGGVVLL

SEQ ID NO: 54 - protein, 18 aa, synthetic construct
  REGION 1..18: note = BRAGI
KGDWIPALVGGGGGGGLL

SEQ ID NO: 55 - protein, 18 aa, synthetic construct
  REGION 1..18: note = NJORD
KGDWLPALVSIGGGVGLL

SEQ ID NO: 56 - protein, 18 aa, synthetic construct
  REGION 1..18: note = ULLR
KGDWIPALVGLGGGGGLL

SEQ ID NO: 57 - protein, 14 aa, synthetic construct
  REGION 1..14: note = SKOLL
KGDWLIAIVGIGGG

SEQ ID NO: 58 - protein, 14 aa, synthetic construct
  REGION 1..14: note = HATI
KGDWLPVIVGIGGG

SEQ ID NO: 59 - protein, 18 aa, synthetic construct
  REGION 1..18: note = plexA1-S2
KGDWLPAIVGIGGGGGLL

SEQ ID NO: 60 - protein, 17 aa, synthetic construct
  REGION 1..17: note = FJALAR
KGDWLPAIVGIGGGGGL

SEQ ID NO: 61 - protein, 16 aa, synthetic construct
  REGION 1..16: note = GALAR
KGDWLPAIVGIGGGGG

SEQ ID NO: 62 - protein, 15 aa, synthetic construct
  REGION 1..15: note = IVALDI
KGDWLPAIVGIGGGG

SEQ ID NO: 63 - protein, 14 aa, synthetic construct
  REGION 1..14: note = ALVISS
KGDWLPAIVGIGGG

SEQ ID NO: 64 - protein, 29 aa, synthetic construct
  REGION 1..29: note = SURTR
TLPVIGVIVLVGSAVLELIAEGVYEEEEE

SEQ ID NO: 65 - protein, 29 aa, synthetic construct
  REGION 1..29: note = HODR
TLPAIGVITLVGLGVLELVAEGVYEEEEE

SEQ ID NO: 66 - protein, 18 aa, synthetic construct
  REGION 1..18: note = GULLVEIG
KGDWLPAIVSIGGAVVLL

SEQ ID NO: 67 - protein, 18 aa, synthetic construct
  REGION 1..18: note = SKIRNIR
KGDWLPAIVGIGGSVVLL

SEQ ID NO: 68 - protein, 13 aa, synthetic construct
  REGION 1..13: note = RATATOSK
KGDWLPAIVGIGG

SEQ ID NO: 69 - protein, 19 aa, synthetic construct
  REGION 1..19: note = plexA1-S1
KGDWTLPAIVGIGGGGGLL

SEQ ID NO: 70 - protein, 17 aa, synthetic construct
  REGION 1..17: note = BROKK
KGDWPAIVGIGGGGGLL

SEQ ID NO: 71 - protein, 15 aa, synthetic construct
  REGION 1..15: note = EITRI
KGDWIVGIGGGGGLL

SEQ ID NO: 72 - protein, 31 aa, synthetic construct
  REGION 1..31: note = DRAUPNIR
TLPAIVGIGGGGGLLLEVIVEVLIAYEEEEE

SEQ ID NO: 73 - protein, 29 aa, synthetic construct
  REGION 1..29: note = MJOLLNIR
TLPAITGLVGGVGLLVEVAVEIAYEEEEE

SEQ ID NO: 74 - protein, 13 aa, synthetic construct
  REGION 1..13: note = PLP139-151 fragment
HSLGKWLGHPDKF

SEQ ID NO: 75 - protein, 21 aa, synthetic construct
  REGION 1..21: note = MOG35-55 fragment
MEVGWYRSPFSRVVHLYRNGK

SEQ ID NO: 76 - protein, 5 aa, synthetic construct
  REGION 1..5: note = CterGp
AYKRK

SEQ ID NO: 77 - protein, 5 aa, synthetic construct
  REGION 1..5: note = CterGp
AYKKR

SEQ ID NO: 78 - protein, 5 aa, synthetic construct
  REGION 1..5: note = CterGp
AYKRR

SEQ ID NO: 79 - protein, 5 aa, synthetic construct
  REGION 1..5: note = CterGp
AYRRK

SEQ ID NO: 80 - protein, 5 aa, synthetic construct
  REGION 1..5: note = CterGp
AYRKK

SEQ ID NO: 81 - protein, 29 aa, synthetic construct
  REGION 1..29: note = ODIN
KRKYAILVAVIVLLLLGVGGGIGVIAPLT

SEQ ID NO: 82 - protein, 5 aa, synthetic construct
  REGION 1..5: note = Part of second domain
LPAIT

SEQ ID NO: 83 - protein, 5 aa, synthetic construct
  REGION 1..5: note = Part of second domain
LPAIV

SEQ ID NO: 84 - protein, 5 aa, synthetic construct
  REGION 1..5: note = part of second domain
IPALV

SEQ ID NO: 85 - protein, 5 aa, synthetic construct
  REGION 1..5: note = part of second domain
LPALV

SEQ ID NO: 86 - protein, 5 aa, synthetic construct
  REGION 1..5: note = part of second domain
LIAIV

SEQ ID NO: 87 - protein, 5 aa, synthetic construct
  REGION 1..5: note = part of second domain
LPVIV

SEQ ID NO: 88 - protein, 13 aa, synthetic construct
  REGION 1..13: note = part of second domain
GGLLLLVIVAVLI

SEQ ID NO: 89 - protein, 7 aa, synthetic construct
  REGION 1..7: note = CterpolyE
AYEEEEE

SEQ ID NO: 90 - protein, 8 aa, synthetic construct
  REGION 1..8: note = CterpolyE
LIYEEEEE

SEQ ID NO: 91 - protein, 11 aa, synthetic construct
  REGION 1..11: note = part of second domain
VGLLLEVIVEV

SEQ ID NO: 92 - protein, 10 aa, synthetic construct
  REGION 1..10: note = part of second domain
GGELLLVIVE

SEQ ID NO: 93 - protein, 11 aa, synthetic construct
  REGION 1..11: note = part of second domain
VVLLLEVIVEV

SEQ ID NO: 94 - protein, 30 aa, synthetic construct
TLPAITGVVVGGVGLLLEVIVEVAYEEEEE

SEQ ID NO: 95 - protein, 29 aa, synthetic construct
TLPAITGLVVGVGLLLEVIVEVAYEEEEE

SEQ ID NO: 96 - protein, 28 aa, synthetic construct
TLPAITGLVGGVGLLLEVIVEVAYEEEE

SEQ ID NO: 97 - protein, 27 aa, synthetic construct
TLPAITGLVGGVGLLLEVIVEVAYEEE

SEQ ID NO: 98 - protein, 26 aa, synthetic construct
TLPAITGLVGGVGLLLEVIVEVAYEE

SEQ ID NO: 99 - protein, 29 aa, synthetic construct
TLPAITGLVGGVGLLLEVIVEVAYDDDDD

SEQ ID NO: 100 - protein, 29 aa, synthetic construct
TLPAITGLVGGVVLLLEVIVEVAYEEEEE

SEQ ID NO: 101 - protein, 29 aa, synthetic construct
TLPAITGLVGGVGLVLEVIVEVAYEEEEE

SEQ ID NO: 102 - protein, 29 aa, synthetic construct
TLPAITGLVGGVGLLLVVIVEVAYEEEEE

SEQ ID NO: 103 - protein, 29 aa, synthetic construct
TLPAITGLVGGVGLLLEVVVEVAYEEEEE

SEQ ID NO: 104 - protein, 29 aa, synthetic construct
TLPAITGLVGGVGLLLEVIVVVAYEEEEE

SEQ ID NO: 105 - protein, 29 aa, synthetic construct
  NON_STD 1..29: note = D amino acids
TLPAITGLVGGVGLLLEVIVEVVYEEEEE

SEQ ID NO: 106 - protein, 29 aa, synthetic construct
EEEEEYAVEVIVELLLGVGGVLGTIAPLT

SEQ ID NO: 107 - protein, 26 aa, synthetic construct
TLPAITGLVGGVGLLLEVIVEVAYDD

SEQ ID NO: 108 - protein, 29 aa, synthetic construct
TLPAITGLVGGVGLLLEVIVEVAYDEDED

SEQ ID NO: 109 - protein, 23 aa, synthetic construct
TGLVGGVGLLLEVIVEVAYEEEE

SEQ ID NO: 110 - protein, 22 aa, synthetic construct
TGLVGGVGLLLEVIVEVAYEEE

SEQ ID NO: 111 - protein, 29 aa, synthetic construct
  MOD_RES 1: note = D-Threonine
TLPAITGLVGGVGLLLEVIVEVAYEEEEE

SEQ ID NO: 112 - protein, 29 aa, synthetic construct
  MOD_RES 21: note = D-glutamic acid
TLPAITGLVGGVGLLLEVIVEVAYEEEEE

SEQ ID NO: 113 - protein, 29 aa, synthetic construct
TLPAITGLVGGVGLLVEVIVEVAYEEEEE

SEQ ID NO: 114 - protein, 29 aa, synthetic construct
TLPAITGLVGGVGLLLEVAVEVAYEEEEE

SEQ ID NO: 115 - protein, 29 aa, synthetic construct
TLPAITGLVGGVGLLLEVIVEIAYEEEEE

SEQ ID NO: 116 - protein, 4 aa, synthetic construct
KGDW

SEQ ID NO: 117 - protein, 11 aa, synthetic construct
VGLLVEVIVEV

SEQ ID NO: 118 - protein, 11 aa, synthetic construct
VGLVLEVIVEV

SEQ ID NO: 119 - protein, 28 aa, synthetic construct
  VAR_SEQ 1: note = X is G or S
  VAR_SEQ 6..7: note = X is G or V
  VAR_SEQ 8: note = X is L or E
  VAR_SEQ 10: note = X is L or V
  VAR_SEQ 11: note = X is L or E
  VAR_SEQ 19..20: note = X is D or E
  VAR_SEQ 21..28: note = X is D, E or is absent
XLVGGXXXLXXVIVEVAYXXXXXXXXXX